(19) United States
(12) Patent Application Publication
(10) Pub. No.: US 2004/0180358 A1
(43) Pub. Date: Sep. 16, 2004

(54) Methods for predicting therapeutic response to agents acting on the growth hormone receptor

(76) Inventor: Pierre Bougneres, Chaville (FR)

Correspondence Address: DINSMORE & SHOHL, LLP, 1900 CHEMED CENTER, 255 EAST FIFTH STREET, CINCINNATI, OH, 45202, US

(21) Appl. No.: 10/703,033

(22) Filed: Nov. 6, 2003

Related U.S. Application Data
(60) Provisional application No. 60/434,861, filed on Dec. 19, 2002.

(51) Int. Cl. C12Q 1/68

(52) U.S. Cl. 435/6

(57) ABSTRACT

Methods of producing a subject's response to an agent capable of binding to a growth hormone receptor (GHR) protein comprise determining in the subject the preence or absence of an allele of the GHR gene, wherein the allele is coordinated with the likelihood of having an increased or decreased positive response to the agent, thereby identifying the subject as having an increased or decreased likelihood of responding to treatment with the agent.

### FIELD OF THE INVENTION

This invention relates to methods for predicting the magnitude of a subject's therapeutic response to agents that act on the growth hormone receptor. Preferred aspects include methods for increasing the height of human subjects having short stature, and for treating obesity and acromegaly.

### BACKGROUND

Most children with significant short stature do not have growth hormone deficiency (GHD) as classically defined by the GH response to provocative stimuli. Once known causes of short stature have been excluded, these subjects are classified with various terms, including familial short stature, constitutional delay of growth, “idiopathic” short stature (ISS). The case of children born short to parents of normal size are called ‘intra uterine growth retardation’ (IUGR). Children born short for their term are called ‘small for gestational age’ (SGA). Some, and presumably a large number of, of these children may not reach their genetic potential for height, although results from large-scale longitudinal studies have not been reported. Since there are so many factors that contribute to normal growth and development, it is likely that subjects with ISS, IUGR, SGA as defined, are heterogeneous with regard to their etiology of short stature. Despite not being classically GH deficient, most children with ISS respond to treatment with GH, although not all equally well.

Many investigators have searched for disturbances in spontaneous GH secretion in this set of subjects. One hypothesis suggests that some of these subjects have inadequate secretion of endogenous GH under physiologic conditions, but are able to demonstrate a rise in GH in response to pharmacologic stimuli, as in traditional GH stimulation tests. This disorder has been termed “GH neurosecretory dysfunction,” and the diagnosis rests on the demonstration of an abnormal circulating GH pattern on prolonged serum sampling. Numerous investigators have reported results of such studies, and have found this abnormality to be only occasionally present. Other investigators have postulated that these subjects have “bioinactive GH;” however, this has not yet been demonstrated conclusively.

When the GH receptor (GHR) was cloned, it was shown that the major GH binding activity in blood was due to a protein which derives from the same gene as the GHR and corresponds to the extracellular domain of the full-length GHR. Almost all subjects with growth hormone insensitivity (or Laron) syndrome (GHIS) lack growth hormone receptor binding activity and have absent or very low GH-binding protein (GHBP) activity in blood. Such subjects have a mean height standard deviation score (SDS) of about −5 to −6, are resistant to GH treatment, and have increased serum concentrations of GH and low serum concentrations of insulin-like growth factor (IGF-I). They respond to treatment with IGF-I. In subjects with defects in the extracellular domain of the GHR, the lack of functional GHBP in the circulation can serve as a marker for the GH insensitivity.

Subjects with ISS who are treated with exogenous GH have shown differing rates of response to treatment. In particular, many children respond somewhat, but not completely, to GH treatment. These subjects have an increase of their growth rates that is only about half that of children that respond fully. The childrens' total height gain following the course of treatment is therefore reduced versus that of children that respond fully, depending on treatment duration. One way of improving the treatment of subjects that do not respond fully has been to increase the GH dosage, which has resulted in somewhat improved growth rates and total height gain. However, increased GH dosage is not desirable for all subjects due to potential side effects. Increased GH dosage also entails increased cost. Unfortunately there is at present no method to identify subjects likely to be less responsive prior to a lengthy treatment and observation period.

There is therefore a need in the art for methods that can be used to identify a subset of subjects who exhibit diminished response rates to treatment with GH. There is also a need for methods that allow the development of improved medicaments for the treatment of subjects who have diminished response to exogenous GH. There is also a need in the art for methods that can be used to identify a subset of subjects who exhibit increased response rates to GH and a need for methods that allow the development of improved medicaments for the treatment of subjects who have increased response to GH.

The subjects may include for example individuals of short stature including, or individuals suffering from obesity, infection, or diabetes; acromegaly or gigantism conditions which could be associated with lactogenic, diabetogenic, lipolytic and protein anabolic effects; conditions associated with sodium and water retention; metabolic syndromes; mood and sleep disorders, cancer, cardiac disease and hypertension.

### SUMMARY OF THE INVENTION

The present invention is based on the discovery that human subjects carrying a growth hormone receptor (GHR) allele having an exon 3 deletion (GHRd3) have a greater positive response to treatment with an agent acting via the GHR pathway than subjects not carrying the GHRd3 allele. In particular, subjects carrying the GHRd3 allele demonstrated a greater positive response to treatment with recombinant growth hormone (GH) than subjects not carrying said GHRd3 allele. Over the course of treatment with recombinant GH, subjects having ISS, IUGR or SGA and carrying the GHRd3 had a gain in growth rates approximately double that of ISS subjects that were homozygous for the GHRfl allele. Their total height change is increased in proportion off this effect. GH activity is mediated by the GH receptor (GHR), discussed above. It has been shown that two molecules of GHR interact with a single molecule of GH (Cunningham et al., (1991) Science 254: 821-825; de Vos et al., (1992) Science 255: 306-312; Sundstrom et al., (1996) J. Biol. Chem. 271: 32197-32203; and Clackson et al., (1998) J. Mol. Biol. 277: 1111-1128. The binding happens at two unique GHR binding sites on GH and a common binding pocket on the extracellular domain of two receptors. Site 1 on the GH molecule has a higher affinity than Site 2, and receptor dimerization is thought to occur sequentially, with one receptor binding to site 1 on GH followed by recruitment of a second receptor to site 2. Cunningham et al (1991, supra) have proposed that receptor dimerization is the key event leading to signal activation and that dimerization is driven by GH binding (Ross et al, J. Clin. Endocrinol. & Metabolism (2001) 86(4): 1716-171723. Upon ligand binding, GHRs are internalized rapidly (Maamra et al, (1999) J. Biol. Chem 274: 14791-14798; and Harding et al., (1996) J. Biol. Chem. 271: 6708-6712), with a proportion recycled to the cell surface (Roupas et al., (1987) Endocrinol. 121: 1521-1530).

More recently a GHR isoform referred to as GHRd3 was discovered that contains a deletion of exon 3. (Urbanek M et al., Mol Endocrinol 1992 Feb;6(2):279-87; Godowski et al (1989) PNAS USA 86: 8083-8087). The deletion was thought to be the result of an alternative splicing event leading to either the retention of the exclusion of exon 3, corresponding either to the full length GHRfl isoform or the exon 3-deleted GHRd3 isoform. Several contradictory results followed the identification of the GHRd3 isoform. Reports proposed that the GHRd3 isoform was subject to tissue-specific splicing, that the expression pattern was developmentally regulated, while other reports proposed that the GHRd3 isoform was specific to an individual. Another report suggested that splicing resulted from a genetic polymorphism that is transmitted as a Mendelian trait and alters splicing (Stallings-Mann et al., (1996) P.N.A.S U.S.A. 94: 12394-12399). Finally, Pantel et al. ((2000), J. Biol. Chem. 275 (25): 18664-18669), demonstrated upon analysis of the GHR locus that in humans the GHRd3 isoform is transcribed from a GHR allele that carried a 2.7 kb genomic deletion spanning exon 3. Pantel further identified two flanking retroelements in the genomic DNA samples from individuals who express only GHRfl, but only a single a retroelement in the DNA of individuals expression GHRd3, suggesting that the exon 3 deletion is the result of a homologous recombination event between the two retroelements located on the same GHRfl allele

The hGHRd3 protein differs from the full length hGHR (GHRfl) by a deletion of 22 amino acids within the extracellular domain of the receptor. The GHRd3 isoform encodes a stable and functional GHR protein (Urbanek et al., (1993) J. Biol. Chem. 268 (25): 19025-19032). While Urbanek et al. (1993) reported that the GHRd3 isoform is stably integrated into the cell membrane and binds and internalizes ligand as efficiently as hGHR, no functional differences from the GHRfl isoform were identified

The present invention relates to the identification of a GHR allele and isoform as an important factor contributing to differences in positive response to exogenous GH. The invention thus provides a method to predict the degree of a positive response to treatment with compounds that act via the GHR pathway, or preferably compounds that bind the GHR, such as GH compositions. The methods allow the classification of patients a priori as e.g. either high or low responders. Allowing a treatment to be adapted for a particular subject results in economic benefits and/or reduced side effects (e.g. from use of the appropriate dosage of GH compositions or from the use of a compound to which subjects to not show diminished GHR response).

The invention also demonstrates that subjects heterozygous for the GHRd3 and GHRfl allele show growth rates and height changes in response to treatment with GH that are greater than subjects homozygous for the GHRfl allele. The invention thus provides methods of detecting and diagnosing diminished GHR response or GHR activity in an individual who is homozygous for the GHRfl allele. Diminished GHR activity can be the result for example of diminished GHR levels, expression or protein activity. Also provided are methods of detecting and diagnosing increased GHR response or GHR activity in an individual who is homozygous or heterozygous for the GHRd3 allele. Detecting increased or diminished GHR activity is predicted to be useful in the treatment of a variety of disorders treatable using therapeutic agents that act via the GHR pathway. Examples include treatment of short stature (e.g. preferably ISS, IUGR, or SGA), obesity, infection, or diabetes; acromegaly or gigantism conditions which could be associated with lactogenic, diabetogenic, lipolytic and protein anabolic effects; conditions associated with sodium and water retention; metabolic syndromes; mood and sleep disorders, cancer, cardiac disease and hypertension. Preferred examples include agents that bind the GHR protein such as recombinant GH compositions acting as GHR agonists or antagonists.

The present invention thus provides methods for determining or predicting GHR-mediated activity, including methods of predicting GHR response to treatment, and methods of identifying a subject at risk for or diagnosing a condition related to diminished GHR activity. Preferably the invention provides methods of predicting a subject's response to an agent capable of interacting with (e.g. binding to) a GHR polypeptide.

Accordingly, in one aspect, the invention discloses a method of predicting a subject's response to an agent capable of binding a GHR protein, comprising determining in the subject the presence or absence of an allele of the GHR gene, wherein the allele is correlated with a likelihood of having an increased or decreased positive response to said agent, thereby identifying the subject as having an increased or decreased likelihood of responding to treatment with said agent.

The invention also provides a method of predicting a subject's response to an agent for the treatment of a condition selected from the group consisting of short stature (e.g. preferably ISS, IUGR, or SGA), obesity, infection, or diabetes; acromegaly or gigantism conditions which could be associated with lactogenic, diabetogenic, lipolytic and protein anabolic effects; conditions associated with sodium and water retention; metabolic syndromes; mood and sleep disorders, cancer, cardiac disease and hypertension, said method comprising: determining in the subject the presence or absence of an allele of the GHR gene, wherein the allele is correlated with a likelihood of having an increased or decreased positive response to said agent, thereby identifying the subject as having an increased or decreased likelihood of responding to treatment with said agent. In preferred aspects, the invention provides a method of predicting a subject's response to an agent for increasing the height of a subject, comprising determining in the subject the presence or absence of an allele of the GHR gene, wherein the allele is correlated with a likelihood of having an increased or decreased positive response to said agent, thereby identifying the subject as having an increased or decreased likelihood of responding to treatment with said agent.

Preferably, the methods of the invention comprise determining in the subject the presence or absence of a GHR allele having a deletion, insertion or subsitution of one or more nucleic acids in exon 3, or most preferably having a deletion of substantially the entire exon 3.

Also provided is a method of identifying a subject having an increased or decreased likelihood of treating a disorder or condition with an agent capable of binding to a GHR protein, comprising:

a) correlating the presence of an allele of the GHR gene with a subject's response to an agent capable of binding to a GHR protein; and

b) detecting the allele of step a) in the subject, thereby identifying a subject an increased or decreased likelihood of responding to treatment with said agent.

In yet another aspect, encompassed is a method of identifying an allele in the GHR gene correlated with an increased or decreased likelihood of treating a disorder or condition with an agent capable of binding to a GHR protein, comprising:

a) determining in a subject the presence of an allele of the GHR gene; and

b) correlating the presence of the allele of step (a) with an increased or decreased likelihood of treating a disorder or condition with an agent capable of binding to a GHR protein, thereby identifying an allele correlated with an increased or decreased likelihood of responding to treatment with said agent.

Said disorder or condition may be a condition selected from the group consiting of: short stature (e.g. preferably ISS, IUGR, or SGA), obesity, infection, or diabetes; acromegaly or gigantism conditions which could be associated with lactogenic, diabetogenic, lipolytic and protein anabolic effects; conditions associated with sodium and water retention; metabolic syndromes; mood and sleep disorders, cancer, cardiac disease and hypertension.

Most preferably, the methods of the invention comprise determining the genotype of a subject at exon 3 of the GHR gene, wherein the presence of exon 3 is indicative of said subject suffering from or having an increased risk for a condition related to diminished GHR response, or wherein a deletion in exon 3 is indicative of said subject having an decreased risk for a condition related to diminished GHR response.

The methods of the invention can be used particularly advantageously in methods of treatment. Preferably, said genotype is indicative of the efficacy or therapeutic benefits of said therapy. In one example, the methods of the invention are used to determine the amount of a medicament to be administered to a subject. In another example, the methods are used to assess the therapeutic response of subjects in a clinical trial or to select subjects for inclusion in a clinical trial. For instance, the methods of the invention may comprise determining the genotype of a subject at exon 3 of the GHR gene, wherein said genotype places said subject into a subgroup in a clinical trial or in a subgroup for inclusion in a clinical trial.

The invention also provides a method for treating a subject, the method comprising:

(a) determining in the subject the presence or absence of an allele of the GHR gene, wherein the allele is correlated with a likelihood of having an increased or decreased positive response to an agent capable of binding to a GHR protein or acting via the GHR pathway; and

(b) selecting or determining an effective amount of said agent to administer to said subject.

An agent capable of binding to a GHR protein or acting via the GHR pathway according to any of the methods of the invention is preferably an agent effective in the treatment of a condition selected from the group consiting of: short stature (e.g. preferably ISS, IUGR, or SGA), obesity, infection, or diabetes; acromegaly or gigantism conditions which could be associated with lactogenic, diabetogenic, lipolytic and protein anabolic effects; conditions associated with sodium and water retention; metabolic syndromes; mood and sleep disorders, cancer, cardiac disease and hypertension.

In particular, the invention provides methods for treating a subject comprising:

(a) determining in the subject the presence or absence of an allele of the GHR gene, wherein the allele is correlated with a likelihood of having an increased or decreased positive response to an agent capable of treating a condition selected from the group consisting of short stature (e.g. preferably ISS, IUGR, or SGA), obesity, infection, or diabetes; acromegaly or gigantism conditions which could be associated with lactogenic, diabetogenic, lipolytic and protein anabolic effects; conditions associated with sodium and water retention; metabolic syndromes; mood and sleep disorders, cancer, cardiac disease and hypertension; and

(b) selecting or determining an effective amount of said agent to administer to said subject.

In particularly preferred embodiments, the invention discloses a method for increasing the growth of a subject, the method comprising:

(a) determining in the subject the presence or absence of an allele of the GHR gene, wherein the allele is correlated with a likelihood of having an increased or decreased positive response to an agent capable of increasing the growth of a subject; and

(b) selecting or determining an effective amount of said agent to administer to said subject.

In a preferred aspect, the invention discloses a method for increasing the growth rate of a human subject, said method comprising:

(a) detecting whether the subject has a height less than about 1 standard deviation, or more preferably less than about 2 standard deviations below normal for age and sex,

(b) detecting whether the DNA of the subject encodes a GHRd3 polypeptide; and,

(c) administering to the subject an effective amount of GH that increases the growth rate of the subject. Preferably, the subject will not be a subject having Laron's sydrome.

Preferably, said methods of treating a human subject comprise administering to a subject homozygous for the GHRfl allele an effective dose of an agent which is greater than the effective dose that would be administered to an otherwise identical subject whose DNA encodes a GHRd3 protein.

In preferred aspects, said agent is a GH molecule. Preferably, the effective amount of GH administered to a subject is between about 0.001 mg/kg/day and about 0.2 mg/kg/day; more preferably, the effective amount of GH is between about 0.01 mg/kg/day and about 0.1 mg/kg/day. In other aspects, the effective amount of GH administered to a subject is at least about 0.2 mg/kg/week. In another aspect, the effective amount of GH is at least about 0.25 mg/kg/week. In another aspect, the effective amount of GH is at least about 0.3 mg/kg/week. Preferably, the GH is administered once per day. Preferably the GH is administered by subcutaneous injections. Most preferably, the growth hormone is formulated at a pH of about 7.4 to 7.8.

Another aspect of the invention concerns a method of using a medicament comprising: obtaining a DNA sample from a subject, determining whether the DNA sample contains an allele of the GHR gene associated with an increased positive response to the medicament and/or whether the DNA sample contains an allele of the GHR gene associated with a diminished positive response to the medicament, and administering an effective amount of the medicament to the subject if the DNA sample contains an allele of the GHR gene associated with a increased positive response to the medicament and/or if the DNA sample lacks an allele of the GHR gene associated with a diminished positive response to the medicament.

In another aspect, the invention comprises treating a subject suffering from diminished response to exogenous GH. In this aspect, the invention provides a method of using a medicament comprising: obtaining a DNA sample from a subject, determining whether the DNA sample contains an allele of the GHR gene associated with an increased positive response to the medicament and/or whether the DNA sample contains an allele of the GHR gene associated with a diminished positive response to the medicament, and administering an effective amount of the medicament to the subject if the DNA sample contains an allele of the GHR gene associated with a diminished positive response to the medicament and/or if the DNA sample lacks an allele of the GHR gene associated with an increased positive response to the medicament.

As discussed, the methods comprise determining in the subject the presence or absence of a GHR allele having a deletion, insertion or subsitution of one or more nucleic acids in exon 3, or most preferably having a deletion of substantially the entire exon 3. An allele of the GHR gene associated with an increased positive response to the medicament is a GHR allele lacking exon 3, preferably a GHRd3 allele. An allele of the GHR gene associated with a diminished positive response to the medicament is preferably a GHR allele (GHRfl) containing exon 3 (when a subject is a homozygote for this allele).

The invention also concerns a method for the clinical testing of a medicament, the method comprising the following steps.

administering a medicament to a population of individuals; and

from said population, identifying a first subpopulation of individuals whose DNA encodes a GHRd3 polypeptide isoform and a second subpopulation of individuals whose DNA does not encode a GHRd3 polypeptideisoform.

Said method may further comprise: (a) assessing the response to said medicament in said first subpopulation of individuals; and/or (b) assessing the response to said medicament in said second subpopulation of individuals. Preferably, the response to said medicament is assessed both in said first and said second subpopulation of individuals. Preferably said response is assessed separately in said first and second subpopulation of individuals. Assessing the response to said medicament preferably comprises determining the change in height of a subject.

The invention also concerns a method for the clinical testing of a medicament, the method comprising the following steps.

identifying a first population of individuals whose DNA encodes a GHRd3 polypeptide and a second population of individuals whose DNA does not encode a GHRd3 polypeptide;

administering a medicament to individuals of said first and/or said second population of individuals. In one embodiment, the medicament is administered to individuals of said first population but not to individuals of said second population. In one embodiment, the medicament is administered to individuals of said second population but not to individuals of said first population. In another embodiment, the medicament is administered to the individuals of both said first and said second populations.

The medicament according to the preceding methods is preferably a medicament for the treatment of short stature, obesity, infection, or diabetes; acromegaly or gigantism conditions which could be associated with lactogenic, diabetogenic, lipolytic and protein anabolic effects; conditions associated with sodium and water retention; metabolic syndromes; mood and sleep disorders, cancer, cardiac disease and hypertension.

A preferred aspect of the invention relates to a method for the clinical testing of a medicament, preferably a medicament capable of increasing the growth rate of a human subject. The method comprises the following steps.

administering a medicament, preferably a medicament capable increasing the growth rate of a human subject, to a population of individuals; and

from said population, identifying a first subpopulation of individuals whose DNA encodes a GHRd3 polypeptide isoform and a second subpopulation of individuals whose DNA does not encode a GHRd3 polypeptide isoform.

Another preferred aspect concerns a method for the clinical testing of a medicament, preferably a medicament capable of increasing the growth rate of a human subject or capable of ameliorating ISS, IUGR or SGA. The method comprises the following steps:

identifying a first population of individuals whose DNA encodes a GHRd3 polypeptide and a second population of individuals whose DNA does not encode a GHRd3 polypeptide; and

administering a medicament, preferably a medicament capable of preferably a medicament capable increasing the growth rate of a human subject or capable of ameliorating ISS, IUGR or SGA, to individuals of said first and/or said second population of individuals. In one embodiment, the medicament is administered to individuals of said first population but not to individuals of said second population. In one embodiment, the medicament is administered to individuals of said second population but not to individuals of said first population. In another embodiment, the medicament is administered to the individuals of both said first and said second populations.

Assessing the response to a medicament capable of increasing the growth rate of a human subject or capable of ameliorating ISS, IuGR or SGA comprises assessing the change in height of an individual. Increasing the growth rate of a human subject includes not only the situation where the subject attains at least the same ultimate height as GH-deficient subjects treated with GH (i.e., subjects diagnosed with GHD), but also refers to a situation where the subject catches up in height at the same growth rate as GH-deficient subjects treated with GH, or achieves adult height that is within the target height range, i.e., an ultimate height consistent with their genetic potential as determined by the mid-parental target height.

In one aspect of any of the methods of the invention, the step of determining whether the DNA of subject encodes a particular GHR polypeptide isoform can be performed using a nucleic acid molecule that specifically binds a GHR nucleic acid molecule. In another aspect, the step of determining whether the DNA of subject encodes a GHR polypeptide isoform is performed using a nucleic acid molecule that specifically binds a GHR nucleic acid molecule. Preferably, the methods of the invention comprise determining whether the DNA of an individual encodes a GHRd3 protein or polypeptide. This may thus comprise determining whether the genomic DNA of an individual comprises a GHRd3 allele, whether mRNA obtained from an individual encodes a GHRd3 polypeptide, or whether the subject expresses a GHRd3 polypeptide.

For example, in any of the above embodiments, determining whether the DNA of an individual encodes a GHRd3 polypeptide may comprise the steps of:

a) providing a biological sample;

b) contacting said biological sample with:

ii) a polynucleotide that hybridizes under stringent conditions to a GHR, preferably a GHRd3, nucleic acid; or

iii) a detectable polypeptide that selectively binds to a GHR, preferably a GHRd3 polypeptide; and

c) detecting the presence or absence of hybridization between said polynucleotide and an RNA species within said sample, or the presence or absence of binding of said detectable polypeptide to a polypeptide within said sample.

Preferably the biological sample is contacted with a polynucleotide that hybridizes under stringent conditions to a GHRd3 nucleic acid or a detectable polypeptide that selectively binds to a GHRd3 polypeptide, wherein a detection of said hybridization or of said binding indicates that said GHRd3 is expressed within said sample.

Preferably, said polynucleotide is a primer, and wherein said hybridization is detected by detecting the presence of an amplification product comprising said primer sequence. Preferably, said detectable polypeptide is an antibody. Detecting the GHRd3 and GHRfl polypeptides or nucleic acids can be carried out by any suitable method. For example, a serum level of the extracellular domain of GHRd3 or GHRfl may be assessed (e.g. the high-affinity GH binding protein) can be assessed. Oligonucleotide probes or primers hybridizing specifically with a GHRd3 genomic or cDNA sequence are also part of the present invention, as well as DNA amplification and detection methods using said primers and probes.

The invention also concerns methods of identifying candidate modulators of a GHRd3 polypeptide. Such methods may be embodied for example as methods for identifying GHR agonists or inhibitors that are effective in individuals homozygous or heterozygous for the GHRd3 allele. The methods can be used to identify compounds from known GH compositions, for example GENOTROPIN™, PROTROPIN™, NUTROPIN™, SOMAVERT™ (pegvisomant), to identify compounds most effective for treatment. The methods can thus be useful for identifying medicaments capable of increasing the growth rate of a human subject, capable of ameliorating obesity, infection, or diabetes; acromegaly or gigantism conditions which could be associated with lactogenic, diabetogenic, lipolytic and protein anabolic effects; conditions associated with sodium and water retention; metabolic syndromes; mood and sleep disorders, cancer, cardiac disease and hypertension.

In one aspect the invention concerns a method of identifying a candidate modulator of a GHRd3 polypeptide, said method comprising:

a) providing a GHRd3 polypeptide;

b) contacting said mixture with a test compound; and

b) determining whether said compound selectively binds to said GHRd3 polypeptide;

wherein a detection that said compound selectively binds to said polypeptide indicates that said compound is a candidate modulator of said GHRd3 polypeptide. Preferably the test compound is a GH polypeptide or a portion or variant thereof. The compound may be an agonist or inhibitor of the GHRd3 polypeptide. In preferred embodiments, said GHRd3 polypeptide is incorporated into a membrane.

The invention also provides a method of identifying a candidate modulator of a GHRd3 polypeptide, said method comprising:

a) providing a GHRd3 polypeptide;

b) contacting said mixture with a test compound; and

c) determining whether said compound selectively modulates GHR activity;

wherein a detection that said compound selectively modulates GHR activity indicates that said compound is a candidate modulator of GHRd3 polypeptide activity. Preferably the test compound is a GH polypeptide or a portion or variant thereof. The compound may be an agonist or inhibitor of the GHRd3 polypeptide. A detection that the test compound stimulates GHR activity indicates that the test compound is a candidate agonist. A detection that the test compound inhibits GHR activity indicates that the test compound is a candidate inhibitor. In preferred embodiments, said GHRd3 polypeptide is incorporated into a membrane.

The invention also provides a method of identifying a candidate modulator of a GHRd3 polypeptide polypeptide, said method comprising:

a) providing a cell comprising a GHRd3 polypeptide;

b) contacting said cell with a test compound; and

c) determining whether said compound selectively modulates GHR activity;

wherein a detection that said compound selectively modulates GHR activity indicates that said compound is a candidate modulator of GHRd3 polypeptide activity. Preferably the test compound is a GH polypeptide or a portion or variant thereof. The compound may be an agonist or inhibitor of the GHRd3 polypeptide. A detection that the test compound stimulates GHR activity indicates that the test compound is a candidate agonist. A detection that the test compound inhibits GHR activity indicates that the test compound is a candidate inhibitor. Preferably said cell is a human 293 cell. In another aspect of said method, the cell is a Xenopus laevis oocyte, and step a) comprises introducing to said cell GHRd3 cRNA.

The invention also provides that GHR may exist as a GHRd3 and GHRfl heterodimer polypeptide. Thus, in another aspect, the invention also concerns methods of identifying candidate modulators of a GHR heterodimer (GHRd3/fl) polypeptide. Such methods may be embodied for example as methods for identifying GHR agonists or inhibitors. Such methods may also be embodied as methods for identifying medicaments capable of increasing the growth rate of a human subject, capable of ameliorating obesity, infection, or diabetes; acromegaly or gigantism conditions which could be associated with lactogenic, diabetogenic, lipolytic and protein anabolic effects; conditions associated with sodium and water retention; metabolic syndromes; mood and sleep disorders, cancer, cardiac disease and hypertension.

In one aspect the invention concerns a method of identifying a candidate modulator of a GHR heterodimer polypeptide, said method comprising:

c) admixing a GHRfl and a GHRd3 polypeptide;

d) contacting said mixture with a test compound; and

b) determining whether said compound selectively binds to a GHRfl or GHRd3 polypeptide;

wherein a detection that said compound selectively binds to said polypeptide indicates that said compound is a candidate modulator of said GHR heterodimer polypeptide. Preferably the test compound is a GH polypeptide or a portion or variant thereof. The compound may be an agonist or inhibitor of the GHR heterodimer. In preferred embodiments, said GHRfl and GHRd3 polypeptide are incorporated into a membrane.

The invention also provides a method of identifying a candidate modulator of a GHR heterodimer polypeptide, said method comprising:

c) admixing a GHRfl and a GHRd3 polypeptide;

d) contacting said mixture with a test compound; and

c) determining whether said compound selectively modulates GHR activity;

wherein a detection that said compound selectively modulates GHR activity indicates that said compound is a candidate modulator of GHR heterodimer activity. Preferably the test compound is a GH polypeptide or a portion or variant thereof. The compound may be an agonist or inhibitor of the GHR heterodimer. A detection that the test compound stimulates GHR activity indicates that the test compound is a candidate agonist. A detection that the test compound inhibits GHR activity indicates that the test compound is a candidate inhibitor. In preferred embodiments, said GHRfl and GHRd3 polypeptide are incorporated into a membrane.

The invention also provides a method of identifying a candidate modulator of a GHR heterodimer polypeptide, said method comprising:

c) providing a cell comprising a GHRfl and a GHRd3 polypeptide;

d) contacting said cell with a test compound; and

c) determining whether said compound selectively modulates GHR activity;

wherein a detection that said compound selectively modulates GHR activity indicates that said compound is a candidate modulator of GHR heterodimer activity. Preferably the test compound is a GH polypeptide or a portion or variant thereof. The compound may be an agonist or inhibitor of the GHR heterodimer. A detection that the test compound stimulates GHR activity indicates that the test compound is a candidate agonist. A detection that the test compound inhibits GHR activity indicates that the test compound is a candidate inhibitor. Preferably said cell is a human 293 cell. In another aspect of said method, the cell is a Xenopus laevis oocyte, and step a) comprises introducing to said cell GHRd3 cRNA.

Preferably the cell is a cell expressing a GHRfl and a GHRd3 polypeptide. In preferred aspects, step a) comprises introducing to said cell a nucleic acid comprising the GHRd3 nucleotide sequence and a nucleic acid comprising the GHRfl nucleotide sequence. In other aspects, step a) comprises introducing to a cell expressing a GHRfl nucleic acid a nucleic acid comprising the GHRd3 nucleotide sequence. In yet other aspects, step a) comprises introducing to a cell expressing a GHRd3 nucleic acid a nucleic acid comprising the GHRfl nucleotide sequence.

The invention also provides a recombinant vector comprising a polynucleotide encoding a GHRd3 and a GHRfl polynucleotide. Also encompassed is a host cell comprising a recombinant vector according to the invention. The invention also provides a set of at least two recombinant vectors, comprising a first recombinant vector comprising a GHRd3 polynucleotide and a second recombinant vector comprising a GHRfl polynucleotide. The invention also provides a host cell comprising said first and said second recombinant vector according, as well as a non-human host animal or mammal comprising said recombinant vectors.

The invention also provides a mammalian host cell comprising a GHR gene disrupted by homologous recombination with a knock out vector comprising a GHRd3 polynucleotide. The invention further provides a non-human host mammal comprising a GHR gene disrupted by homologous recombination with a knock out vector comprising a GHRd3 polynucleotide.

As discussed, it will be appreciated that said methods of performing assays, methods identifying modulators of a GHR heterodimer, recombinant vector, host cells and non-human host mammal may employ a GHRd3 allele having a deletion of substantially the entire exon 3, or may instead employ any suitable GHR allele or isoform encoded by a GHR nucleic acid having a deletion, insertion or subsitution of one or more nucleic acids in exon 3.

### BRIEF DESCRIPTION OF THE DRAWINGS

FIG. 1 shows a cDNA sequence (SEQ ID NO: 1) encoding the GHRfl isoform.

FIG. 2 shows the amino acid sequence (SEQ ID NOS: 2 and 3) of the GHRfl isoform.

FIG. 3 shows the genomic DNA sequence (SEQ ID NO: 4) surrounding exon 3 of the human GHR gene (Genbank accession number AF155912).

FIG. 4 shows the genomic DNA sequence (SEQ ID NO: 6) surrounding the deleted exon 3 of the GHRd3 allele of the human GHR gene (Genbank accession number AF210633).

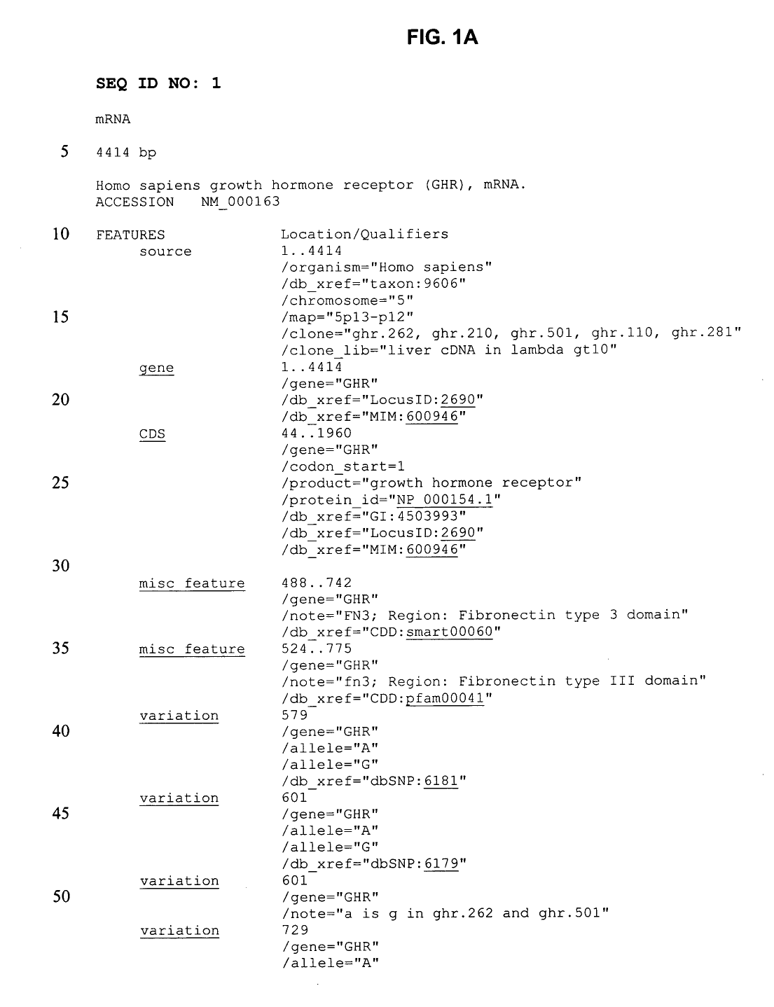

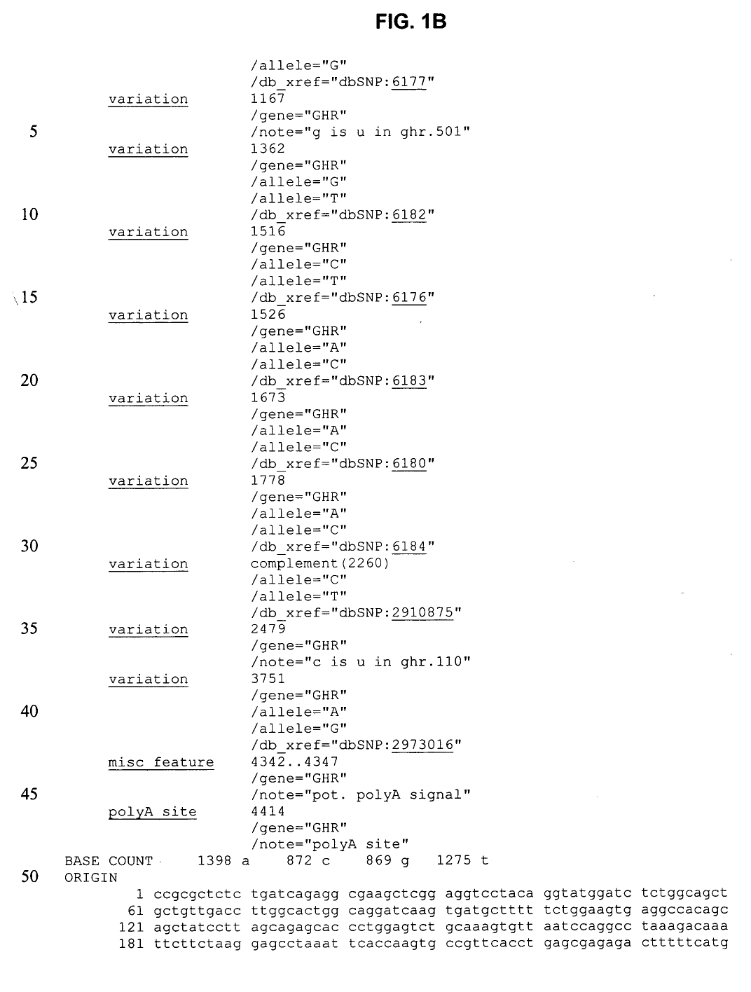

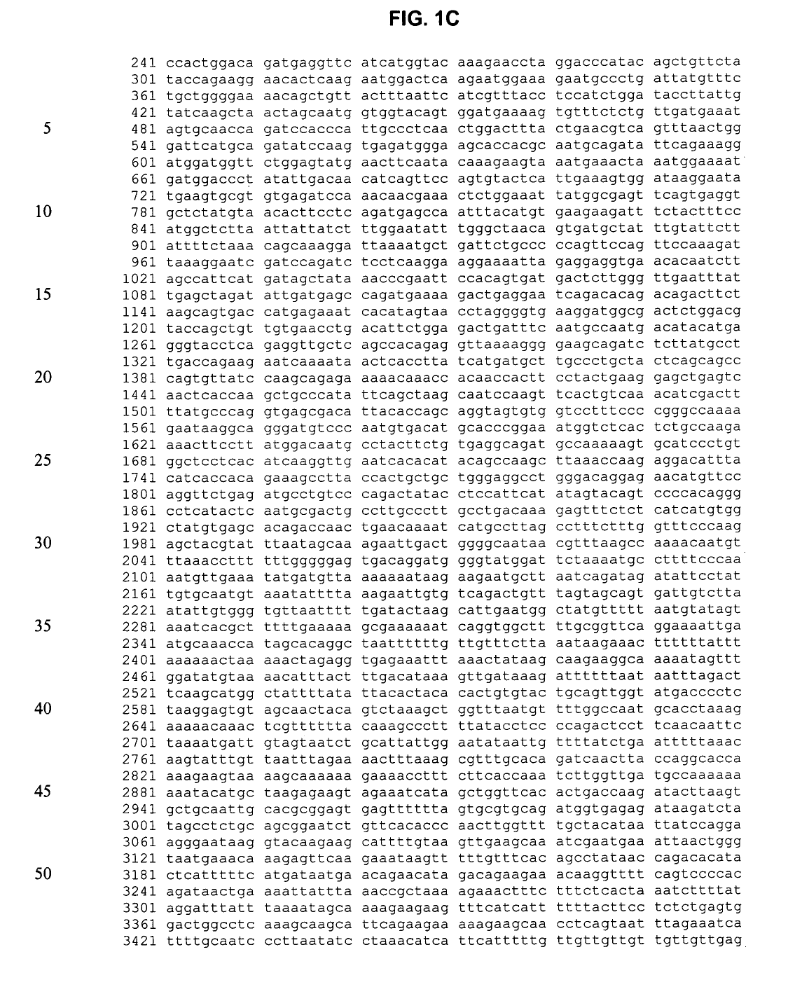

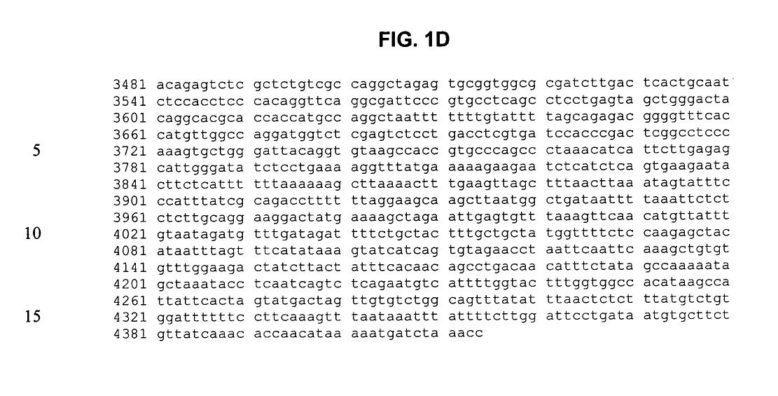

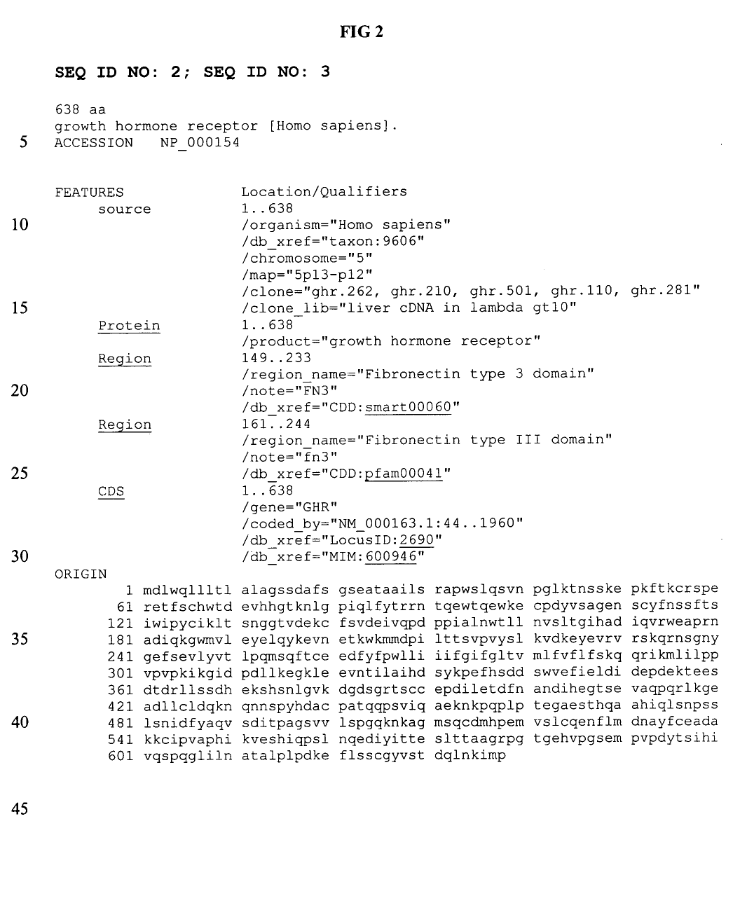

### DETAILED DESCRIPTION

97 Children with idiopathic short stature (ISS) who had been enrolled in trials for treatment with recombinant GH were examined for association of the common GHR exon 3 variant and the response of growth velocity to treatment with GH. The GHRd3 allele was present in 47 patients, of which 3 were GHRd3/d3 homozygotes and 44 were GHRd3/fl heterozyotes. After adjustment for age, sex, dose of rGH, it was found that children who carried the GHRd3 variant grew at a superior rate when treated with rGH. Growth velocity was 9.0+/−0.3 cm/yr the first year of therapy and 7.8+/−0.2 cm/yr the second year in children with GHRd3/fl or GHRd3/d3 genotypes, compared with 7.4+/−0.2 and 6.5+/−0.2 cm/yr, respectively, in children with GHRfl/fl genotypes (P<0.0001). The genotypic groups were comparable with respect to other medical and therapeutic characteristics. The genomic variation of the GHR sequence is therefore associated with a marked difference in rGH efficiency.

As discussed above, the present invention pertains to the field of pharmacogenomics and predictive medicine in which diagnostic assays, prognostic assays, and monitoring clinical trials are used for prognostic (predictive) purposes to thereby treat an individual. Accordingly, one aspect of the present invention relates to diagnostic assays for determining GHR protein and/or nucleic acid expression, in the context of a biological sample (e.g., blood, serum, cells, tissue) to thereby determine the nature of an individual's GHR response, particularly to treatment with an exogenous GH composition. This may be useful also to detect whether an individual is afflicted with a disease or disorder, or is at risk of developing a disorder, associated with diminished GHR response or activity. Disorders or conditions involving GHR activity include short stature, obesity, infection, or diabetes; acromegaly or gigantism conditions which could be associated with lactogenic, diabetogenic, lipolytic and protein anabolic effects; conditions associated with sodium and water retention; metabolic syndromes; mood and sleep disorders, cancer, cardiac disease and hypertension. The invention also provides for prognostic (or predictive) assays for determining whether an individual is at risk of developing a disorder associated with GHR protein activity. For example, the GHRd3 and GHRfl isoforms can be assayed in a biological sample. Such assays can be used for prognostic or predictive purpose to thereby prophylactically treat an individual prior to the onset of a disorder characterized by or associated with diminished GHR response, for example by administration of an effective amount of GH so that a subject attains an ultimate height consistent with their genetic potential. In other aspects, the invention provides methods of detecting agents that modulate GHRd3/GHRfl heterodimer activity. Such agents may be useful in the treatment of the aforementioned conditions or disorders involving GHR activity.

Definitions

The term “agent” is used herein to denote a chemical compound, a mixture of chemical compounds, a biological macromolecule, preferably a peptide or protein, or an extract made from biological materials such as bacteria, plants, fungi, or animal (particularly mammalian) cells or tissues.

In the context of the present invention, a “positive response” or “positive therapeutic response” to a medicament or agent can be defined as comprising a reduction of the symptoms related to a disease or condition. For example, a positive response may be an increase in height or growth rate upon administration of an agent. In the context of the present invention, a “negative response” to a medicament can be defined as comprising either a lack of positive response to the medicament, or which leads to a side-effect observed following administration of a medicament.

The term “polypeptide” refers to a polymer of amino acids without regard to the length of the polymer; thus, peptides, oligopeptides, and proteins are included within the definition of polypeptide. This term also does not specify or exclude post-expression modifications of polypeptides, for example, polypeptides which include the covalent attachment of glycosyl groups, acetyl groups, phosphate groups, lipid groups and the like are expressly encompassed by the term polypeptide. Also included within the definition are polypeptides which contain one or more analogs of an amino acid (including, for example, non-naturally occurring amino acids, amino acids which only occur naturally in an unrelated biological system, modified amino acids from mammalian systems etc.), polypeptides with substituted linkages, as well as other modifications known in the art, both naturally occurring and non-naturally occurring.

An “isolated” or “purified” protein or biologically active portion thereof is substantially free of cellular material or other contaminating proteins from the cell or tissue source from which the protein is derived, or substantially free from chemical precursors or other chemicals when chemically synthesized. The language “substantially free of cellular material” includes preparations of GH or GHR protein in which the protein is separated from cellular components of the cells from which it is isolated or recombinantly produced. In one embodiment, the language “substantially free of cellular material” includes preparations of GH or GHR protein having less than about 30% (by dry weight) of non-GH or non-GHR protein (also referred to herein as a “contaminating protein”), more preferably less than about 20% of non-GH or non-GHR protein, still more preferably less than about 10% of non-GH or non-GHR protein, and most preferably less than about 5% non-GH or non-GHR protein. When the GH or GHR protein or biologically active portion thereof is recombinantly produced, it is also preferably substantially free of culture medium, i.e., culture medium represents less than about 20%, more preferably less than about 10%, and most preferably less than about 5% of the volume of the protein preparation.

The language “substantially free of chemical precursors or other chemicals” includes preparations of GH or GHR protein in which the protein is separated from chemical precursors or other chemicals which are involved in the synthesis of the protein. In one embodiment, the language “substantially free of chemical precursors or other chemicals” includes preparations of GH or GHR protein having less than about 30% (by dry weight) of chemical precursors or non-GH or non-GHR chemicals, more preferably less than about 20% chemical precursors or non-GH or non-GHR chemicals, still more preferably less than about 10% chemical precursors or non-GH or non-GHR chemicals, and most preferably less than about 5% chemical precursors or non-GH or non-GHR chemicals.

The term “recombinant polypeptide” is used herein to refer to polypeptides that have been artificially designed and which comprise at least two polypeptide sequences that are not found as contiguous polypeptide sequences in their initial natural environment, or to refer to polypeptides which have been expressed from a recombinant polynucleotide.

A nucleic acid is “operably linked” when it is placed into a functional relationship with another nucleic acid sequence. For instance, a promoter or enhancer is operably linked to a coding sequence if it affects the transcription of the sequence. With respect to transcription regulatory sequences, operably linked means that the DNA sequences being linked are contiguous and, where necessary to join two protein coding regions, contiguous and in reading frame.

The term “primer” denotes a specific oligonucleotide sequence which is complementary to a target nucleotide sequence and used to hybridize to the target nucleotide sequence. A primer serves as an initiation point for nucleotide polymerization catalyzed by either DNA polymerase, RNA polymerase or reverse transcriptase.

The term “probe” denotes a defined nucleic acid segment (or nucleotide analog segment, e.g., polynucleotide as defined herein) which can be used to identify a specific polynucleotide sequence present in samples, said nucleic acid segment comprising a nucleotide sequence complementary of the specific polynucleotide sequence to be identified.

As used herein, a “test sample” refers to a biological sample obtained from a subject of interest. For example, a test sample can be a biological fluid (e.g., serum), cell sample, or tissue.

The terms “trait” and “phenotype” are used interchangeably herein and refer to any clinically distinguishable, detectable or otherwise measurable property of an organism such as symptoms of, or susceptibility to a disease for example. Typically the terms “trait” or “phenotype” are used herein to refer to an individual's response to an agent acting on GHR.

The term “genotype” as used herein refers the identity of the alleles present in an individual or a sample. In the context of the present invention a genotype preferably refers to the description of the alleles present in an individual or a sample. The term “genotyping” a sample or an individual for an allele involves determining the specific allele carried by an individual.

The term “allele” is used herein to refer to a variant of a nucleotide sequence. For example, alleles of the GHR nucleotide sequence include GHRd3 and GHRfl.

As used herein, “isoform” and “GHR isoform” refer to a polypeptide that is encoded by at least one exon of the GHR gene. Examples of a GHR isoform include GHRd3 and GHRfl polypeptides.

The term “polymorphism” as used herein refers to the occurrence of two or more alternative genomic sequences or alleles between or among different genomes or individuals. “Polymorphic” refers to the condition in which two or more variants of a specific genomic sequence can be found in a population.

A “polymorphic site” is the locus at which the variation occurs. A polymorphism may comprise a substitution, deletion or insertion of one or more nucleotides. A single nucleotide polymorphism is a single base pair change.

As used herein, “exon” refers to any segment of an interrupted gene that is represented in the mature RNA product.

As used herein, “intron” refers to a segment of an interrupted gene that is not represented in the mature RNA product. Introns are part of the primary nuclear transcript but are spliced out to produce mRNA, which is then transported to the cytoplasm.

As used herein, “growth hormone” or “GH” refers to growth hormone in native-sequence or in variant form, and from any source, whether natural, synthetic, or recombinant. Examples include but are not limited to human growth hormone (hGH), which is natural or recombinant GH with the human native sequence (for example, GENOTROPIM™, somatotropin or somatropin), and recombinant growth hormone (rGH), which refers to any GH or GH variant produced by means of recombinant DNA technology, including somatrem, somatotropin, somatropin and pegvisomant. A GH molecule may be an agonist or antagonist at the GHR.

As used herein, “growth hormone receptor” or “GHR” refers to the growth hormone receptor in native-sequence or in variant form, and from any source, whether natural, synthetic, or recombinant. The term “GHR” encompasses the GHRfl as well as the GHRd3 isoforms. Examples include human growth hormone receptor (hGHR), which is natural or recombinant GHR with the human native sequence. As used herein “GHRd3” refers to an exon 3-deleted isoform of GHR. The term “GHRfl” refers to an exon 3-containing GHR isoform. The term GHRd3 includes but is not limited to the polypeptide described in Urbanek M et al, Mol Endocrinol 1992 Feb;6(2):279-87, incorporated herein by reference. The terms GHRfl includes but is not limited to the polypeptide described in Leung et al., Nature, 330: 537-543 (1987), incorporated herein by reference.

The term “GHR gene”, when used herein, encompasses genomic, mRNA and cDNA sequences encoding any GHR protein, including the untranslated regulatory regions of the genomic DNA. The term “GHR gene” also encompasses alleles of the GHR gene, such as the GHRd3 allele and the GHRfl allele.

The Human GHR Gene and Protein

The human GHR gene is a single copy gene that spans 90 kb of the 5p13-12 chromosomal region. It contains nine coding exons (numbered 2-10) and several untranslated exons: exon 2 codes for the signal peptide, exons 3 to 7 encode the extracellular domain, exon 8 encodes the transmembrane domain and exons 9 and 10 encode the cytoplasmic domain. As discussed above, the hGHRd3 protein differs from the hepatic hGHR by a deletion of 22 amino acids within the extracellular domain of the receptor Godowski et al (1989). Genbank accession number AF155912, the disclosure of which sequence is incorporated herein by reference, provides the nucleotide sequence of the genomic DNA region surrounding exon 3 of the GHR gene (e.g. GHRfl allele). This 6.8 bp fragment comprising exon 3 and a portion of introns 2 and 3 also comprises two 251 bp repeat elements. These repeat elements flank exon 3, with the 5′ and 3′ repeated elements located 577 bp upstream and 1821 bp downstream of the exon. The elements are composed of a 171 bp long terminal repeat (LTR) fragment from a human endogenous retrovirus which belongs to the HERV-P family (Boeke, J. D., and Stoye, J. P. (1997) in Retroviruses (Coffin, J. M., Hughes, S. H., and Varmus, H. E., eds), pp. 343-435, Cold Spring Harbor Laboratory, Cold Spring Harbor, N.Y.). The LTR is followed by a 80 bp from a medium reiteration frequency MER4-type sequence (Smit, A. F. (1996) Curr. Opin. Genet. Dev. 6, 743-748). The seqence of the two 251 bp-long copies referred to as 5′ and 3′ repeat are 99% identical, differing in only three nucleotides at position 14, 245 and 246 of the repeat. In particular, as reported by Pantel et al (2000), the element located upstream from exon 3 caries a cytosine at position 14 and a thymine at positions 245 and 245, whereas the element located downstream of exon 3 carries a guanine, a cytosine and an adenine at these positions. Futhermore, other sequences of viral origin are found flanking exon 3.

The GHRd3 allele comprises a deletion of exon 3 and surrounding portions of introns 2 and 3. Unlike the GHRfl allele, the GHRd3 allele contains a single 251 bp LTR which is identical in sequence to the LTR element to te 3′ copy identidied on GHRfl alleles. The genomic DNA sequence of the GHRd3 allele in the region of the deleted exon 3 is shown in Genbank accession number AF210633, the disclosure of which sequence is incorporated herein by reference. Based on the GHRd3 and GHRfl sequence, known methods for detecting GHR nucleic acids or polypeptides can be used to determine whether an individual carries a GHRd3 allele.

The GHRd3 protein containing a deletion of exon 3 differs from the full length hGHR (GHRfl) by a deletion of 22 amino acids within the extracellular domain of the receptor. Any known method can thus be used to detect the presence of a GHRd3 or GHRfl protein. GHRd3 and GHRfl may also be detected in their untruncated form, or in truncated form, as a “high-affinity growth hormone binding protein”, “high-affinity GHBP” or “GHBP”, referring to the extracellular domain of the GHR that circulates in blood and functions as a GHBP in several species (Ymer and Herington, (1985) Mol. Cell. Endocrinol. 41: 153; Smith and Talamantes, (1988) Endocrinology, 123: 1489-1494; Emtner and Roos, Acta Endocrinologica (Copenh.), 122: 296-302 (1990), including man. Baumann et al., J. Clin. Endocrinol. Metab., 62: 134-141 (1986); EP 366,710; Herington et al., J. Clin. Invest., 77: 1817-1823 (1986); Leung et al., Nature, 330: 537-543 (1987). Various methods exist for measuring functional GHBP in serum are available, with the preferred method being a ligand-mediated immunofunctional assay (LIFA) described in U.S. Pat. No. 5,210,017 and further herein.

### GRd3 in Diagnostics, Therapy and Pharmacogenetics

In preferred embodiments, the invention involves determining whether a subject expresses a GHR allele associated with an increased or decreased response to treatment or with an increased or decreased GHR activity. Determining whether a subject expresses a GHR allele can be carried out by detecting a GHR protein or nucleic acid.

Preferably, the methods of treating, diagnosing or assessing a subject comprise assessing or determining whether a subject expresses a GHRd3 and/or GHRfl allele, e.g. determining whether a subject is a homozygote for the GHRfl allele (GHRfl/fl), a bomozygote for the GHRd3 allele (GHRd3/d3), or a heterozygote (GHRd3/fl). The invention thus preferably involves determining whether a GHRd3 is expressed within a biological sample comprising: a) contacting said biological sample with: i) a polynucleotide that hybridizes under stringent conditions to a GHRd3 nucleic acid; or ii) a detectable polypeptide that selectively binds to a GHRd3 polypeptide; and b) detecting the presence or absence of hybridization between said polynucleotide and an RNA species within said sample, or the presence or absence of binding of said detectable polypeptide to a polypeptide within said sample. A detection of said hybridization or of said binding indicates that said GHRd3 allele or isoform is expressed within said sample. Preferably, the polynucleotide is a primer, and wherein said hybridization is detected by detecting the presence of an amplification product comprising said primer sequence, or the detectable polypeptide is an antibody.

Also envisioned is a method of determining whether a mammal, preferably human, has an elevated or reduced level of GHRd3 expression, comprising: a) providing a biological sample from said mammal; and b) comparing the amount of a GHRd3 polypeptide or of a GHRd3 RNA species encoding a GHRd3 polypeptide within said biological sample with a level detected in or expected from a control sample. An increased amount of said GHRd3 polypeptide or said GHRd3 RNA species within said biological sample compared to said level detected in or expected from said control sample indicates that said mammal has an elevated level of GHRd3 expression, and wherein a decreased amount of said GHRd3 polypeptide or said GHRd3 RNA species within said biological sample compared to said level detected in or expected from said control sample indicates that said mammal has a reduced level of GHRd3 expression.

An exemplary method for detecting the presence or absence of the GHRd3 protein or nucleic acid in a biological sample involves obtaining a biological sample from a test subject and contacting the biological sample with a compound or an agent capable of detecting GHRd3 protein or nucleic acid (e.g., mRNA, genomic DNA) that encodes GHRd3 protein such that the presence of GHRd3 protein or nucleic acid is detected in the biological sample. A preferred agent for detecting GHRd3 MRNA or genomic DNA is a labeled nucleic acid probe capable of hybridizing to GHRd3 mRNA or genomic DNA. The nucleic acid probe can be, for example, a human nucleic acid, or a portion thereof, such as an oligonucleotide of at least 15, 30, 50, 100, 250 or 500 nucleotides in length and sufficient to specifically hybridize under stringent conditions to GHRd3 MRNA or genomic DNA. Other suitable probes for use in the diagnostic assays of the invention are described herein.

A preferred agent for detecting the GHRd3 protein is an antibody capable of binding to the GHRd3 protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin.

The term “biological sample” is intended to include tissues, cells and biological fluids isolated from a subject, as well as tissues, cells and fluids present within a subject. That is, the detection method of the invention can be used to detect candidate mRNA, protein, or genomic DNA in a biological sample in vitro as well as in vivo. For example, in vitro techniques for detection of candidate mRNA include Northern hybridizations and in situ hybridizations. In vitro techniques for detection of the candidate protein include enzyme linked immunosorbent assays (ELISAs), Western blots, immunoprecipitations and immunofluorescence. In vitro techniques for detection of candidate genomic DNA include Southern hybridizations. Furthermore, in vivo techniques for detection of the GHRd3 protein include introducing into a subject a labeled anti-antibody. For example, the antibody can be labeled with a radioactive marker whose presence and location in a subject can be detected by standard imaging techniques.

In one embodiment, the biological sample contains protein molecules from the test subject. Alternatively, the biological sample can contain MRNA molecules from the test subject or genomic DNA molecules from the test subject. A preferred biological sample is a serum sample isolated by conventional means from a subject.

In another embodiment, the methods further involve obtaining a control biological sample from a control subject, contacting the control sample with a compound or agent capable of detecting the GHRd3 protein, MRNA, or genomic DNA, such that the presence of GHRd3 protein, mRNA or genomic DNA is detected in the biological sample, and comparing the presence of GHRd3 protein, mRNA or genomic DNA in the control sample with the presence of GHRd3 protein, MRNA or genomic DNA in the test sample.

The invention also encompasses kits for detecting the presence of the GHRd3 protein, mRNA, or genomic DNA in a biological sample. For example, the kit can comprise a labeled compound or agent capable of detecting GHRd3 protein or mRNA in a biological sample; means for determining the amount of GHRd3 protein or mRNA in the sample; and means for comparing the amount of GHRd3 protein, mRNA, or genomic DNA in the sample with a standard. The compound or agent can be packaged in a suitable container. The kit can further comprise instructions for using the kit to detect GHRd3 protein or nucleic acid.

Most preferably, the assays described herein, such as the preceding diagnostic assays or the following assays, can be utilized to identify a subject having or at risk of developing diminished GHR response. In particular, a GHRfl homozygote subject is identified as having or at risk of developing a diminished GHR response. In other aspects, the diagnostic methods described herein may be utilized to identify subjects having or at risk of developing a disease, disorder or trait associated with aberrant or more particularly decreased GHR levels, expression or activity. For example, the assays described herein, such as the preceding diagnostic assays or the following assays, can be utilized to identify a subject having or at risk of developing a trait associated with decreased GHR levels, expression or activity. In another example, the assays described herein can be utilized to identify a subject having or at risk of developing a trait associated with decreased GHR levels, expression or activity. As discussed, a GHRd3/fl heterozygote is expected to have increased GHR response or GHR activity compared to a GHRfl/fl homozygote.

The prognostic assays described herein can be used to determine whether and/or according to which administration regimen a subject is to be administered an agent which acts through the GHR pathway to treat a disease or disorder. Thus, the present invention provides methods for determining whether a subject can be effectively treated with an agent which acts through the GHR pathway in which a test sample is obtained and GHRd3 protein or nucleic acid expression or activity is detected. As discussed, a subject displaying the GHRd3 protein or nucleic acid is expected to have an increased positive response to said agent relative to a subject not displaying the GHRd3 protein or nucleic acid.

In large part because the administration of agents that act through GHR-mediated pathways can be adapted to subjects having higher or lower responsiveness to the agent, the detection of susceptibility to diminished GHR activity in individuals is very important. Said agents need not necessarily act directly on the GHR protein, but may act upstream of the GHR protein, for example acting on another molecule which ultimately interacts with the GHR protein. In a preferred embodiment, the agent is an agent that acts directly on the GHR protein. Most preferably, the agent is an agent that binds the GHR. protein and acts either as an agonist or an antagonist. Most preferably the agent is a GH protein capable of activation the GHR protein. In other embodiments, the agent is a GH protein capable of binding but not activating the GHR protein.

Disorders involving GHR include for example short stature, obesity, infection, or diabetes; acromegaly or gigantism conditions which could be associated with lactogenic, diabetogenic, lipolytic and protein anabolic effects; conditions associated with sodium and water retention; metabolic syndromes; mood and sleep disorders, cancer, cardiac disease and hypertension. Thus, the methods of the invention may be used to predict a subject's respose to treatment with an agent for any one of these disorders.

As discussed, the invention discloses a method for treating a subject suffering from a condition selected from the group consisting of short stature, obesity, infection, or diabetes; acromegaly or gigantism conditions which could be associated with lactogenic, diabetogenic, lipolytic and protein anabolic effects; conditions associated with sodium and water retention; metabolic syndromes; mood and sleep disorders, cancer, cardiac disease and hypertension, the method comprising:

(a) determining in the subject the presence or absence of an allele of the GHR gene, wherein the allele is correlated with a likelihood of having an increased or decreased positive response to an agent capable of ameliorating said condition; and

(b) selecting or determining an effective amount of said agent to administer to said subject.

Consequently, the invention also concerns a method for the treatment of a mammal, preferably a human, comprising the following steps:

optionally, determining whether the DNA of an individual encodes a GHRd3 protein;

selecting an individual whose DNA does not encode a GHRd3 protein;

following up said individual for the appearance (and optionally the development) of symptoms related to diminished GHR response; and

administering an effective amount of a treatment acting against diminished GHR response or against symptoms thereof to said individual at an appropriate stage.

Another embodiment of the present invention comprises a method for the treatment of a mammal, preferably a human, comprising the following steps.

optionally, determining whether the DNA of an individual encodes a GHRd3 protein;

selecting an individual whose DNA does not encode a GHRd3 protein;

administering a preventive treatment for diminished GHR response to said individual.

In a further embodiment, the present invention concerns a method for the treatment of a mammal, preferably a human, comprising the following steps.

optionally, determining whether the DNA of an individual encodes a GHRd3 protein;

selecting an individual whose DNA does not encode a GHRd3 protein;

administering a preventive treatment for diminished GHR response to said individual;

following up said individual for the appearance and the development of symptoms related to diminished GHR response; and optionally

administering a treatment acting against diminished GHR response or against symptoms thereof to said individual at the appropriate stage.

For use in the determination of the course of treatment of an individual, the present invention also concerns a method of treatment comprising the following steps.

selecting an individual whose DNA encodes a protein associated with diminished GHR response, activity or expression or of the symptoms thereof; and

administering a treatment effective against diminished GHR response or symptoms thereof to said individual. In preferred embodiments, said protein associated with diminished GHR response or of the symptoms thereof is a GHR protein, more preferably a GHRfl protein. Most preferably the individual will be homozygous for the GHRfl/fl isoform.

The individual according to the methods of the invention may be an individual suffering from or susceptible to a condition selected from the group consiting of: short stature (e.g. preferably ISS), obesity, infection, or diabetes; acromegaly or gigantism conditions which could be associated with lactogenic, diabetogenic, lipolytic and protein anabolic effects; conditions associated with sodium and water retention; metabolic syndromes; mood and sleep disorders, cancer, cardiac disease and hypertension.

The diminished GHR response is preferably a diminished response to treatment with an agent capable of acting through the GHR pathway, or more preferably binding to the GHR protein. Preferred examples of agents include agents for the treatment of short stature, obesity, infection, or diabetes; acromegaly or gigantism conditions which could be associated with lactogenic, diabetogenic, lipolytic and protein anabolic effects; conditions associated with sodium and water retention; metabolic syndromes; mood and sleep disorders, cancer, cardiac disease and hypertension.

A treatment effective against diminished GHR response or symptoms thereof may differ in any suitable aspect from the treatment administered to individuals who do not have a diminished GHR response. In one aspect, the treatment differs in amount of an agent administered. In another aspect, the treatment differs in formulation. In another aspect, the time of method of administering the composition differs. In yet another aspect, an agent used in a treatment effective against diminished GHR response or symptoms thereof differs in structure from the agent used to treat the underlying conditions (e.g. short stature, obesity). In a preferred aspect, a composition comprising a GH protein, variant or fragment thereof is administered to an individual homozygous for GHRfl in a higher amount than that administered to an individual whose DNA encodes a GHRd3 protein.

Preferably said agent is a GH polypeptide or fragment thereof, and more preferably a recombinant GH polypeptide or fragment thereof, examples of which are further discussed herein. The recombinant GH polypeptide may be a GHR agonist (e.g. for increasing growth or treating obesity) or a GHR antagonist (e.g. for the treatment of acromegaly or gigantism conditions). The response, as further discussed herein, may be change in height or growth rate, amelioration of symptoms of obesity (for example body mass index (BMI)), infection, or diabetes; amelioration of symptoms of acromegaly or gigantism conditions; or amelioration of symptoms of conditions associated with sodium and water retention, metabolic syndromes, mood and sleep disorders, cancer, cardiac disease and hypertension.

In one aspect, the individual may already be suffering from or be susceptible to a disorder and may already have been treated, may be undergoing therapy, or may be a candidate for future therapy. Most preferably, the individual will be suffering from of susceptible to a conditions selected from the group consisting of: short stature, obesity, infection, or diabetes; acromegaly or gigantism conditions which could be associated with lactogenic, diabetogenic, lipolytic and protein anabolic effects; conditions associated with sodium and water retention; metabolic syndromes; mood and sleep disorders, cancer, cardiac disease and hypertension. In preferred aspects, the invention thus provides methods for the treatment of individuals having one or more of said disorders. The present invention thus allows targeting for treatment according to a particular treatment method those subjects having diminished GH response as defined above.

A DNA sample is obtained from the individual to be tested to determine whether the DNA encodes a GHRd3 protein. The DNA sample is analyzed to determine whether it comprises the GHRd3 sequence or whether the individual is homozygous for the GHRfl isoform. DNA encoding a GHRd3 protein will be associated with a greater positive response to treatment with the medicament, and lack of DNA encoding GHRd3 alleles is associated with a diminished positive response when compared to GHRd3 individuals.

The methods of the invention can will also be useful in assessing and conducting clinical trials of medicaments. The methods accordingly comprise identifying a first population of individuals who respond positively to said medicament and a second population of individuals who respond negatively to said medicament or whose positive response to said medicament is diminished in comparison to said first population of individuals. In one embodiments, the medicament may be administered to the subject in a clinical trial if the DNA sample contains alleles of one or more alleles associated with a positive response to treatment with the medicament and/or if the DNA sample lacks alleles of one or more alleles associated with a negative or decreased positive response to treatment with the medicament. In another aspect, the medicament may be administered to the subject in a clinical trial if the DNA sample contains alleles of one or more alleles associated with a negative or decreased positive response to treatment with the medicament and/or if the DNA sample lacks alleles of one or more alleles associated with a positive or increased positive response to treatment with the medicament.

Thus, using the method of the present invention, drug efficacy can be assessed by taking account of differences in GHR response among drug trial subjects. If desired, a trial for evaluation of drug efficacy may be conducted in a population comprised substantially of individuals likely to respond favorably to the medicament, or in a population comprised substantially of individuals likely to respond less favorable to the medicament that another population. For example, a GH protein-containing composition may be evaluated in either a population of GHRd3 individuals or in a population of GHRfl/fl individuals. In another aspect, a medicament designed to treat individuals suffering from diminished GH response may be evaluated advantageously in a population of GHRfl/fl individuals.

Detecting GHRd3 and GHRfl

It is contemplated that other mutations in the GHR gene may be identified in accordance with the present invention by detecting a nucleotide change in particular nucleic acids (U.S. Pat. No. 4,988,617, incorporated herein by reference). A variety of different assays are contemplated in this regard, including but not limited to, fluorescent in situ hybridization (FISH; U.S. Pat. No. 5,633,365 and U.S. Pat. No. 5,665,549, each incorporated herein by reference), direct DNA sequencing, PFGE analysis, Southern or Northern blotting, single-stranded conformation analysis (SSCA), RNAse protection assay, allele-specific oligonucleotide (ASO e.g., U.S. Pat. No. 5,639,611), dot blot analysis denaturing gradient gel electrophoresis (e.g., U.S. Pat. No. 5,190,856 incorporated herein by reference). RFLP (e.g., U.S. Pat. No. 5,324,631 incorporated herein by reference) and PCR-SSCP. Methods for detecting and quantitating gene sequences in for example biological fluids are described in U.S. Pat. No. 5,496,699, incorporated herein by reference.

Primers and Probes

The term primer, as defined herein, is meant to encompass any nucleic acid that is capable of priming the synthesis of a nascent nucleic acid in a template-dependent process. Typically, primers are oligonucleotides from ten to twenty base pairs in length, but longer sequences can be employed. Primers may be provided in double-stranded or single-stranded form, although the single-stranded form is preferred. Probes are defined differently, although they may act as primers. Probes, while perhaps capable of priming, are designed to binding to the target DNA or RNA and need not be used in an amplification process.

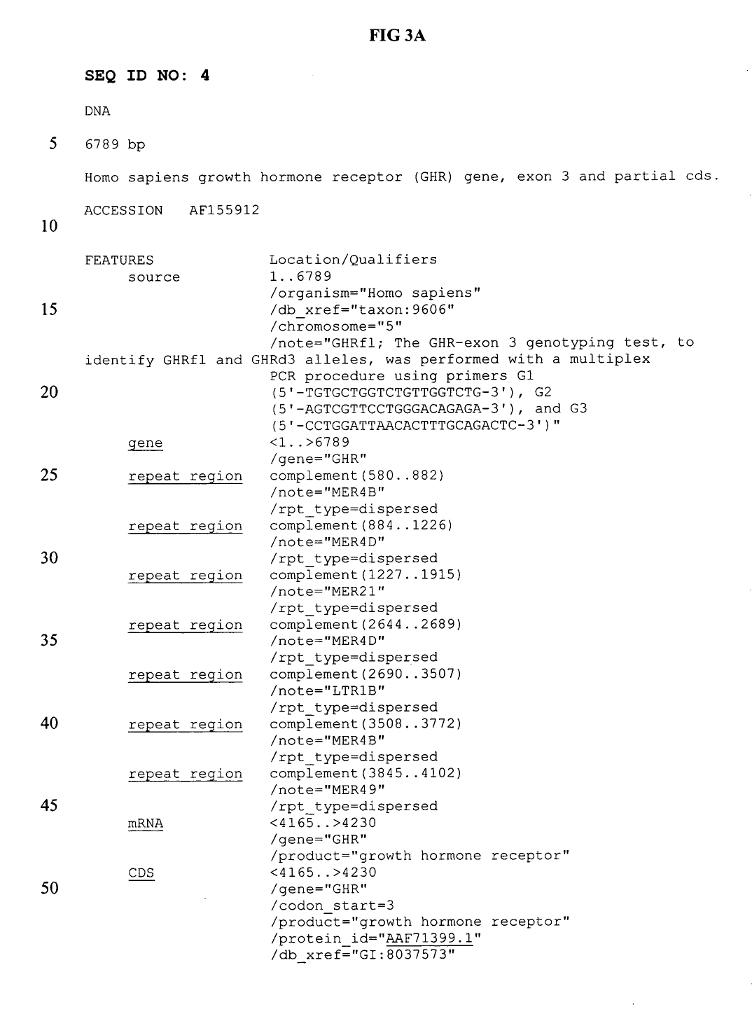

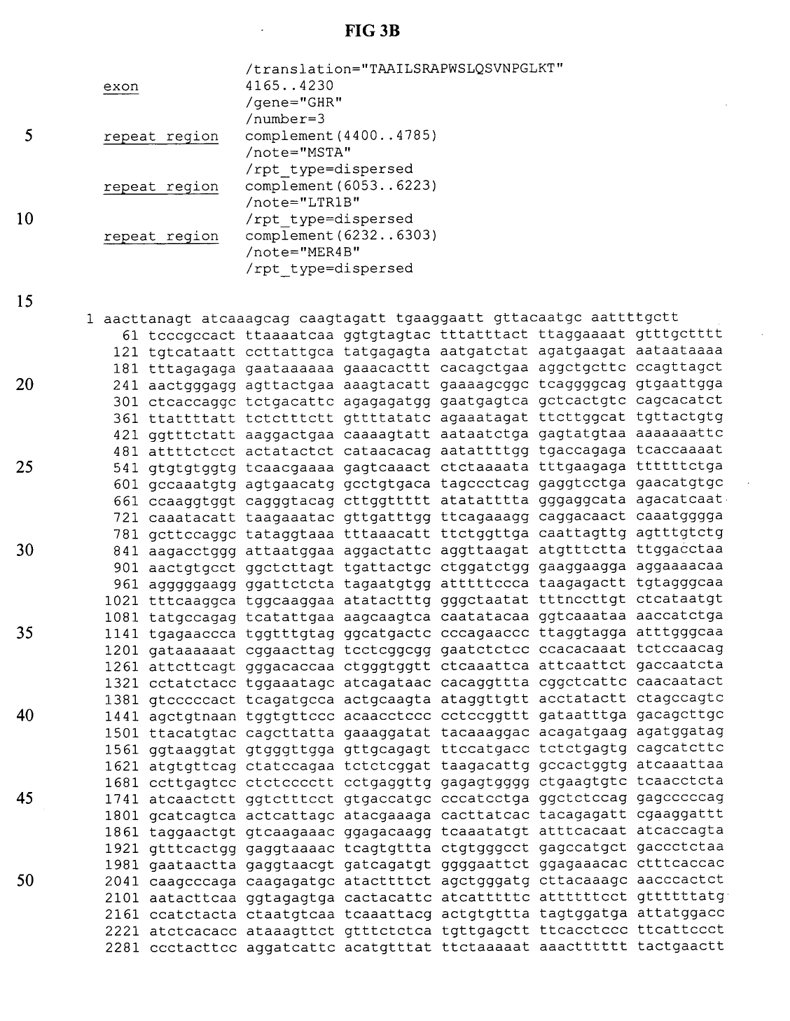

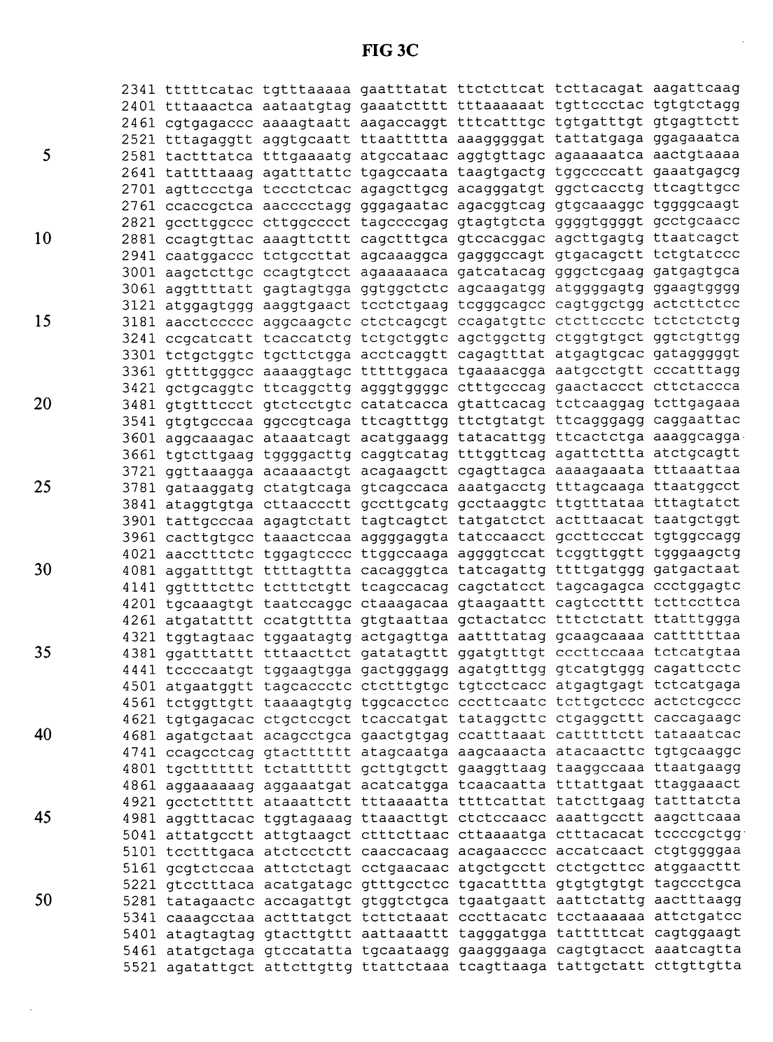

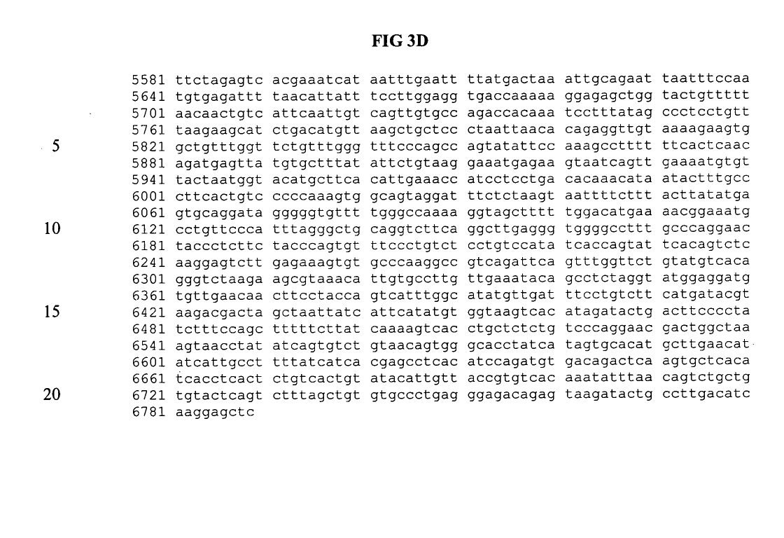

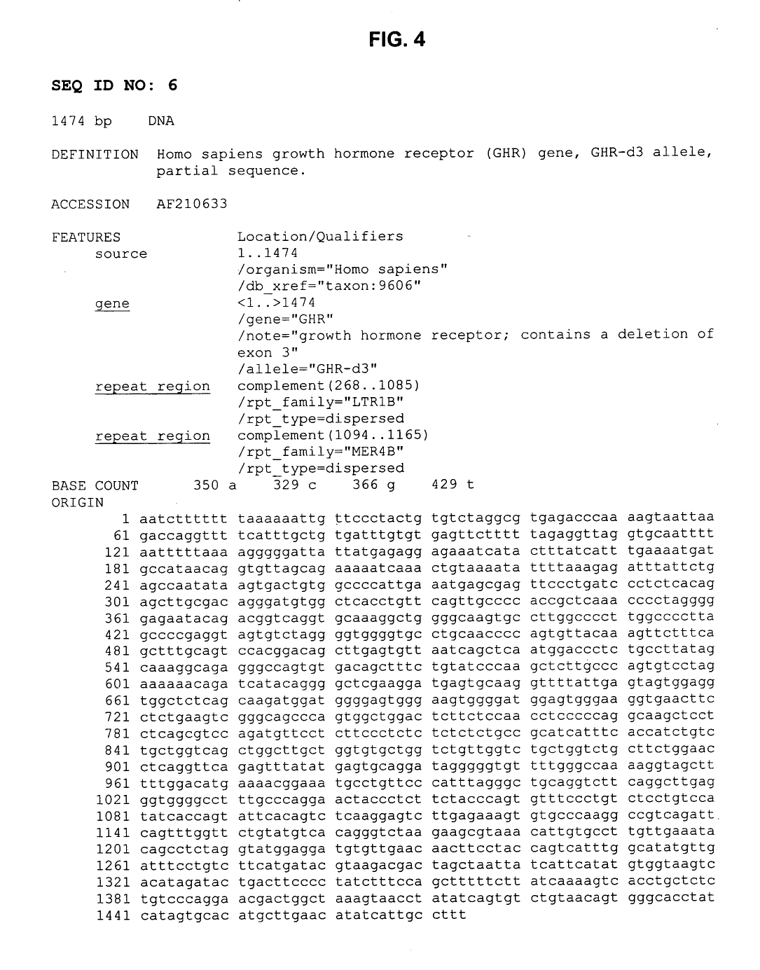

FIGS. 3 and 4 provide the genomic DNA sequences surrounding exon 3 or the site of the exon 3 deletion in the GHR gene, respectively. A GHRfl cDNA sequence is shown in SEQ ID NO 1. Any difference in nucleotide sequence between the GHRd3 and GHRfl alleles may be used in the methods of the invention in order to detect and distinuguish the particular GHR allele in an individual. To identify a GHRfl genomic DNA or cDNA molecule, a primer may be designed which hybridizes to an exon 3 nucleic acid. To identify a GHRd3 genomic DNA, a primer or probe may be designed such that it spans the junction of introns 2 and 3 of the GHR gene as found in the genomic DNA sequence of the GHRd3 allele, thereby distinguishing between the GHRfl allele which contains exon 3 and the GHRd3 allele which does not contain exon 3. In another example, a GHRd3 cDNA molecule may be identified by designing a primer or probe that spans the junction of exons 2 and 4, thereby distinguishing between an GHRfl cDNA molecule which contains exon 3 and a GHRd3 cDNA molecule which does not contain exon 3. Other examples of suitable primers for detection GHRd3 are listed in Pantel et al. (supra) and in Example 1 below.

The present invention encompasses polynucleotides for use as primers and probes in the methods of the invention. These polynucleotides may consist of, consist essentially of, or comprise a contiguous span of nucleotides of a sequence from any sequence provided herein as well as sequences which are complementary thereto (“complements thereof”). The “contiguous span” may be at least 25, 35, 40, 50, 70, 80, 100, 250, 500 or 1000 nucleotides in length, to the extent that a contiguous span of these lengths is consistent with the lengths of the particular Sequence ID. It should be noted that the polynucleotides of the present invention are not limited to having the exact flanking sequences surrounding a target sequence of interest, which are enumerated in the Sequence Listing. Rather, it will be appreciated that the flanking sequences surrounding the polymorphisms, or any of the primers of probes of the invention which, are more distant from the markers, may be lengthened or shortened to any extent compatible with their intended use and the present invention specifically contemplates such sequences. It will be appreciated that the polynucleotides referred to herein may be of any length compatible with their intended use. Also the flanking regions outside of the contiguous span need not be homologous to native flanking sequences which actually occur in human subjects. The addition of any nucleotide sequence, which is compatible with the nucleotides intended use is specifically contemplated. Preferred polynucleotides may consist of, consist essentially of, or comprise a contiguous span of nucleotides of a sequence from SEQ ID No 1, 4 or 6 as well as sequences which are complementary thereto. The “contiguous span” may be at least 8, 10, 12, 15, 50, 70, 80, 100, 250, 500 or 1000 nucleotides in length.

The probes of the present invention may be designed from the disclosed sequences for any method known in the art, particularly methods which allow for testing if a particular sequence or marker disclosed herein is present. A preferred set of probes may be designed for use in the hybridization assays of the invention in any manner known in the art such that they selectively bind to one allele of a polymorphism, but not the other under any particular set of assay conditions.

Any of the polynucleotides of the present invention can be labeled, if desired, by incorporating a label detectable by spectroscopic, photochemical, biochemical, immunochemical, or chemical means. For example, useful labels include radioactive substances, fluorescent dyes or biotin. Preferably, polynucleotides are labeled at their 3′ and 5′ ends. A label can also be used to capture the primer, so as to facilitate the immobilization of either the primer or a primer extension product, such as amplified DNA, on a solid support. A capture label is attached to the primers or probes and can be a specific binding member which forms a binding pair with the solid phase reagent's specific binding member (e. g. biotin and streptavidin). Therefore depending upon the type of label carried by a polynucleotide or a probe, it may be employed to capture or to detect the target DNA. Further, it will be understood that the polynucleotides, primers or probes provided herein, may, themselves, serve as the capture label. For example, in the case where a solid phase reagent's binding member is a nucleic acid sequence, it may be selected such that it binds a complementary portion of a primer or probe to thereby immobilize the primer or probe to the solid phase. In cases where a polynucleotide probe itself serves as the binding member, those skilled in the art will recognize that the probe will contain a sequence or “tail” that is not complementary to the target. In the case where a polynucleotide primer itself serves as the capture label, at least a portion of the primer will be free to hybridize with a nucleic acid on a solid phase. DNA Labeling techniques are well known to the skilled technician.

Any of the polynucleotides, primers and probes of the present invention can be conveniently immobilized on a solid support. Solid supports are known to those skilled in the art and include the walls of wells of a reaction tray, test tubes, polystyrene beads, magnetic beads, nitrocellulose strips, membranes, microparticles such as latex particles, sheep (or other animal) red blood cells, duracytes) and others. The solid support is not critical and can be selected by one skilled in the art. Thus, latex particles, microparticles, magnetic or non-magnetic beads, membranes, plastic tubes, walls of microtiter wells, glass or silicon chips, sheep (or other suitable animal's) red blood cells and duracytes are all suitable examples. Suitable methods for immobilizing nucleic acids on solid phases include ionic, hydrophobic, covalent interactions and the like. A solid support, as used herein, refers to any material which is insoluble, or can be made insoluble by a subsequent reaction. The solid support can be chosen for its intrinsic ability to attract and immobilize the capture reagent. Alternatively, the solid phase can retain an additional receptor which has the ability to attract and immobilize the capture reagent. The additional receptor can include a charged substance that is oppositely charged with respect to the capture reagent itself or to a charged substance conjugated to the capture reagent. As yet another alternative, the receptor molecule can be any specific binding member which is immobilized upon (attached to) the solid support and which has the ability to immobilize the capture reagent through a specific binding reaction. The receptor molecule enables the indirect binding of the capture reagent to a solid support material before the performance of the assay or during the performance of the assay. The solid phase thus can be a plastic, derivatized plastic, magnetic or non-magnetic metal, glass or silicon surface of a test tube, microtiter well, sheet, bead, microparticle, chip, sheep (or other suitable animal's) red blood cells, duracytes and other configurations known to those of ordinary skill in the art. The polynucleotides of the invention can be attached to or immobilized on a solid support individually or in groups of at least 2, 5, 8, 10, 12, 15, 20, or 25 distinct polynucleotides of the inventions to a single solid support. In addition, polynucleotides other than those of the invention may be attached to the same solid support as one or more polynucleotides of the invention.

Any polynucleotide provided herein may be attached in overlapping areas or at random locations on the solid support. Alternatively the polynucleotides of the invention may be attached in an ordered array wherein each polynucleotide is attached to a distinct region of the solid support which does not overlap with the attachment site of any other polynucleotide. Preferably, such an ordered array of polynucleotides is designed to be “addressable” where the distinct locations are recorded and can be accessed as part of an assay procedure. Addressable polynucleotide arrays typically comprise a plurality of different oligonucleotide probes that are coupled to a surface of a substrate in different known locations. The knowledge of the precise location of each polynucleotides location makes these “addressable” arrays particularly useful in hybridization assays. Any addressable array technology known in the art can be employed with the polynucleotides of the invention. One particular embodiment of these polynucleotide arrays is known as the Genechips, and has been generally described in U.S. Pat. No. 5,143,854; PCT publications WO 90/15070 and 92/10092. These arrays may generally be produced using mechanical synthesis methods or light directed synthesis methods, which incorporate a combination of photolithographic methods and solid phase oligonucleotide synthesis (Fodor et al., Science, 251: 767-777, 1991). The immobilization of arrays of oligonucleotides on solid supports has been rendered possible by the development of a technology generally identified as“Very Large Scale Immobilized Polymer Synthesis” (VLSIPS ) in which, typically, probes are immobilized in a high density array on a solid surface of a chip. Examples of VLSIPS technologies are provided in U.S. Pat. Nos. 5,143,854 and 5,412,087 and in PCT Publications WO 90/15070, WO 92/10092 and WO 95/11995, which describe methods for forming oligonucleotide arrays through techniques such as light directed synthesis techniques. In designing strategies aimed at providing arrays of nucleotides immobilized on solid supports, further presentation strategies were developed to order and display the oligonucleotide arrays on the chips in an attempt to maximize hybridization patterns and sequence information. Examples of such presentation strategies are disclosed in PCT Publications WO 94/12305, WO 94/11530, WO 97/29212 and WO 97/31256.

Template Dependent Amplification Methods

A number of template dependent processes are available to amplify the marker sequences present in a given template sample. One of the best known amplification methods is the polymerase chain reaction (referred to as PCR) which is described in detail in U.S. Pat. Nos. 4,683,195, 4,683,202 and 4,800,159, and in Innis et al., PCR Protocols, Academic Press, Inc. San Diego Calif., 1990., each of which is incorporated herein by reference in its entirety.

Briefly, in PCR, two primer sequences are prepared that are complementary to regions on opposite complementary strands of the marker sequence. An excess of deoxynucleoside triphosphates are added to a reaction mixture along with a DNA polymerase, e.g., Taq polymerase. If the marker sequence is present in a sample, the primers will bind to the marker and the polymerase will cause the primers to be extended along the marker sequence by adding on nucleotides. By raising and lowering the temperature of the reaction mixture, the extended primers will dissociate from the marker to form reaction products, excess primers will bind to the marker and to the reaction products and the process is repeated.

A reverse transcriptase PCR amplification procedure may be performed in order to quantify the amount of mRNA amplified. Methods of reverse transcribing RNA into cDNA are well known and described in Sambrook et al., In: Molecular Cloning. A Laboratory Manual. 2d Ed., Cold Spring Harbor Laboratory Press, Cold Spring Harbor, N.Y., 1989. Alternative methods for reverse transcription utilize thermostable, RNA-dependent DNA polymerases. These methods are described in WO 90/07641. Polymerase chain reaction methodologies are well known in the art.

Another method for amplification is the ligase chain reaction (“LCR” U.S. Pat. Nos. 5,494,810, 5,484,699, EPO No. 320 308, each incorporated herein by reference). In LCR, two complementary probe pairs are prepared, and in the presence of the target sequence, each pair will bind to opposite complementary strands of the target such that they abut. In the presence of a ligase, the two probe pairs will link to form a single unit.

By temperature cycling, as in PCR, bound ligated units dissociate from the target and then serve as “target sequences” for ligation of excess probe pairs. U.S. Pat. No. 4,883,750 describes a method similar to LCR for binding probe pairs to a target sequence.

Qbeta Replicase, an RNA-directed RNA polymerase, can be used as yet another amplification method in the present invention. In this method, a replicative sequence of RNA that has a region complementary to that of a target is added to a sample in the presence of an RNA polymerase. The polymerase will copy the replicative sequence that can then be detected. Similar methods also are described in U.S. Pat. No. 4,786,600, incorporated herein by reference, which concerns recombinant RNA molecules capable of serving as a template for the synthesis of complementary single-stranded molecules by RNA-directed RNA polymerase. The product molecules so formed also are capable of serving as a template for the synthesis of additional copies of the original recombinant RNA molecule.

An isothermal amplification method, in which restriction endonucleases and ligases are used to achieve the amplification of target molecules that contain nucleotide 5′-[alpha-thio]-triphosphates in one strand of a restriction site also may be useful in the amplification of nucleic acids in the present invention (Walker et al, (1992), Proc. Nat'l Acad Sci. USA, 89:392-396; U.S. Pat. No. 5,270,184 incorporated herein by reference). U.S. Pat. No. 5,747,255 (incorporated herein by reference) describes an isothermal amplification using cleavable oligonucleotides for polynucleotide detection. In the method described therein, separated populations of oligonucleotides are provided that contain complementary sequences to one another and that contain at least one scissile linkage which is cleaved whenever a perfectly matched duplex is formed containing the linkage. When a target polynucleotide contacts a first oligonucleotide cleavage occurs and a first fragment is produced which can hybridize with a second oligonucleotide. Upon such hybridization, the second oligonucleotide is cleaved releasing a second fragment that can in turn, hybridize with a first oligonucleotide in a manner similar to that of the target polynucleotide.

Strand Displacement Amplification (SDA) is another method of carrying out isothermal amplification of nucleic acids which involves multiple rounds of strand displacement and synthesis, i.e., nick translation (e.g., U.S. Pat. Nos. 5,744,311; 5,733,752; 5,733,733; 5,712,124). A similar method, called Repair Chain Reaction (RCR), involves annealing several probes throughout a region targeted for amplification, followed by a repair reaction in which only two of the four bases are present. The other two bases can be added as biotinylated derivatives for easy detection. A similar approach is used in SDA. Target specific sequences can also be detected using a cyclic probe reaction (CPR). In CPR, a probe having 3′ and 5′ sequences of non-specific DNA and a middle sequence of specific RNA is hybridized to DNA that is present in a sample. Upon hybridization the reaction is treated with RNase H, and the products of the probe identified as distinctive products that are released after digestion. The original template is annealed to another cycling probe and the reaction is repeated.

Still another amplification methods described in GB Application No. 2 202 328, and in PCT Application No. PCT/US89/01025, each of which is incorporated herein by reference in its entirety, may be used in accordance with the present invention. In the former application, “modified” primers are used in a PCR-like, template- and enzyme-dependent synthesis. The primers may be modified by labeling with a capture moiety (e.g., biotin) and/or a detector moiety (e.g., enzyme). In the latter application, an excess of labeled probes are added to a sample. In the presence of the target sequence, the probe binds and is cleaved catalytically. After cleavage, the target sequence is released intact to be bound by excess probe. Cleavage of the labeled probe signals the presence of the target sequence.

Other nucleic acid amplification procedures include transcription-based amplification systems (TAS), including nucleic acid sequence based amplification (NASBA) and 3SR (Kwok et al., (1989) Proc. Nat'l Acad. Sci. USA, 86: 1173; and WO 88/10315, incorporated herein by reference in their entirety). In NASBA, the nucleic acids can be prepared for amplification by standard phenol/chloroform extraction, heat denaturation of a clinical sample, treatment with lysis buffer and minispin columns for isolation of DNA and RNA or guanidinium chloride extraction of RNA. These amplification techniques involve annealing a primer which has target specific sequences. Following polymerization, DNA/RNA hybrids are digested with RNase H while double stranded DNA molecules are heat denatured again. In either case the single stranded DNA is made fully double stranded by addition of second target specific primer, followed by polymerization. The double-stranded DNA molecules are then multiply transcribed by an RNA polymerase such as T7 or SP6. In an isothermal cyclic reaction, the RNA's are reverse transcribed into single stranded DNA, which is then converted to double stranded DNA, and then transcribed once again with an RNA polymerase such as T7 or SP6. The resulting products whether truncated or complete, indicate target specific sequences.

Davey et al., EPO No. 329 822 (incorporated herein by reference in its entirety) disclose a nucleic acid amplification process involving cyclically synthesizing single-stranded RNA (“ssRNA”), ssDNA; and double-stranded DNA (dsDNA), which may be used in accordance with the present invention. The ssRNA is a template for a first primer oligonucleotide, which is elongated by reverse transcriptase (RNA-dependent DNA polymerase). The RNA is then removed from the resulting DNA:RNA duplex by the action of ribonuclease H (RNase H, an RNase specific for RNA in duplex with either DNA or RNA). The resultant ssDNA is a template for a second primer, which also includes the sequences of an RNA polymerase promoter (exemplified by T7 RNA polymerase) 5′ to its homology to the template. This primer is then extended by DNA polymerase (exemplified by the large “Klenow” fragment of E. coli DNA polymerase I), resulting in a double-stranded DNA (“dsDNA”) molecule, having a sequence identical to that of the original RNA between the primers and having additionally, at one end, a promoter sequence. This promoter sequence can be used by the appropriate RNA polymerase to make many RNA copies of the DNA. These copies can then re-enter the cycle leading to very swift amplification. With proper choice of enzymes this amplification can be done isothennally without addition of enzymes at each cycle. Because of the cyclical nature of this process, the starting sequence can be chosen to be in the form of either DNA or RNA.

PCT Application WO 89/06700 (incorporated herein by reference in its entirety) disclose a nucleic acid sequence amplification scheme based on the hybridization of a promoter/primer sequence to a target single-stranded DNA (“ssDNA”) followed by transcription of many RNA copies of the sequence. This scheme is not cyclic, i.e., new templates are not produced from the resultant RNA transcripts. Other amplification methods include “RACE” and “one-sided PCR” (Frohman, In: PCR Protocols. A Guide To Methods And Applications, Academic Press, N.Y., 1990.; and O'hara et al., (1989) Proc. Nat'l Acad. Sci. USA, 86: 5673-5677; each herein incorporated by reference in their entireties).

Methods based on ligation of two (or more) oligonucleotides in the presence of nucleic acid having the sequence of the resulting “di-oligonucleotide”, thereby amplifying the di-oligonucleotide, also may be used in the amplification step of the present, invention. (Wu et al., (1989) Genomics, 4:560, incorporated herein by reference).

Southern/Northern Blotting

Blotting techniques are well known to those of skill in the art. Southern blotting involves the use of DNA as a target, whereas Northern blotting involves the use of RNA as a target. Each provide different types of information, although cDNA blotting is analogous, in many aspects, to blotting or RNA species.

Briefly, a probe is used to target a DNA or RNA species that has been immobilized on a suitable matrix, often a filter of nitrocellulose. The different species should be spatially separated to facilitate analysis. This often is accomplished by gel electrophoresis of nucleic acid species followed by “blotting” on to the filter.

Subsequently, the blotted target is incubated with a probe (usually labeled) under conditions that promote denaturation and rehybridization. Because the probe is designed to base pair with the target, the probe will binding a portion of the target sequence under renaturing conditions. Unbound probe is then removed, and detection is accomplished as described above.

Separation Methods

It normally is desirable, at one stage or another, to separate the amplification product from the template and the excess primer for the purpose of determining whether specific amplification has occurred. In one embodiment, amplification products are separated by agarose, agarose-acrylamide or polyacrylamide gel electrophoresis using standard methods. See Sambrook et al., 1989.

Alternatively, chromatographic techniques may be employed to effect separation. There are many kinds of chromatography which may be used in the present invention: adsorption, partition, ion-exchange and molecular sieve, and many specialized techniques for using them including column, paper, thin-layer and gas chromatography (Freifelder. Physical Biochemistry Applications to Biochemistry and Molecular Biology, 2nd ed. Wm. Freeman and Co., New York, N.Y., 1982.

Detection Methods

Products may be visualized in order to confirm amplification of the marker sequences. One typical visualization method involves staining of a gel with ethidium bromide and visualization under UV light. Alternatively, if the amplification products are integrally labeled with radio- or fluorometrically-labeled nucleotides, the amplification products can then be exposed to x-ray film or visualized under the appropriate stimulating spectra, following separation.

In one embodiment, visualization is achieved indirectly. Following separation of amplification products, a labeled nucleic acid probe is brought into contact with the amplified marker sequence. The probe preferably is conjugated to a chromophore but may be radiolabeled. In another embodiment, the probe is conjugated to a binding partner, such as an antibody or biotin, and the other member of the binding pair carries a detectable moiety.

In one embodiment, detection is by a labeled probe. The techniques involved are well known to those of skill in the art and can be found in many standard books on molecular protocols. See Sambrook et al., 1989. For example, chromophore or radiolabel probes or primers identify the target during or following amplification.

One example of the foregoing is described in U.S. Pat. No. 5,279,721, incorporated herein by reference, which discloses an apparatus and method for the automated electrophoresis and transfer of nucleic acids. The apparatus permits electrophoresis and blotting without external manipulation of the gel and is ideally suited to carrying out methods according to the present invention.

In addition, the amplification products described above may be subjected to sequence analysis to identify specific kinds of variations using standard sequence analysis techniques. Within certain methods, exhaustive analysis of genes is carried out by sequence analysis using primer sets designed for optimal sequencing (Pignon et al, (1994) Hum. Mutat., 3:126-132, 1994). The present invention provides methods by which any or all of these types of analyses may be used. Using the sequences disclosed herein, oligonucleotide primers may be designed to permit the amplification of sequences throughout the GHR gene that may then be analyzed by direct sequencing.

Any of a variety of sequencing reactions known in the art can be used to directly sequence the GHR gene by comparing the sequence of the sample with the corresponding wild-type (control) sequence. Examples of sequencing reactions include those based on techniques developed by Maxam and Gilbert ((1977) Proc. Natl. Acad. Sci. USA 74:560) or Sanger ((1977) Proc. Natl. Acad. Sci. USA 74:5463). It is also contemplated that any of a variety of automated sequencing procedures can be utilized when performing the diagnostic assays.

Kit Components

All the essential materials and reagents required for detecting and sequencing GHR and variants thereof may be assembled together in a kit. This generally will comprise preselected primers and probes. Also included may be enzymes suitable for amplifying nucleic acids including various polyrnerases (RT, Taq, Sequenase™ etc.), deoxynucleotides and buffers to provide the necessary reaction mixture for amplification. Such kits also generally will comprise, in suitable means, distinct containers for each individual reagent and enzyme as well as for each primer or probe.

Design and Titeoretical Considerations for Relative Quantitative RT-PCR™.

Reverse transcription (RT) of RNA to cDNA followed by relative quantitative PCR (RT-PCR) can be used to determine the relative concentrations of specific mRNA species isolated from subjects. By determining that the concentration of a specific MRNA species varies, it is shown that the gene encoding the specific MRNA species is differentially expressed. Quantitative PCR may be useful for example in examining relative levels of GHRd3 and GHRfl mRNA in subjects to be treated with an agent acting via the GHR pathway, in a subject suspected of suffering from diminished GHR activity, or preferably suffering from short stature, obesity, infection, or diabetes; acromegaly or gigantism conditions which could be associated with lactogenic, diabetogenic, lipolytic and protein anabolic effects; conditions associated with sodium and water retention; metabolic syndromes; mood and sleep disorders, cancer, cardiac disease or hypertension.

In PCR, the number of molecules of the amplified target DNA increase by a factor approaching two with every cycle of the reaction until some reagent becomes limiting. Thereafter, the rate of amplification becomes increasingly diminished until there is no increase in the amplified target between cycles. If a graph is plotted in which the cycle number is on the X axis and the log of the concentration of the amplified target DNA is on the Y axis, a curved line of characteristic shape is formed by connecting the plotted points. Beginning with the first cycle, the slope of the line is positive and constant. This is said to be the linear portion of the curve. After a reagent becomes limiting, the slope of the line begins to decrease and eventually becomes zero. At this point the concentration of the amplified target DNA becomes asymptotic to some fixed value. This is said to be the plateau portion of the curve.

The concentration of the target DNA in the linear portion of the PCR amplification is directly proportional to the starting concentration of the target before the reaction began. By determining the concentration of the amplified products of the target DNA in PCR reactions that have completed the same number of cycles and are in their linear ranges, it is possible to determine the relative concentrations of the specific target sequence in the original DNA mixture. If the DNA mixtures are cDNAs synthesized from RNAs isolated from different tissues or cells, the relative abundances of the specific mRNA from which the target sequence was derived can be determined for the respective tissues or cells. This direct proportionality between the concentration of the PCR products and the relative mRNA abundances is only true in the linear range of the PCR reaction.

The final concentration of the target DNA in the plateau portion of the curve is determined by the availability of reagents in the reaction mix and is independent of the original concentration of target DNA. Therefore, the first condition that must be met before the relative abundances of a mRNA species can be determined by RT-PCR for a collection of RNA populations is that the concentrations of the amplified PCR products must be sampled when the PCR reactions are in the linear portion of their curves.

The second condition that must be met for an RT-PCR experiment to successfully determine the relative abundances of a particular mRNA species is that relative concentrations of the amplifiable cDNAs must be normalized to some independent standard. The goal of an RT-PCR experiment is to determine the abundance of a particular mRNA species relative to the average abundance of all MRNA species in the sample. In the experiments described below, mRNAs for GHRfl can be used as standards to which the relative abundance of GHRd3 mRNAs are compared.

Most protocols for competitive PCR utilize internal PCR standards that are approximately as abundant as the target. These strategies are effective if the products of the PCR amplifications are sampled during their linear phases. If the products are sampled when the reactions are approaching the plateau phase, then the less abundant product becomes relatively over represented. Comparisons of relative abundances made for many different RNA samples, such as is the case when examining RNA samples for differential expression, become distorted in such a way as to make differences in relative abundances of RNAs appear less than they actually are. This is not a significant problem if the internal standard is much more abundant than the target. If the internal standard is more abundant than the target, then direct linear comparisons can be made between RNA samples.

The above discussion describes theoretical considerations for an RT-PCR assay for clinically derived materials. The problems inherent in clinical samples are that they are of variable quantity (making normalization problematic), and that they are of variable quality (necessitating the co-amplification of a reliable internal control, preferably of larger size than the target). Both of these problems are overcome if the RT-PCR is performed as a relative quantitative RT-PCR with an internal standard in which the internal standard is an amplifiable cDNA fragment that is larger than the target cDNA fragment and in which the abundance of the mRNA encoding the internal standard is roughly 5-100 fold higher than the mRNA encoding the target. This assay measures relative abundance, not absolute abundance of the respective MRNA species.

Other studies may be performed using a more conventional relative quantitative RT-PCR assay with an external standard protocol. These assays sample the PCR products in the linear portion of their amplification curves. The number of PCR cycles that are optimal for sampling must be empirically determined for each target cDNA fragment. In addition, the reverse transcriptase products of each RNA population isolated from the various tissue samples must be carefully normalized for equal concentrations of amplifiable cDNAs. This consideration is very important since the assay measures absolute mRNA abundance. Absolute mRNA abundance can be used as a measure of differential gene expression only in normalized samples. While empirical determination of the linear range of the amplification curve and normalization of cDNA preparations are tedious and time consuming processes, the resulting RT-PCR assays can be superior to those derived from the relative quantitative. RT-PCR assay with an internal standard.

One reason for this advantage is that without the internal standard/competitor, all of the reagents can be converted into a single PCR product in the linear range of the amplification curve, thus increasing the sensitivity of the assay. Another reason is that with only one PCR product, display of the product on an electrophoretic gel or another display method becomes less complex, has less background and is easier to interpret.

Chip Technologies

Specifically contemplated by the present inventors are chip-based DNA technologies such as those described by Hacia et al., ((1996) Nature Genetics, 14:441447) and Shoemaker et al., ((1996) Nature Genetics 14:450456. Briefly, these techniques involve quantitative methods for analyzing large numbers of genes rapidly and accurately. By tagging genes with oligonucleotides or using fixed probe arrays, one can employ chip technology to segregate target molecules as high density arrays and screen these molecules on the basis of hybridization. See also Pease et al, ((1994) Proc. Nat'l Acad Sci. USA, 91:5022-5026); Fodor et al., ((1991) Science, 251:767-773).

Methods of Detecting GHRd3 or GHRfl Protein

Antibodies can be used in characterizing the GHRd3 and/or GHRfl content of healthy and diseased tissues, through techniques such as ELISAs and Western blotting. Methods for obtaining GHRd3 and GHRfl polypeptides are further described herein and can be carried out using known methods. Likewise, methods of preparing antibodies capable of selectively binding GHRd3 and GHRfl isoforms are further described herein.

In one example, GHR antibodies, including GHRd3, GHRfl and GHR antibodies that do not distinguish between GHRd3 and GHRfl, can be used in an ELISA assay is contemplated. For example, anti-GHR antibodies are immobilized onto a selected surface, preferably a surface exhibiting a protein affinity such as the wells of a polystyrene microtiter plate. After washing to remove incompletely adsorbed material, it is desirable to bind or coat the assay plate wells with a non-specific protein that is known to be antigenically neutral with regard to the test antisera such as bovine serum albumin (BSA), casein or solutions of powdered milk. This allows for blocking of non-specific adsorption sites on the immobilizing surface and thus reduces the background caused by non-specific binding of antigen onto the surface.

After binding of antibody to the well, coating with a non-reactive material to reduce background, and washing to remove unbound material, the immobilizing surface is contacted with the sample to be tested in a manner conducive to immune complex (antigen/antibody) formation.

Following formation of specific immunocomplexes between the test sample and the bound antibody, and subsequent washing, the occurrence and even amount of immunocomplex formation may be determined by subjecting same to a second antibody having specificity for GHR that differs the first antibody. Appropriate conditions preferably include diluting the sample with diluents such as BSA, bovine gamma globulin (BGG) and phosphate buffered saline (PBS)/Tween. These added agents also tend to assist in the reduction of nonspecific background. The layered antisera is then allowed to incubate for from about 2 to about 4 hr, at temperatures preferably on the order of about 25 C to about 27 C. Following incubation, the antisera-contacted surface is washed so as to remove non-immunocomplexed material. A preferred washing procedure includes washing with a solution such as PBS/Tween or borate buffer.

To provide a detecting means, the second antibody will preferably have an associated enzyme that will generate a color development upon incubating with an appropriate chromogenic substrate. Thus, for example, one will desire to contact and incubate the second antibody-bound surface with a urease or peroxidase-conjugated anti-human IgG for a period of time and under conditions which favor the development of immunocomplex formation (e.g., incubation for 2 hr at room temperature in a PBS-containing solution such as PBS/Tween).

After incubation with the second enzyme-tagged antibody, and subsequent to washing to remove unbound material, the amount of label is quantified by incubation with a chromogenic substrate such as urea and bromocresol purple or 2,2′-azino-di-(3-ethyl-benzthiazoline)-6-sulfonic acid (ABTS) and H₂O₂, in the case of peroxidase as the enzyme label. Quantitation is then achieved by measuring the degree of color generation, e.g., using a visible spectrum spectrophotometer.

The preceding format may be altered by first binding the sample to the assay plate. Then, primary antibody is incubated with the assay plate, followed by detecting of bound primary antibody using a labeled second antibody with specificity for the primary antibody.

The steps of various other useful immunodetection methods have been described in the scientific literature, such as, eg., Nakamura et al., In: Handbook of Experimental Immunology (4th Ed.), Weir. E., Herzenberg, L. A. Blackwell, C., Herzenberg, L. (eds). Vol. 1. Chapter 27, Blackwell Scientific Publ., Oxford, 1987; incorporated herein by reference). Immunoassays, in their most simple and direct sense, are binding assays. Certain preferred immunoassays are the various types of radioimmunoassays (RIA) and immunobead capture assay. Immunohistochemical detection using tissue-sections also is particularly useful. However, it will be readily appreciated that detection is not limited to such techniques, and Western blotting, dot blotting, FACS analyses, and the like also may be used in connection with the present invention.

In a preferred example, GHRd3 levels can be detected using a GHRd3-specific antibody using the methods described above. In other methods, the total amount of GHR is determined without differentiating between GHRd3 and GHRfl, and the amount of GHRfl is determined. The difference in amount of undifferentiated GHR and GHRfl indicates the amount of GHRd3 present.

Preferably such methods detect GHBP (e.g. the extracellular portion of GHRd3 or GHRfl) in circulation. Preferred examples of procedures allow detection of undifferentiated GHR (e.g. for deducing GHRd3 from total undifferentiated GHR compared to GHRfl), detection of GHRd3 and/or detection of GHRfl. Such procedures include the ELISA assay, the ligand-mediated immunofunctional assay (LIFA) and the radioimmunoassay (RIA).

LIFA for the detection of undifferentiated (e.g. GHRd3 or GHRfl) GHR can be carried out according to the methods of Pflaum et al. ((1993) Exp. Clin. Endocrinol. 101 (Suppl. 1): 44) and Kratzsch et al. ((2001) Clin. Endocrinol. 54: 61-68. Briefly, in one example, undifferentiated GHR is detected using a monoclonal anti rGHBP antibody for coating microtiter plates. Serum sample or glycosylated rGHBP standards are incubated together with 10 ng/well hGH and a monoclonal antibody directed against hGH as biotinylated tracer. The signal is amplified by the europium-labeled streptavidin system and measured using a fluorometer. In another example, a competitive radioimmunoassay (RIA) is carried out to detect undifferentiated GHBP, using an anti-rhGHBP antibody, rhGHBP standards and 1251-rhGHBP as labeled antigen as described in Kratsch et al. ((1995) Eur. J. Endocrinol. 132: 306-312).

In another example described in Kratzsch et al. ((2001) Clin. Endocrinol. 54: 61-68), undifferentiated GHBP is detected by coating a microtiter plate is coated with 100 μl of the monoclonal antibody 10B8 which binds GHBP outside of the hGH binding site (Rowlinson et al. (1999), in 50 mmol//l sodium phosphate buffer, pH 9.6 After a washing step, 25 μl sample or standard and 50 ng biotin-labeled anti-GHGBP mAb 5C6 (which binds GHBP within the hGH binding site (Rowlinson et al (1999)) in 75 μl assay buffer (50 mM Tris-(hydroxymethyl)-aminomethane, 150, mM NaCl, 0.05% NaN3, 0.01% Tween 40, 0.5% BSA 0.05% bovine gamma-globulin, 20 μmol/l diethylenetriaminepenta acetic acid) are added and incubated overnight. The amount of GHRfl is then determined using an antibody specific for the exon 3-containing fl form of GHBP). Briefly, mAb 10B8 is immobilized on microtiter plates as in the case of undifferentiated GHBP. After a washing step, 25 μl sample or standard and 75 μl of a rabbit polyclonal antibody against GHRd3 peptide described in Kratzsch et al. (2001) (diluted 1:10000) are added and incubated overnight. 20 ng biotinylated murine antirabbit IgG is added to each well and incubated for 2 h followed by repeated rinsing. The signals are amplified by the europium-labeled streptavidin system and measured using a fluorometer. Recombinant nonglycosylated hGHBP, diluted in sheep serum, is used as a standard.

Antibodies specific for GHRd3 for use according to the present invention can be obtained using known methods. An isolated GHRd3 protein, or a portion or fragment thereof, can be used as an immunogen to generate antibodies that bind GHRd3 using standard techniques for polyclonal and monoclonal antibody preparation. A GHRd3 protein can be used or, alternatively, the invention provides antigenic peptide fragments of GHRd3 for use as immunogens.

GHRd3 polypeptides can be prepared using known means, either by purification from a biological sample obtained from an individual or more preferably as recombinant polypeptides. The GHRfl amino acid sequence is shown in SEQ ID NOS: 2 and 3, from which GHRd3 differs by a deletion of 22 amino acids encoded by exon 3. The antigenic peptide of GHRd3 preferably comprises at least 8 amino acid residues of the amino acid sequence shown in SEQ ID NOS: 2 and 3, wherein at least one amino acid is outside of said exon 3-encoded amino acid residues. Said antigenic peptide encompasses an epitope of GHRd3 such that an antibody raised against the peptide forms a specific immune complex with GHRd3. Preferably the antibody binds selectively or preferentially to GHRd3 and does not substantially bind to GHRfl. Preferably, the antigenic peptide comprises at least 10 amino acid residues, more preferably at least 15 amino acid residues, even more preferably at least 20 amino acid residues, and most preferably at least 30 amino acid residues.

Preferred epitopes encompassed by the antigenic peptide are regions of GHRd3 that are located on the surface of the protein, e.g., hydrophilic regions.

A GHRd3 immunogen typically is used to prepare antibodies by immunizing a suitable subject, (e.g., rabbit, goat, mouse or other mammal) with the immunogen. An appropriate immunogenic preparation can contain, for example, recombinantly expressed GHRd3 protein or a chemically synthesized GHRd3 polypeptide. The preparation can further include an adjuvant, such as Freund's complete or incomplete adjuvant, or similar immunostimulatory agent. Immunization of a suitable subject with an immunogenic GHRd3 preparation induces a polyclonal anti-GHRd3 antibody response.

Accordingly, another aspect of the invention pertains to anti-GHRd3 antibodies. The term “antibody” as used herein refers to immunoglobulin molecules and immunologically active portions of immunoglobulin molecules, i.e., molecules that contain an antigen binding site which specifically binds (imrnunoreacts with) an antigen, such as GHRd3. Examples of immunologically active portions of immunoglobulin molecules include F(ab) and F(ab′)₂ fragments which can be generated by treating the antibody with an enzyme such as pepsin. The invention provides polyclonal and monoclonal antibodies that bind GHRd3. The term “monoclonal antibody” or “monoclonal antibody composition”, as used herein, refers to a population of antibody molecules that contain only one species of an antigen binding site capable of immunoreacting with a particular epitope of GHRd3. A monoclonal antibody composition thus typically displays a single binding affinity for a particular GHRd3 protein with which it immunoreacts.

The invention concerns antibody compositions, either polyclonal or monoclonal, capable of selectively binding, or selectively bind to an epitope-containing a polypeptide comprising a contiguous span of at least 6 amino acids, preferably at least 8 to 10 amino acids, more preferably at least 12, 15, 20, 25, 30, 40, 50, or 100 amino acids of SEQ ID NO: 2 or 3, said contiguous span preferably including at least one amino acid outside of said 22 amino acid span encoded by exon 3 of the GHR gene.

Polyclonal anti-GHRd3 antibodies can be prepared as described above by immunizing a suitable subject with a GHRd3 immunogen. The anti-GHRd3 antibody titer in the immunized subject can be monitored over time by standard techniques, such as with an enzyme linked immunosorbent assay (ELISA) using immobilized GHRd3. If desired, the antibody molecules directed against GHRd3 can be isolated from the mammal (e.g., from the blood) and further purified by well known techniques, such as protein A chromatography to obtain the IgG fraction. At an appropriate time after immunization, e.g., when the anti-GHRd3 antibody titers are highest, antibody-producing cells can be obtained from the subject and used to prepare monoclonal antibodies by standard techniques, such as the hybridoma technique originally described by Kohler and Milstein (1975) Nature 256:495497) (see also, Brown et al. (1981) J. Immunol. 127:53946; Brown et al. (1980) J. Biol. Chem. 255:4980-83; Yeh et al. (1976) PNAS 76:2927-31; and Yeh et al. (1982) Int. J. Cancer 29:269-75), the more recent human B cell hybridoma technique (Kozbor et al. (1983) Immunol Today 4:72), the EBV-hybridoma technique (Cole et al. (1985), Monoclonal Antibodies and Cancer Therapy, Alan R. Liss, Inc., pp. 77-96) or trioma techniques. The technology for producing monoclonal antibody hybridomas is well known (see generally R. H. Kenneth, in Monoclonal Antibodies: A New Dimension In Biological Analyses, Plenum Publishing Corp., New York, N.Y. (1980); E. A. Lemer (1981) Yale J. Biol. Med., 54:387402; M. L. Gefter et al. (1977) Somatic Cell Genet. 3:231-36). Briefly, an immortal cell line (typically a myeloma) is fused to lymphocytes (typically splenocytes) from a mammal immunized with a GHRd3 immunogen as described above, and the culture supernatants of the resulting hybridoma cells are screened to identify a hybridoma producing a monoclonal antibody that binds GHRd3.

Any of the many well known protocols used for fusing lymphocytes and immortalized cell lines can be applied for the purpose of generating an anti-GHRd3 monoclonal antibody (see, e.g., G. Galfre et al. (1977) Nature 266:55052; Gefter et al. Somatic Cell Genet., cited supra; Lemer, Yale J Biol. Med, cited supra; Kenneth, Monoclonal Antibodies, cited supra). Moreover, the ordinarily skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O-Ag14 myeloma lines. These myeloma lines are available from ATCC. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind GHRd3, e.g., using a standard ELISA assay.

Alternative to preparing monoclonal antibody-secreting hybridomas, a monoclonal anti-GHRd3 antibody can be identified and isolated by screening a recombinant combinatorial immunoglobulin library (e.g., an antibody phage display library) with GHRd3 to thereby isolate immunoglobulin library members that bind GHRd3. Kits for generating and screening phage display libraries are commercially available (e.g., the Pharmacia Recombinant Phage Antibody System, Catalog No. 27-9400-01; and the Stratagene SurfZAP.TM. Phage Display Kit, Catalog No. 240612). Additionally, examples of methods and reagents particularly amenable for use in generating and screening antibody display library can be found in, for example, Ladner et al. U.S. Pat. No. 5,223,409; Kang et al. PCT International Publication No. WO 92/18619; Dower et al. PCT International Publication No. WO 91/17271; Winter et al. PCT International Publication WO 92/20791; Markland et al. PCT International Publication No. WO 92/15679; Breitling et al. PCT International Publication WO 93/01288; McCafferty et al. PCT International Publication No. WO 92/01047; Garrard et al. PCT International Publication No. WO 92/09690; Ladner et al. PCT International Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J 12:725-734; Hawkins et al. (1992) J. Mol. Biol. 226:889-896; Clarkson et al. (1991) Nature 352:624-628; Gram et al. (1992) PNAS 89:3576-3580; Garrad et al. (1991) Bio/Technology 9:1373-1377; Hoogenboom et al. (1991) Nuc. Acid Res. 19:41334137; Barbas et al. (1991) PNAS 88:7978-7982; and McCafferty et al. Nature (1990) 348:552-554.

An anti-GHRd3 antibody (e.g., monoclonal antibody) can be used to isolate GHRd3 by standard techniques, such as affinity chromatography or immunoprecipitation. An anti-GHRd3 antibody can facilitate the purification of natural GHRd3 from cells and of recombinantly produced GHRd3 expressed in host cells. Moreover, an anti-GHRd3 antibody can be used to detect GHRd3 protein (e.g., in a cellular lysate or cell supernatant) in order to evaluate the abundance and pattern of expression of the GHRd3 protein. Anti-GHRd3 antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, -galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include 125I, ¹³¹I, ³⁵S or ³H.

In a preferred example, substantially pure GHRd3 protein or polypeptide is obtained. The concentration of protein in the final preparation is adjusted, for example, by concentration on an Amicon filter device, to the level of a few micrograms per ml. Monoclonal or polyclonal antibodies to the protein can then be prepared as follows: Monoclonal Antibody Production by Hybridoma Fusion Monoclonal antibody to epitopes in the GHRd3 or a portion thereof can be prepared from murine hybridomas according to the classical method of Kohler and Milstein (Nature, 256: 495, 1975) or derivative methods thereof (see Harlow and Lane, Antibodies A Laboratory Manual, Cold Spring Harbor Laboratory, pp. 53-242, 1988).

Briefly, a mouse is repetitively inoculated with a few micrograms of the GHRd3 or a portion thereof over a period of a few weeks. The mouse is then sacrificed, and the antibody producing cells of the spleen isolated. The spleen cells are fused by means of polyethylene glycol with mouse myeloma cells, and the excess unfused cells destroyed by growth of the system on selective media comprising aminopterin (HAT media). The successfully fused cells are diluted and aliquots of the dilution placed in wells of a microtiter plate where growth of the culture is continued. Antibody-producing clones are identified by detection of antibody in the supernatant fluid of the wells by immunoassay procedures, such as ELISA, as original described by Engvall, E., Meth. Enzymol. 70: 419 (1980). Selected positive clones can be expanded and their monoclonal antibody product harvested for use. Detailed procedures for monoclonal antibody production are described in Davis, L. et al. Basic Methods in Molecular Biology Elsevier, New York. Section 21-2.

The antibody compositions of the present invention will find great use in immunoblot or Western blot analysis. The antibodies may be used as high-affinity primary reagents for the identification of proteins immobilized onto a solid support matrix, such as nitrocellulose, nylon or combinations thereof. In conjunction with immunoprecipitation, followed by gel electrophoresis, these may be used as a single step reagent for use in detecting antigens against which secondary reagents used in the detection of the antigen cause an adverse background. Immunologically-based detection methods for use in conjunction with Western blotting include enzymatically-, radiolabel-, or fluorescently-tagged secondary antibodies against the toxin moiety are considered to be of particular use in this regard. U.S. Patents concerning the use of such labels include U.S. Pat. Nos. 3,817,837; 3,850,752; 3,939,350; 3,996,345; 4,277,437; 4,275,149 and 4,366,241, each incorporated herein by reference. Of course, one may find additional advantages through the use of a secondary binding ligand such as a second antibody or a biotin/avidin ligand binding arrangement, as is known in the art.

### Administration of GH Compositions

The GH to be used in accordance with the invention may be in native-sequence or in variant form, and from any source, whether natural, synthetic, or recombinant. Examples include human growth hormone (hGH), which is natural or recombinant GH with the human native sequence (GENOTROPIN™, somatotropin or somatropin), and recombinant growth hormone (rGH), which refers to any GH or GH variant produced by means of recombinant DNA technology, including somatrem, somatotropin, and somatropin. Preferred herein for human use is recombinant human native-sequence, mature GH with or without a methionine at its N-terminus. Most preferred is GENOTROP™ (Pharmacia, U.S.A.) which is a recombinant human GH polypeptide. Also preferred is methionyl human growth hormone (met-hGH) produced in E. coli, e.g., by the process described in U.S. Pat. No. 4,755,465 issued Jul. 5, 1988 and Goeddel et al., Nature, 282: 544 (1979). Met-hGH, sold as PROTROPIN™ (Genentech, Inc. U.S.A.), is identical to the natural polypeptide, with the exception of the presence of an N-terminal methionine residue. Another example is recombinant hGH sold as NUTROPIN™ (Genentech, Inc., U.S.A.). This latter hGH lacks this methionine residue and has an amino acid sequence identical to that of the natural hormone. See Gray et al., Biotechnology 2: 161 (1984). Another GH example is an hGH variant that is a placental form of GH with pure somatogenic and no lactogenic activity as described in U.S. Pat. No. 4,670,393. Also included are GH variants, for example such as those described in WO 90/04788 and WO 92/09690.

Other examples include GH compositions that act as GHR antagonists, such as pegvisomant (SOMAVERT™, Pharmacia, U.S.A.) which can be used for the treatment of acromegaly. GH can be directly administered to a subject by any suitable technique, including parenterally, intranasally, intrapulmonary, orally, or by absorption through the skin. They can be administered locally or systemically. Examples of parenteral administration include subcutaneous, intramuscular, intravenous, intraarterial, and intraperitoneal administration. Preferably, they are administered by daily subcutaneous injection.

The GH to be used in the therapy will be formulated and dosed in a fashion consistent with good medical practice, taking into account the clinical condition of the individual subject (especially the side effects of treatment with GH alone), the site of delivery of the GH composition(s), the method of administration, the scheduling of administration, and other factors known to practitioners. The “effective amounts” of each component for purposes herein are thus determined by such considerations and are amounts that increase the growth rates of the subjects.

For GH, a dose of greater than about 0.2 mg/kg/week is preferably employed, more preferably greater than about 0.25 mg/kg/week, and even more preferably greater than or equal to about 0.3 mg/kg/week. In one embodiment, the dose of GH ranges from about 0.3 to 1.0 mg/kg/week, and in another embodiment, 0.35 to 1.0 mg/kg/week.

Preferably, the GH is administered once per day subcutaneously. In preferred aspects, the dose of GH is between about 0.001 and 0.2 mg/kg/day. Yet more preferably, the dose of GH is between about 0.010 and 0.10 mg/kg/day.

As discussed, subjects homozygous or heterozygous for the GHRd3 allele are expected to have a greater positive response to GH treatment than subjects homozygous for the GHRfl allele. In preferred aspects, a dose administered to subjects homozygous for the GHRfl allele will be greater than the dose administered to a subject that is homozygous or heterozygous for the GHRd3 allele.

The GH is suitably administered continuously or non-continuously, such as at particular times (e.g., once daily) in the form of an injection of a particular dose, where there will be a rise in plasma GH concentration at the time of the injection, and then a drop in plasma GH concentration until the time of the next injection. Another non-continuous administration method results from the use of PLGA microspheres and many implant devices available that provide a discontinuous release of active ingredient, such as an initial burst, and then a lag before release of the active ingredient. See, e.g., U.S. Pat. No. 4,767,628.

The GH may also be administered so as to have a continual presence in the blood that is maintained for the duration of the administration of the GH. This is most preferably accomplished by means of continuous infusion via, e.g., mini-pump such as an osmotic mini-pump. Alternatively, it is properly accomplished by use of frequent injections of GH (i.e., more than once daily, for example, twice or three times daily).

In yet another embodiment, GH may be administered using long-acting GH formulations that either delay the clearance of GH from the blood or cause a slow release of GH from, e.g., an injection site. The long-acting formulation that prolongs GH plasma clearance may be in the form of GH complexed, or covalently conjugated (by reversible or irreversible bonding) to a macromolecule such as one or more of its binding proteins (WO 92/08985) or a water-soluble polymer selected from PEG and polypropylene glycol homopolymers and polyoxyethylene polyols, i.e., those that are soluble in water at room temperature. Alternatively, the GH may be complexed or bound to a polymer to increase its circulatory half-life. Examples of polyethylene polyols and polyoxyethylene polyols useful for this purpose include polyoxyethylene glycerol, polyethylene glycol, polyoxyethylene sorbitol, polyoxyethylene glucose, or the like. The glycerol backbone of polyoxyethylene glycerol is the same backbone occurring in, for example, animals and humans in mono-, di-, and triglycerides.

The polymer need not have any particular molecular weight, but it is preferred that the molecular weight be between about 3500 and 100,000, more preferably between 5000 and 40,000. Preferably the PEG homopolymer is unsubstituted, but it may also be substituted at one end with an alkyl group. Preferably, the alkyl group is a C1-C4 alkyl group, and most preferably a methyl group. Most preferably, the polymer is an unsubstituted homopolymer of PEG, a monomethyl-substituted homopolymer of PEG (mPEG), or polyoxyethylene glycerol (POG) and has a molecular weight of about 5000 to 40,000.

The GH is covalently bonded via one or more of the amino acid residues of the GH to a terminal reactive group on the polymer, depending mainly on the reaction conditions, the molecular weight of the polymer, etc. The polymer with the reactive group(s) is designated herein as activated polymer. The reactive group selectively reacts with free amino or other reactive groups on the GH. It will be understood, however, that the type and amount of the reactive group chosen, as well as the type of polymer employed, to obtain optimum results, will depend on the particular GH employed to avoid having the reactive group react with too many particularly active groups on the GH. As this may not be possible to avoid completely, it is recommended that generally from about 0.1 to 1000 moles, preferably 2 to 200 moles, of activated polymer per mole of protein, depending on protein concentration, is employed. The final amount of activated polymer per mole of protein is a balance to maintain optimum activity, while at the same time optimizing, if possible, the circulatory half-life of the protein.

While the residues may be any reactive amino acids on the protein, such as one or two cysteines or the N-terminal amino acid group, preferably the reactive amino acid is lysine, which is linked to the reactive group of the activated polymer through its free epsilon-amino group, or glutamic or aspartic acid, which is linked to the polymer through an amide bond.

The covalent modification reaction may take place by any appropriate method generally used for reacting biologically active materials with inert polymers, preferably at about pH 5-9, more preferably 7-9 if the reactive groups on the GH are lysine groups. Generally, the process involves preparing an activated polymer (with at least one terminal hydroxyl group), preparing an active substrate from this polymer, and thereafter reacting the GH with the active substrate to produce the GH suitable for formulation. The above modification reaction can be performed by several methods, which may involve one or more steps. Examples of modifying agents that can be used to produce the activated polymer in a one-step reaction include cyanuric acid chloride (2,4,6-trichloro-S-triazine) and cyanuric acid fluoride.

In one embodiment the modification reaction takes place in two steps wherein the polymer is reacted first with an acid anhydride such as succinic or glutaric anhydride to form a carboxylic acid, and the carboxylic acid is then reacted with a compound capable of reacting with the carboxylic acid to form an activated polymer with a reactive ester group that is capable of reacting with the GH. Examples of such compounds include N-hydroxysuccinimide, 4-hydroxy-3-nitrobenzene sulfonic acid, and the like, and preferably N-hydroxysuccinimide or 4-hydroxy-3-nitrobenzene sulfonic acid is used. For example, monomethyl substituted PEG may be reacted at elevated temperatures, preferably about 100-110 C for four hours, with glutaric anhydride. The monomethyl PEG-glutaric acid thus produced is then reacted with N-hydroxysuccinimide in the presence of a carbodiimide reagent such as dicyclohexyl or isopropyl carbodiimide to produce the activated polymer, methoxypolyethylene glycolyl-N-succinimidyl glutarate, which can then be reacted with the GH. This method is described in detail in Abuchowski et al., Cancer Biochem. Biophys., 7: 175-186 (1984). In another example, the monomethyl substituted PEG may be reacted with glutaric anhydride followed by reaction with 4-hydroxy-3-nitrobenzene sulfonic acid (HNSA) in the presence of dicyclohexyl carbodiimide to produce the activated polymer. HNSA is described by Bhatnagar et al., Peptides: Synthesis-Structure-Function, Proceedings of the Seventh American Peptide Symposium, Rich et al. (eds.) (Pierce Chemical Co., Rockford, Ill., 1981), p. 97-100, and in Nitecki et al., High-Technology Route to Virus Vaccines (American Society for Microbiology: 1986) entitled “Novel Agent for Coupling Synthetic Peptides to Carriers and Its Applications.”

Specific methods of producing GH conjugated to PEG include the methods described in U.S. Pat. No. 4,179,337 on PEG-GH and U.S. Pat. No. 4,935,465, which discloses PEG reversibly but covalently linked to GH.

The GH can also be suitably administered by sustained-release systems. Examples of sustained-release compositions useful herein include semi-permeable polymer matrices in the form of shaped articles, e.g., films, or microcapsules. Sustained-release matrices include polylactides (U.S. Pat. No. 3,773,919, EP 58,481), copolymers of L-glutamic acid and gamma-ethyl-L-glutamate (Sidman et al., Biopolymers, 22, 547-556 (1983), poly(2-hydroxyethyl methacrylate) (Langer et al., J. Biomed. Mater. Res., 15: 167-277 (1981); Langer, Chem. Tech., 12: 98-105 (1982), ethylene vinyl acetate (Langer et al., supra) or poly-D-(−)-3-hydroxybutyric acid (EP 133,988), or PLGA microspheres.

Sustained-release GH compositions also include liposomally entrapped GH. Liposomes containing GH are prepared by methods known per se: DE 3,218,121; Epstein et al., Proc. Natl. Acad. Sci. USA, 82: 3688-3692 (1985); Hwang et al., Proc. Natl. Acad. Sci. USA, 77: 40304034 (1980); EP 52,322; EP 36,676; EP 88,046; EP 143,949; EP 142,641; Japanese Pat. Appln. 83-118008; U.S. Pat. Nos. 4,485,045 and 4,544,545; and EP 102,324. ordinarily, the liposomes are of the small (about 200-800 Angstroms) unilamellar type in which the lipid content is greater than about 30 mol. percent cholesterol, the selected proportion being adjusted for the optimal therapy. In addition, a biologically active sustained-release formulation can be made from an adduct of the GH covalently bonded to an activated polysaccharide as described in U.S. Pat. No. 4,857,505. In addition, U.S. Pat. No. 4,837,381 describes a microsphere composition of fat or wax or a mixture thereof and GH for slow release.

In another embodiment, the subjects identified above are also treated with an effective amount of IGF-I. As a general proposition, the total pharmaceutically effective amount of IGF-I administered parenterally per dose will be in the range of about 50 to 240μg/kg/day, preferably 100 to 200 μg/kg/day, of subject body weight, although, as noted above, this will be subject to a great deal of therapeutic discretion. Also, preferably the IGF-I is administered once or twice per day by subcutaneous injection. In a further embodiment, both IGF-I and GH can be administered to the subject, each in effective amounts, or each in amounts that are sub-optimal but when combined are effective. Preferably about 0.001 to 0.2 mg/kg/day or more preferably about 0.01 to 0.1 mg/kg/day GH is administered. Preferably, the administration of both IGF-I and GH is by injection using, e.g., intravenous or subcutaneous means. More preferably, the administration is by subcutaneous injection for both IGF-I and GH, most preferably daily injections.

It is noted that practitioners devising doses of both IGF-I and GH should take into account the known side effects of treatment with these hormones. For GH, the side effects include sodium retention and expansion of extracellular volume (Ikkos et al., Acta Endocrinol. (Copenhagen), 32: 341-361 (1959); Biglieri et al., J. Clin. Endocrinol. Metab., 21: 361-370 (1961), as well as hyperinsulinemia and hyperglycemia. The major apparent side effect of IGF-I is hypoglycemia. Guler et al., Proc. Natl. Acad. Sci. USA, 86: 2868-2872 (1989). Indeed, the combination of IGF-I and GH may lead to a reduction in the unwanted side effects of both agents (e.g., hypoglycemia for IGF-I and hyperinsulinism for GH) and to a restoration of blood levels of GH, the secretion of which is suppressed by IGF-I.

For parenteral administration, in one embodiment, GH is formulated generally by mixing the GH at te desired degree of purity, in a unit dosage injectable form (solution, suspension, or emulsion), with a pharmaceutically acceptable carrier, i.e., one that is non-toxic to recipients at the dosages and concentrations employed and is compatible with other ingredients of the formulation. For example, the formulation preferably does not include oxidizing agents and other compounds that are known to be deleterious to polypeptides. Generally, the formulations are prepared by contacting the GH with liquid carriers or finely divided solid carriers or both. Then, if necessary, the product is shaped into the desired formulation. Preferably the carrier is a parenteral carrier, more preferably a solution that is isotonic with the blood of the recipient. Examples of such carrier vehicles include water, saline, Ringer's solution, and dextrose solution. Non-aqueous vehicles such as fixed oils and ethyl oleate are also useful herein, as well as liposomes.

The carrier suitably contains minor amounts of additives such as substances that enhance isotonicity and chemical stability. Such materials are non-toxic to recipients at the dosages and concentrations employed, and include buffers such as phosphate, citrate, succinate, acetic acid, and other organic acids or their salts; antioxidants such as ascorbic acid; low molecular weight (less than about ten residues) polypeptides, e.g., polyarginine or tripeptides; proteins, such as serum albumin, gelatin, or immunoglobulins; hydrophilic polymers such as polyvinylpyrrolidone; amino acids, such as glycine, glutamic acid, aspartic acid, or arginine; monosaccharides, disaccharides, and other carbohydrates including cellulose or its derivatives, glucose, mannose, or dextrins; chelating agents such as EDTA; sugar alcohols such as mannitol or sorbitol; counterions such as sodium; and/or non-ionic surfactants such as polysorbates, poloxamers, or PEG.

GH is typically formulated individually in such vehicles at a concentration of about 0.1 mg/mL to 100 mg/mL, preferably 1-10 mg/mL, at a pH of about 4.5 to 8. GH is preferably at a pH of 7.4-7.8. It will be understood that use of certain of the foregoing excipients, carriers, or stabilizers will result in the formation of GH salts.

While GH can be formulated by any suitable method, the preferred formulations for GH are as follows: for a preferred hGH (GENOTROPIN™), a single-dose syringe contains 0.2 mg, 0.4 mg, 0.6 mg, 0.8 mg, 1.0 mg, 1.2 mg, 1.4 mg, 1.6 mg, 1.8 mg or 2.0 mg recombinant somatropin. Said GENOTROPIN™ syringe also contains 0.21 mg glycine, 12.5 mg mannitol, 0.045 mg monoatriumphosphate, 0.025 mg disodium phosphate and water to 0.25 ml.

For met-GH (PROTROPIN™), the pre-lyophilized bulk solution contains 2.0 mg/mL met-GH, 16.0 mg/mL mannitol, 0.14 mg/mL sodium phosphate, and 1.6 mg/mL sodium phosphate (monobasic monohydrate), pH 7.8. The 5-mg vial of met-GH contains 5 mg met-GH, 40 mg mannitol, and 1.7 mg total sodium phosphate (dry weight) (dibasic anhydrous), pH 7.8. The 10-mg vial contains 10 mg met-GH, 80 mg mannitol, and 3.4 mg total sodium phosphate (dry weight) (dibasic anhydrous), pH.7.8.

For metless-GH (NUTROPIN™), the pre-lyophilized bulk solution contains 2.0 mg/mL GH, 18.0 mg/mL mannitol, 0.68 mg/mL glycine, 0.45 mg/mL sodium phosphate, and 1.3 mg/mL sodium phosphate (monobasic monohydrate), pH 7.4. The 5-mg vial contains 5 mg GH, 45 mg mannitol, 1.7 mg glycine, and 1.7 mg total sodium phosphates (dry weight) (dibasic anhydrous), pH 7.4. The 10-mg vial contains 10 mg GH, 90 mg mannitol, 3.4 mg glycine, and 3.4 mg total sodium phosphates (dry weight) (dibasic anhydrous).

Alternatively, a liquid formulation for NUTROPIN™ hGH can be used, for example: 5.0.+−.0.5 mg/mL rhGH; 8.8.+−.0.9 mg/mL sodium chloride; 2.0.+−.0.2 mg/nL Polysorbate 20; 2.5.+−.0.3 mg/mL phenol; 2.68.+−.0.3 mg/mL sodium citrate dihydrate; and 0.17.+−.0.02 mg/mL citric acid anhydrous (total anhydrous sodium citrate/citric acid is 2.5 mg/mL, or 10 mM); pH 6.0.+−.0.3. This formulation is suitably put in a 10-mg vial, which is a 2.0-mL fill of the above formulation in a 3-cc glass vial. Alternatively, a 10-mg (2.0 mL) cartridge containing the above formulation can be placed in an injection pen for injection of liquid GH to the subject.

GH compositions to be used for therapeutic administration are preferably sterile. Sterility is readily accomplished by filtration through sterile filtration membranes (e.g., 0.2 micron membranes). Therapeutic GH compositions generally are placed into a container having a sterile access port, for example, an intravenous solution bag or vial having a stopper pierceable by a hypodermic injection needle.

The GH ordinarily will be stored in unit or multi-dose containers, for example, sealed ampoules or vials, as an aqueous solution, or as a lyophilized formulation for reconstitution. As an example of a lyophilized formulation, vials are filled with sterile-filtered it (w/v) aqueous GH solutions, and the resulting mixture is lyophilized. The infusion solution is prepared by reconstituting the lyophilized GH using bacteriostatic Water-for-Injection.

Drug Screening Assays

The discovery that individuals carrying a GHRd3 allele have increased positive response to treatment with an agent acting via the GHR pathway compared to individuals homozygous for the GHRfl allele has provided assays that can be used to evaluate therapeutic agents acting via the GHR pathway. For example, screening assays based on GHRd3 in which GHR activity or binding to GHRd3 is assessed can be used to identify agents that will be most useful for treating individuals expressing a GHRd3 allele. As further described below, agent that can be tested include agents known to be useful for the treatment of disease such as GH compositions including somatotropin or somatropin, preferably GENOTROPIN™, or PROTROPIN™, NUTROPIN™, or pegvisomant, preferably SOMAVERT™, or agents not yet known to be useful for the treatment of disease.

In one aspect, the invention provides a cell-based assay in which a cell which expresses a GHRd3 protein, or biologically active portion thereof, is contacted with a test compound and the ability of the test compound to modulate GHR activity is determined. Determining the ability of the test compound to modulate (e.g. stimulate or inhibit) GHR activity can be accomplished by monitoring the activity of the GHR polypeptide. Detecting GHR activity may comprise assessing any suitable detectable activity, including for example test compound-induced cell proliferation, GHR internalization and/or signal transduction, as further discussed below.

In preferred embodiments, the invention provides a method of identifying a candidate GHR modulator (e.g. agonist or antagonist), said method comprising a) providing a cell comprising a GHRd3 polypeptide; b) contacting said cell with a test compound; and c) determining whether said compound selectively stimulates or inhibits GHR activity. In one embodiment, the method comprises a) providing a human cell (preferably a 293 cell); b) introducing a vector comprising a nucleic acid sequence encoding GHRd3 polypeptide into said cell, and c) contacting said cell with a test compound; and d) detecting GHR activity. A detection that said compound inhibits GHR activity indicates that said compound is a candidate GHRd3 inhibitor. A detection that said compound stimulates GHR activity indicates that said compound is a candidate GHRd3 agonist. In another example the method comprises a) providing a Xenopus laevis oocyte; b) introducing GHRd3 cRNA into said Xenopus oocyte; c) contacting said Xenopus oocyte with a test compound; and d) detecting GHR activity in said Xenopus oocyte. Again, detection that said compound stimulates GHR activity indicates that said compound is a candidate GHRd3 agonist. Detection that said compound inhibits GHR activity indicates that said compound is a candidate GHRd3 antagonist. Further details of screening assays are described below in the context of GHRd3/fl heterodimers.

### GHRd3/GHRfl Heterodimers

Methods of Assessing GHRd3/fl Heterodimer Activity

As discussed, the invention provides that a GHRd3 polypeptide may exist naturally as a heterodimer with a GHRfl polypeptide. Thus, the invention provides methods for assessing the activity of GHRd3 polypeptides. In preferred aspects, the invention comprises detecting activity of a polypeptide complex comprising a GHRd3 polypeptide and a GHRfl polypeptide. The invention thus provides method of assessing the activity of a GHR polypeptide complex comprising a GHRd3 polypeptide. Preferably, the complex is a complex comprising a GHRd3 polypeptide, a GHRfl polypeptide, and a GH polypeptide.

The invention further provides methods of testing the activity of, or obtaining, functional variant GHRd3 nucleotide sequences involving providing a variant or modified GHRd3 nucleic acid and assessing whether a polypeptide encoded thereby displays GHR activity. Encompassed is thus a method of assessing the function of a GHRd3 polypeptide comprising: (a) providing a GHRd3 polypeptide and a GHRfl polypeptide; and (b) assessing GHR activity. Any suitable format may be used, including a cell free (e.g. membrane-based), cell-based and in vivo formats. For example, said assay may comprise expressing a GHRd3 and a GHRfl nucleic acid in a host cell, and observing GHR activity in said cell. In another example, a GHRd3 and a GHRfl polypeptide are introduced to a cell, and GHR activity is observed. In another example, a GHRd3 polypeptide is introduced to a cell which expresses a GHRfl polypeptide, and GHR activity is observed.

In preferred embodiments, detecting GHR activity may comprise determining the ability of the GHR protein to further modulate the activity of a downstream effector (e.g., a GHR-mediated signal transduction pathway component). For example, the activity of the effector molecule on an appropriate target can be determined or the binding of the effector to an appropriate target can be determined as previously described. Preferably Jak-2/Stat-5 signaling is assessed. In other preferred embodiments, detecting GHR activity may also comprise assessing any suitable detectable activity, including GHR ligand-induced cell proliferation, binding of GHR to a GHR ligand, GHR and/or ligand internalization. Most preferably, said GHR ligand is a GH polypeptide. These methods allow testing of activity of a GHR heterodimer comprised of a GHRd3 and a GHRfl polypeptide.

The methods of assessing GHRd3 activity may be useful for characterizing modified GHRd3 polypeptides. For example, GHRd3 polypeptides having a mutation at an essential or non-essential amino acid residue can be characterized. Nucleotide substitutions leading to amino acid substitutions at “non-essential” amino acid residues can be made in the sequences of GHRd3. A “non-essential” amino acid residue is a residue that can be altered from the wild-type sequence of a GHRd3 polypeptide without altering the biological activity, whereas an “essential” amino acid residue is required for biological activity. For example, amino acid residues that are conserved among the GHRd3 proteins of the present invention are predicted to be less amenable to alteration. Furthermore, additional conserved amino acid residues may be amino acids that are conserved between the GHRd3 proteins of the present invention. In other examples, for naturally-occurring allelic variants of the GHRd3 sequences that may exist in the population, changes can be introduced by mutation into the nucleotide sequences of a GHRd3 nucleic acid, thereby leading to changes in the amino acid sequence of the encoded GHRd3 proteins, with or without altering the functional ability of the GHRd3 proteins.

Drug Screening Assays

The invention provides methods for identifying and/or assessing GHR agonists and antagonists, i.e., candidate or test compounds or agents (e.g., preferably polypeptides, but also peptides, peptidomimetics, small molecules or other drugs) which act via the GHR pathway. Preferably the GHR agonists and antagonists are compounds that bind to GHRd3 and GHRfl proteins thereby preferably forming a complex comprising a GHRd3 polypeptide, a GHRfl polypeptide and said compound. Assays may be cell based or non-cell based assays. Preferred non-cell based assays are membrane based assays. The assays may also be referred to herein as “screening assays”. Screening assays may be binding assays or other functional assays, as based on any suitable known GHR activity assays.

In one aspect, an assay is a cell-based assay in which a cell which expresses a GHRd3 protein and a GHRfl protein, or biologically active portions thereof, is contacted with a test compound and the ability of the test compound to modulate GHR activity is determined. Determining the ability of the test compound to modulate (e.g. stimulate or inhibit) GHR activity can be accomplished by monitoring the activity of the GHR polypeptide (e.g. the GHR polypeptide complex comprising GHRd3 and GHRfl proteins). Detecting GHR activity may comprise assessing any suitable detectable activity, including for example test compound-induced cell proliferation, GHR internalization and/or signal transduction.

In preferred embodiments, the invention provides a method of identifying a candidate GHR modulator (e.g. agonist or antagonist), said method comprising a) providing a cell comprising a GHRd3 and a GHRfl polypeptide; b) contacting said cell with a test compound; and c) determining whether said compound selectively stimulates or inhibits GHR activity. In one embodiment, the method comprises a) providing a human cell (preferably a 293 cell); b) introducing a vector comprising a nucleic acid sequence encoding GHRd3 polypeptide into said cell, and optionally introducing a vector comprising a nucleic acid sequence encoding GHRfl polypeptide into said cell; c) contacting said cell with a test compound; and d) detecting GHR activity. A detection that said compound inhibits GHR activity indicates that said compound is a candidate GHRd3/GHRfl heterodimer inhibitor. A detection that said compound stimulates GHR activity indicates that said compound is a candidate GHRd3/GHRfl heterodimer agonist. In another example the method comprises a) providing a Xenopus laevis oocyte; b) introducing GHRd3 and optionally a GHRfl cRNA into said Xenopus oocyte; c) contacting said Xenopus oocyte with a test compound; and d) detecting GHR activity in said Xenopus oocyte. Again, detection that said compound stimulates GHR activity indicates that said compound is a candidate GHRd3/GHRfl heterodimer agonist. Detection that said compound inhibits GHR activity indicates that said compound is a candidate GHRd3/GHRfl heterodimer antagonist.

Detecting GHR activity may involve assessing any suitable detectable activity, including cell proliferation, binding of GHR to a GHR ligand (e.g. a GH polypeptide), GHR and/or GHR ligand internalization and/or GHR-mediated signal transduction.

An example of a GHR functional assay in 293 cells for GHR-mediated Jak2-Stat5 signaling is described in Maamra et al, (1999) J. Biol. Chem. 274:14791-14798, the disclosure of which is incorporated herein by reference. An example of a GHR functional assay in Xenopus laevis oocytes is described in Urbanek et al. (1993) J. Biol. Chem. 268(25): 19025-19032, the disclosure of which is incorporated herein by reference. Methods for generation of cDNA templates, in vitro transcription and translation, oocyte injection, analysis of injected mRNA stability, determining GH binding to GHR and analysis of receptor internalization can be carried out essentially as described in Urbanek et al. (1993).

In another preferred aspect, an assay is a cell-based binding assay in which a cell which expresses a GHRd3 protein and a GHRfl protein, or biologically active portions thereof, is contacted with a test compound and the ability of the test compound to bind a GHR polypeptide is determined. In another aspect, an assay is a non-cell-based binding assay in which a membrane comprising a GHRd3 protein and a GHRfl protein, or biologically active portions thereof, is contacted with a test compound and the ability of the test compound to bind a GHR polypeptide is determined. Determining the ability of the test compound to bind to a GHR (e.g. GHRd3 or GHRfl) polypeptide can be accomplished using known methods.

Bnding assays may for example comprise: a) providing a cell comprising a GHRd3 and a GHRfl polypeptide; b) contacting said cell with a test compound; and c) determining whether said compound selectively binds to a GHR polypeptide. In one embodiment, the method comprises a) providing an human cell (preferably a 293 cell); b) introducing a vector comprising a nucleic acid sequence encoding GHRd3 polypeptide into said cell, and optionally introducing a vector comprising a nucleic acid sequence encoding GHRfl polypeptide into said cell; c) contacting said cell with a test compound; and d) detecting whether said compound selectively binds to a GHR polypeptide. Detection that said compound bind to a GHR polypeptide indicates that said compound is a candidate GHRd3/GHRfl heterodimer modulator. In another example the method comprises a) providing a Xenopus laevis oocyte; b) introducing GHRd3 and optionally a GHRfl cRNA into said Xenopus oocyte; c) contacting said Xenopus oocyte with a test compound; and d) detecting whether said compound selectively binds to a GHR polypeptide. Again, detection that said compound binds a GHR polypeptide indicates that said compound is a candidate GHRd3/GHRfl heterodimer modulator.

An example of a 293 cell-based GHR binding assay is described in Ross et al, (2001) J. Clin. Endocrinol. Metabol. 86(4) 1716-1723, the disclosure of which is incorporated herein by reference.

Preferably the cells used in the above assays are 293 cells expressing a GHRfl polypeptide. 293 cells expressing GHR have been described in Maamra et al, (1999) J. Biol. Chem. 274:14791-14798, the disclosure of which is incorporated herein by reference.

Such assays can be particularly useful for testing GH polypeptides or fragments or variants thereof. Particularly preferred are GH polypeptides that have been modified so as to have longer blood circulation times, for example by the linking of polyethylene glycol molecules. In other embodiments, GH polypeptides may be GHR antagonists such as GH polypeptides which bind a GHR protein (e.g. preferably forming a complex comprising a GHRd3 and a GHRfl protein), but which do not stimulate GHR activity.

In another embodiment, the assay comprises contacting a cell which expresses a GHRd3 protein and a GHRfl protein or biologically active portion thereof, with a GHR ligand to form an assay mixture, contacting the assay mixture with a test compound, detecting GHR activity. Preferably, the method comprises determining the ability of the test compound to stimulate or inhibit activity of the GHR protein (e.g. the GHR dimer comprising GHRd3 and GHRfl protein or biologically active portions thereof), wherein determining the ability of the test compound to inhibit the activity of the GHR protein comprises determining the ability of the test compound to inhibit a biological activity of the GHRd3- and GHRfl-expressing cell (e.g., determining the ability of the test compound to inhibit signal transduction or protein:protein interactions).

Determining the ability of the GHR protein to bind to or interact with a GHR ligand can be accomplished by one of the methods described above for determining direct binding. In other embodiments, determining the ability of the GHRd3 protein or complex comprising a GHRd3 protein to bind to or interact with a GHR ligand molecule can be accomplished by detecting induction of a cellular second messenger of the target (i.e. intracellular Ca2+, diacylglycerol, IP₃, etc.), detecting a catalytic/enzymatic activity on an appropriate substrate, detecting the induction of a reporter gene (comprising a responsive regulatory element operatively linked to a nucleic acid encoding a detectable marker, e.g., luciferase), or detecting a GHR-regulated cellular response, for example, signal transduction or protein:protein interaction.

In yet another embodiment, an assay of the present invention is a cell-free assay in which a GHRd3 protein and a GHRfl protein, or a biologically active portion thereof are provided in a membrane, and GHR proteins or the membrane are contacted with a test compound and the ability of the test compound to bind to the GHR protein (e.g. GHRd3 and/or GHRfl proteins) or biologically active portion thereof is determined. Binding of the test compound to the GHRd3 protein can be determined either directly or indirectly as described above. In a preferred embodiment, the assay includes contacting the GHR protein (e.g. the GHR heterodimer) or biologically active portion thereof with a known compound such as a GH polypeptide which binds GHR to form an assay mixture, contacting the assay mixture with a test compound, and determining the ability of the test compound to interact with a GHR protein, wherein determining the ability of the test compound to interact with a GHR heterodimer protein comprises determining the ability of the test compound to preferentially bind to GHR protein or biologically active portion thereof as compared to the known compound.

In another embodiment, the assay is a cell-free assay in which a GHRd3 protein and a GHRfl protein or biologically active portion thereof are provided in a membrane, and the polypeptides or the membrane are contacted with a test compound and the ability of the test compound to modulate (e.g., stimulate or inhibit) the activity of the GHR protein or biologically active portion thereof is determined. Determining the ability of the test compound to modulate GHR activity can be accomplished, for example, by assessing any suitable GHR detectable activity, including cell proliferation, binding of GHR to a GHR ligand (e.g. a GH polypeptide), GHR and/or GHR ligand internalization and/or GHR-mediated signal transduction.

Determining the ability of the test compound to inhibit GHR activity can also be accomplished, for example, by coupling a test compound such as a GH molecule protein or a portion or derivative thereof with a radioisotope or enzymatic label such that binding of the GH molecule to a GHR heterodimer can be determined by detecting the labeled GH protein or biologically active portion thereof in a complex. For example, compounds (e.g., GH protein or biologically active portion thereof) can be labeled with ¹²⁵I, ³⁵S, ¹⁴C, or ³H, either directly or indirectly, and the radioisotope detected by direct counting of radioemmission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

It is also within the scope of this invention to determine the ability of a compound (e.g., GH protein or portion or fragment thereof) to interact with a GHR polypeptide (e.g. GHRd3/GHRfl heterodimer) without the labeling of any of the interactants. For example, a microphysiometer can be used to detect the interaction of a compound with its cognate target molecule without the labeling of either the compound or the receptor. McConnell, H. M. et al. (1992) Science 257:1906-1912. A microphysiometer such as a cytosensor is an analytical instrument that measures the rate at which a cell acidifies its environment using a light-addressable potentiometric sensor (LAPS). Changes in this acidification rate can be used as an indicator of the interaction between compound and receptor.

Determining the ability of the GHR protein to bind to a GHR ligand or test compound can also be accomplished using a technology such as real-time Biomolecular Interaction Analysis (BIA). Sjolander, S. and Urbaniczky, C. (1991) Anal. Chem. 63:2338-2345 and Szabo et al. (1995) Curr. Opin. Struct. Biol. 5:699-705. As used herein, “BIA” is a technology for studying biospecific interactions in real time, without labeling any of the interactants (e.g., BIAcore). Changes in the optical phenomenon of surface plasmon resonance (SPR) can be used as an indication of real-time reactions between biological molecules.

Test Compounds

The “candidate” or “test” compounds or “agents” (e.g. test GHR agonists and antagonists) may be of any suitable form, including polypeptides, peptides, peptidomimetics, small molecules and other drugs. Said compounds or agents include those known to be useful for the treatment of disease, or agents not yet known to be useful for the treatment of disease.

In a preferred embodiment, a test compound is a GH polypeptide. A preferred GH may be in native-sequence or in variant form, and from any source, whether natural, synthetic, or recombinant. Examples include human growth hormone (hGH), which is natural or recombinant GH with the human native sequence, and recombinant growth hormone (rGH), which refers to any GH or GH variant produced by means of recombinant DNA technology. In one aspect the GH is capable of stimulating the GHR receptor; examples include somatotropin or somatropin, preferably GENOTROPIN™, or PROTROPIN™, NUTROPIN™.

In another aspect, the GH polypeptide is a GH variant capable of acting as a GHR antagonist. GHR antagonists are a class of drugs intended to bind GHR polypeptides but to block GHR function. One example of a GHR antagonist is a described in Ross et al, (2001) J. Clin. Endocrinol. Metabol. 86(4) 1716-1723. This GHR antagonist disclosed in Ross et al., referred to as B2036-PEG, is a pegylated GH variant polypeptide having mutations in site 1 to enhance GHR binding and in site 2 to block receptor dimerization. A preferred GHR antagonist or inhibitor is pegvisomant, preferably SOMAVER™. GHR antagonists are useful for the treatment of acromegaly, a condition usually caused by excessive GH secretion from a pituitary adenoma.

The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is used with peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc Natl Acad Sci USA 89:1865-1869) or on phage (Scott and Smith (1990) Science 249:386-390); (Devin (1990) Science 249:404406); (Cwirla et al. (1990) Proc. Natl. Acad. Sci. 87:6378-6382); (Felici (1991) J. Mol. Biol. 222:301-310); (Ladner supra.).

This invention further pertains to novel agents identified by the above-described screening assays and to processes for producing such agents by use of these assays. Accordingly, in one embodiment, the present invention includes a compound or agent obtainable by a method comprising the steps of any one of the aformentioned screening assays (e.g., cell-based assays or cell-free assays). Preferably, said compound or agent comprises a GH polypeptide, or a portion or variant thereof.

Accordingly, it is within the scope of this invention to further use an agent identified as described herein in an appropriate animal model. For example, an agent identified as described herein (e.g., a GHR modulating agent such as a GH polypeptide or portion or variant thereof, an antisense GHRd3 nucleic acid molecule, a GHRd3-specific antibody, or a GHRd3-binding partner) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein.

The present invention also pertains to uses of novel agents identified by the above-described screening assays for diagnoses, prognoses, and treatments as described herein. Accordingly, it is within the scope of the present invention to use such agents in the design, formulation, synthesis, manufacture, and/or production of a drug or pharmaceutical composition for use in diagnosis, prognosis, or treatment, as described herein. For example, in one embodiment, the present invention includes a method of synthesizing or producing a drug or pharmaceutical composition by reference to the structure and/or properties of a compound obtainable by one of the above-described screening assays. For example, a drug or pharmaceutical composition can be synthesized based on the structure and/or properties of a compound obtained by a method in which a cell which expresses GHRd3 and GHRfl polypeptides is contacted with a test compound and the ability of the test compound to bind to, or modulate the activity of, the GHR polypeptide (preferably a complex comprising a GHRd3 and a GHRfl polypeptide) is determined. In another exemplary embodiment, the present invention includes a method of synthesizing or producing a drug or pharmaceutical composition based on the structure and/or properties of a compound obtainable by a method in which a GHRd3 protein or biologically active portion thereof is contacted with a test compound and the ability of the test compound to bind to, or modulate (e.g., stimulate or inhibit) the activity of, a GHR protein, preferably a GHR dimer comprising a GHRd3 and GHRfl protein, or biologically active portions thereof is determined.

GHRd3 Nucleic Acids and Proteins

As discussed herein, the invention relates to the use of GHRd3 nucleic acids and polypeptides.

In a preferred embodiment, the GHRd3 protein comprises a contiguous span of at least 6 amino acids, preferably at least 8 to 10 amino acids, more preferably at least 12, 15, 20, 25, 30, 40, 50, 100, 200, 300, 400, 500 or 600 amino acids. In other preferred embodiments the contiguous stretch of amino acids comprises the site of a mutation or functional mutation, including a deletion, addition, swap or truncation of the amino acids in the GHRd3 protein sequence. Thus, also useful in the context of the present invention are biologically active portions of a GHRd3 protein include peptides comprising amino acid sequences sufficiently homologous to or derived from the amino acid sequence of the GHRd3 protein, which include less amino acids than the full length GHRd3 proteins, and exhibit at least one activity of a GHR protein. In other embodiments, a GHRd3 protein is substantially homologous to the native GHRd3 sequence and retains the functional activity of the native GHRd3 protein, yet differs in amino acid sequence due to natural allelic variation or mutagenesis. Accordingly, in another embodiment, the GHRd3 protein is a protein which comprises an amino acid sequence at least about 60% homologous to the amino acid sequence described in (Urbanek et al. (1992) and retains the functional activity of the GHRd3 protein, respectively. Preferably, the protein is at least about 30%, 40%, 50%, 60%, 70%, 80%, 85%, 90%, 92%, 95%, 97%, 98%, 99% or 99.8% homologous to Urbanek et al. (1992).

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, 90% or 95% of the length of the reference sequence (e.g., when aligning a second sequence to the GHRd3 amino acid sequence, at least 100, preferably at least 200 amino acid residues are aligned). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “identity” is equivalent to amino acid or nucleic acid “homology”). The percent homology between the two sequences is a function of the number of identical positions shared by the sequences (i.e., % homology=# of identical positions/total # of positions*100).

The comparison of sequences and determination of percent homology between two sequences can be accomplished using a mathematical algorithim. A preferred, non-limiting example of a mathematical algorithim utilized for the comparison of sequences is the algorithm of Karlin and Altschul (1990) Proc. Natl. Acad. Sci. USA 87:2264-68, modified as in Karlin and Altschul (1993) Proc. Natl. Acad. Sci. USA 90:5873-77. Such an algorithm is incorporated into the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to GHRd3 nucleic acid molecules of the invention. BLAST protein searches can be performed with the XBLAST program, score=50, wordlength=3 to obtain amino acid sequences homologous to GHRd3 protein molecules of the invention. To obtain gapped alignments for comparison purposes, Gapped BLAST can be utilized as described in Altschul et al., (1997) Nucleic Acids Research 25(17):3389-3402. When utilizing BLAST and Gapped BLAST programs, the default parameters of the respective programs (e.g., XBLAST and NBLAST) can be used. See http://www.ncbi.nlm.nih.gov. Another preferred, non-limiting example of a mathematical algorithim utilized for the comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

Recombinant Expression Vectors and Host Cells

Vectors, preferably expression vectors, containing a nucleic acid encoding a GHRd3 protein (or a portion thereof) can be prepared according to any suitable method. Likewise, in preferred aspects, expression vectors comprising a nucleic acid encoding a GHRfl protein (or a portion thereof) can also be prepared. Optionally, an expression vector will comprise a nucleic acid that encodes a GHRd3 protein as well as a nucleic acid that encodes a GHRfl protein. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a GHRd3 and/or GHRfl nucleic acid in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to includes promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors can be introduced into host cells to thereby produce proteins or peptides.

The recombinant expression vectors of the invention can be designed for expression of GHRd3 proteins in prokaryotic or eukaryotic cells. For example, GHRd3 proteins can be expressed in bacterial cells such as E. coli, insect cells (using baculovirus expression vectors) yeast cells, or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:3140), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Examples of suitable inducible non-fusion E. coli expression vectors include pTrc (Amann et al., (1988) Gene 69:301-315) and pET 11d (Studier et al., Gene Expression Technology: Methods in Enzymology 185, Academic Press, San Diego, Calif. (1990) 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn 1). This viral polymerase is supplied by host strains BL21 (DE3) or HMS174(DE3) from a resident prophage harboring a T7 gn1 gene under the transcriptional control of the lacUV 5 promoter.

One strategy to maximize recombinant protein expression in E. coli is to express the protein in a host bacteria with an diminished capacity to proteolytically cleave the recombinant protein (Gottesman, S., Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in E. coli (Wada et al., (1992) Nucleic Acids Res. 20:2111-2118). Such alteration of nucleic acid sequences of the invention can be carried out by standard DNA synthesis techniques.

In another embodiment, the GHRd3 expression vector is a yeast expression vector. Examples of vectors for expression in yeast S. cerivisae include pYepSec 1 (Baldari, et al., (1987) Embo J. 6:229-234), pMFa (Kurjan and Herskowitz, (1982) Cell 30:933-943), pJRY88 (Schultz et al., (1987) Gene 54:113-123), pYES2 (Invitrogen Corporation, San Diego, Calif.), and picZ (InVitrogen Corp, San Diego, Calif.).

Alternatively, GHRd3 proteins can be expressed in insect cells using baculovirus expression vectors. Baculovirus vectors available for expression of proteins in cultured insect cells (e.g., Sf 9 cells) include the pAc series (Smith et al. (1983) Mol. Cell Biol. 3:2156-21.65) and the pVL series (Lucklow and Summers (1989) Virology 170:31-39). In particularly preferred embodiments, GHRd3 proteins are expressed according to Karniski et al, Am. J. Physiol. (1998) 275: F79-87.

In yet another embodiment, a nucleic acid of the invention is expressed in mammalian cells using a mammalian expression vector. Examples of mammalian expression vectors include pCDM8 (Seed, B. (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989.

Another aspect of the invention pertains to host cells into which a recombinant expression vector of the invention has been introduced. The terms “host cell” and “recombinant host cell” are used interchangeably herein. It is understood that such term refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a GHRd3 protein can be expressed in bacterial cells such as E. coli, insect cells, yeast or mammalian cells (preferably human 293 cells). Other suitable host cells are known to those skilled in the art, including Xenopus laevis oocytes.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (Molecular Cloning: A Laboratory Manual. 2nd, ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin and methotrexate. Nucleic acid encoding a selectable marker can be introduced into a host cell on the same vector as that encoding a GHRd3 protein or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) a GHRd3 protein. Accordingly, the invention further provides methods for producing a GHRd3 protein using the host cells of the invention. In one embodiment, the method comprises culturing the host cell of invention (into which a recombinant expression vector encoding a GHRd3 protein has been introduced) in a suitable medium such that a GHRd3 protein is produced.

The host cells of the invention can also be used to produce nonhuman transgenic animals homozygous or heterozygous for GHRd3 allele. For example, in one embodiment, a host cell of the invention is a fertilized oocyte or an embryonic stem cell into which GHRd3-coding sequences have been introduced. Such host cells can then be used to create non-human transgenic animals in which exogenous GHRd3 sequences have been introduced into their genome or homologous recombinant animals in which endogenous GHRfl sequences have been altered. Such animals are useful for studying the function and/or activity of a GHRd3 and for identifying and/or evaluating modulators of GHRd3 activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous GHR gene (e.g. GHRfl allele) has been altered by homologous recombination between the endogenous gene and an exogenous DNA molecule introduced into a cell of the animal, e.g., an embryonic cell of the animal, prior to development of the animal.

A transgenic animal of the invention can be created by introducing a GHRd3-encoding nucleic acid into the male pronuclei of a fertilized oocyte, e.g., by microinjection, retroviral infection, and allowing the oocyte to develop in a pseudopregnant female foster animal. A GHRd3 cDNA sequence can be introduced as a transgene into the genome of a non-human animal. Intronic sequences and polyadenylation signals can also be included in the transgene to increase the efficiency of expression of the transgene. A tissue-specific regulatory sequence(s) can be operably linked to a GHRd3 transgene to direct expression of a GHRd3 protein to particular cells. Methods for generating transgenic animals via embryo manipulation and microinjection, particularly animals such as mice, have become conventional in the art and are described, for example, in U.S. Pat. Nos. 4,736,866 and 4,870,009, both by Leder et al., U.S. Pat. No. 4,873,191 by Wagner et al. and in Hogan, B., Manipulating the Mouse Embryo, (Cold Spring Harbor Laboratory Press, Cold Spring Harbor, N.Y., 1986). Similar methods are used for production of other transgenic animals. A transgenic founder animal can be identified based upon the presence of a GHRd3 transgene in its genome and/or expression of GHRd3 mRNA in tissues or cells of the animals. A transgenic founder animal can then be used to breed additional animals carrying the transgene. Moreover, transgenic animals carrying a transgene encoding a GHRd3 protein can further be bred to other transgenic animals carrying other transgenes.

To create a homologous recombinant animal, a vector is prepared which contains at least a portion of a GHRd3 nucleic acid to thereby alter the endogenous GHR (GHRfl) gene. The GHRd3 gene can be a human gene or a non-human homologue of a human GHR gene (e.g., a cDNA isolated by stringent hybridization with a nucleotide sequence derived from SEQ ID NO:1, 4 or 6). Preferably the non-human homolog is generated by a modification of the non-human GHR sequence to delete exon 3 nucleic acids. Since the GHRd3 allele is not observed in mice for example, a mouse GHRd3 nucleic acid may be prepared using known methods. Thus, in certain aspects, a synthetic mouse GHRd3 gene can be used in a homologous recombination vector suitable for altering an endogenous GHR gene in the mouse genome. In a preferred embodiment, the vector is designed such that, upon homologous recombination, the endogenous GHR gene is replaced by a GHRd3 gene, said GHRd3 gene encoding a functional GHRd3 protein (e.g., the upstream regulatory region can be altered to thereby alter the expression of the endogenous GHRd3 protein). In the homologous recombination vector, the GHRd3 gene is flanked at its 5′ and 3′ ends by additional nucleic acid sequence of the GHR gene to allow for homologous recombination to occur between the exogenous GHRd3 gene carried by the vector and an endogenous GHR gene in an embryonic stem cell. The additional flanking GHR nucleic acid sequence is of sufficient length for successful homologous recombination with the endogenous gene. Typically, several kilobases of flanking DNA (both at the 5′ and 3′ ends) are included in the vector (see e.g., Thomas, K. R. and Capecchi, M. R. (1987) Cell 51:503 for a description of homologous recombination vectors). The vector is introduced into an embryonic stem cell line (e.g., by electroporation) and cells in which the introduced GHRd3 gene has homologously recombined with the endogenous GHR gene are selected (see e.g., Li, E. et al. (1992) Cell 69:915). The selected cells are then injected into a blastocyst of an animal (e.g., a mouse) to form aggregation chimeras (see e.g., Bradley, A. in Teratocarcinomas and Embryonic Stem Cells. A Practical Approach, E. J. Robertson, ed. (IRL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley, A. (1991) Current Opinion in Biotechnology 2:823-829 and in PCT International Publication Nos.: WO 90/11354 by Le Mouellec et al.; WO 91/01140 by Smithies et al.; WO 92/0968 by Zijlstra et al.; and WO 93/04169 by Berns et al.

In another embodiment, transgenic non-humans animals can be produced which contain selected systems which allow for regulated expression of the transgene. One example of such a system is the cre/loxP recombinase system of bacteriophage P1. For a description of the cre/loxP recombinase system, see, e.g., Lakso et al. (1992) PNAS 89:6232-6236. Another example of a recombinase system is the FLP recombinase system of Saccharomyces cerevisiae (O'Gorman et al. (1991) Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

The invention will be more fully understood by reference to the following examples. They should not, however, be construed as limiting the scope of the invention. All literature and patent citations are expressly incorporated herein by reference.

### EXAMPLES

### Example 1

### PCR RFLP Genotyping for GHRd3 and GHRfl

PCR RFLP

PCR amplification was performed in 96-well microtiter plates (Perkin Elmer), each well containing 50 μl of reaction mixture containing 200 ng DNA, 1.5 mM MgCl₂, 5 μl 10× reaction Buffer (Perkin Elmer), 0.2 mM each dNTP, 0.2 μM of each primer and 1.25 U of Taq Polymerase (Perkin Elmer).

35 PCR cycles were performed using a 9700 Perkin Elmer thermocycler. PCR products were detected on agarose gel.

| | Annealing | | Method of |
|---|---|---|---|
| Primers (5′-3′) | Temp | PCR product | detection |
| G1: TGTGCTGGTCTGTTGGTCTG | 60° C. | fl/fl: 935 bp | 1% agarose gel |
| G2: AGTCGTTCCTGGGACAGAGA | | fl/d3: 935, and 532 bp | in 0.5X TBE |
| G3: CCTGGATTAACACTTTGCAGACTC | | d3/d3: 532 bp | |

### Example 2

### Detection of GHRd3 Allele Associated with GH Response

97 Children with idiopathic short stature (ISS) who had been enrolled in trials for treatment with recombinant GH were examined for association of the common GHR exon 3 variant and the response of growth velocity to treatment with GH. The GHRd3 allele was present in 47 patients, of which 3 were GHRd3/d3 homozygotes and 44 were GHRd3/fl heterozyotes, as shown in Table 1.

TABLE 1
GHR genotype distribution in Caucasian individuals

| | Short children | Normal controls |
|---|---|---|
| f1/f1 | 50 | 210 |
| d3/f1 | 44 | 181 |
| d3/d3 | 3 | 12 |

Included were patients having normal short stature. Excluded were: patients who were ‘truly’ deficient in growth hormone (GHD), patients having CNS tumors, patients having mutations in the GHR gene, patients having other hormone deficits, patients having Turner syndrome, PHP, hypochondroplasia or other bone dysplasia, Laron's disease or other disease. Finally, the patients included did not show pubertal signs (breast, testes) at the termination of GH treatment after 2 years.

The genotypic groups were comparable with respect to other medical and therapeutic characteristics. Patients characteristics including age, sex, dose of rGH and size at birth and parental heights were taken into account (Tables 2, 3 and 4).

TABLE 2
Clinical and Biological Phenotypes at entry

GHR genotypes

| | f1/f1 | f1/d3 or d3/d3 |
|---|---|---|
| N | 50 | 47 |
| Age (yrs) | 7.63 ± 0.32 | 7.80 ± 0.30 |

Sex

| | 29M/21F | 32M/15F |
|---|---|---|
| GH peak (ng/ml) | | |
| O | 8.8 ± 0.9 (31) | 9.2 ± 1.5 (28) |
| AI | 8.2 ± 1.3 (23) | 8.1 ± 1.2 (17) |
| GBX | 10.8 ± 2.0 (28) | 10.0 ± 1.5 (30) |
| Others | 8.7 ± 1.9 (18) | 6.9 ± 1.1 (17) |

TABLE 3
GH posology in the two genotypic groups

| | f1/f1 | f1/d3, d3/d3 |
|---|---|---|
| rGH dose Year 1 (U.kg.w) | 0.714 ± 0.055 | 0.727 ± 0.066 |
| rGH dose Year 2 (U.kg.w) | 0.712 ± 0.056 | 0.727 ± 0.600 |

TABLE 4
Size at birth and Parental heights

GHR genotypes

| | f1/f1 | d1/f1 or d3/d3 |
|---|---|---|
| n: | 50 | 47 |
| Birth Ht (cm): | 47.2 ± 0.4 | 47.6 ± 0.4 |
| Birth Wt (g): | 2885 ± 90 | 2929 ± 85 |
| Father Ht (cm): | 170 ± 1.2 | 169.2 ± 0.8 |
| Mother Ht (cm): | 157.2 ± 0.9 | 157.3 ± 0.9 |

Growth rates were followed for a 2-year period during treatment with rhGH (Table 5). After adjustment for age, sex, dose of rGH, children who carried the GHRd3 variant grew at a superior rate when treated with rGH. Growth velocity was 9.0+/−0.3 cm/yr the first year of therapy and 7.8+/−0.2 cm/yr the second year in children with GHRd3/fl or GHRd3/d3 genotypes, compared with 7.4+/−0.2 and 6.5+/−0.2 cm/yr, respectively, in children with GHRfl/fl genotypes (P<0.0001). The genomic variation of the GHR sequence is therefore associated with a marked difference in rGH efficiency.

TABLE 5
Growth rates in the two genotypic groups

| | | f1/d3 or | |
|---|---|---|---|
| | f1/f1 | d3/d3 | P |
| | 50 | 47 | |
| Growth velocity at onset (cm/yr): | 4.83 ± 0.11 | 4.30 ± 0.15 | |
| Year 1 | 7.39 ± 0.20 | 9.01 ± 0.30 | <0.0001 |
| Year 2 | 6.48 ± 0.19 | 7.77 ± 0.24 | <0.0001 |
| Corrected Y1 - 0 (cm/yr): | 2.55 ± 0.24 | 4.72 ± 0.38 | <0.0001 |
| Corrected Y2 - 0 (cm/yr): | 1.65 ± 0.21 | 3.47 ± 0.32 | <0.0001 |

For multivariate analysis of growth rates, a mixed linear model was used to model the correlation between intra-individual measures (Table 6). The covariates considered included age, sex, growth velocity (VCO) before treatment with recombinant GH (horse riding effect), height at onset, GH dose GHR genotype, and subject (intercept).

The results showed that the rate of growth of patients with the exon 3 deleted GHR (GHRd3) is superior to patients homozygous for the full-length GHR isoform (GHRfl) after adjustment on the covariates. The correlation matrix shows that this effect is independent from the other covariates.

TABLE 6
Linear Regression Model for various parameters

Multiple regression

| | t-value | p-value |
|---|---|---|
| Age | −5.308 | <0.0001 |
| GV0 | −3.017 | 0.0033 |
| Sex | 3.495 | 0.0007 |
| rGH dose | 3.389 | 0.001 |
| GHR genotype | 4.520 | <0.0001 |

1. A method of predicting a subject's response to an agent capable of binding to a GHR protein, comprising determining in the subject the presence or absence of an allele of the GHR gene, wherein the allele is correlated with a likelihood of having an increased or decreased positive response to said agent, thereby identifying the subject as having an increased or decreased likelihood of responding to treatment with said agent.

2. A method of predicting a subject's response to an agent for increasing the height or growth rate of a subject, comprising determining in the subject the presence or absence of an allele of the GHR gene, wherein the allele is correlated with a likelihood of having an increased or decreased positive response to said agent, thereby identifying the subject as having an increased or decreased likelihood of responding to treatment with said agent.

3. A method of predicting a subject's response to an agent for treating obesity, comprising determining in the subject the presence or absence of an allele of the GHR gene, wherein the allele is correlated with a likelihood of having an increased or decreased positive response to said agent, thereby identifying the subject as having an increased or decreased likelihood of responding to treatment with said agent.

4. A method of predicting a subject's response to an agent for treating infection, comprising determining in the subject the presence or absence of an allele of the GHR gene, wherein the allele is correlated with a likelihood of having an increased or decreased positive response to said agent, thereby identifying the subject as having an increased or decreased likelihood of responding to treatment with said agent.

5. A method of predicting a subject's response to an agent for treating diabetes, comprising determining in the subject the presence or absence of an allele of the GHR gene, wherein the allele is correlated with a likelihood of having an increased or decreased positive response to said agent, thereby identifying the subject as having an increased or decreased likelihood of responding to treatment with said agent.

6. A method of predicting a subject's response to an agent for treating an acromegaly or gigantism condition, comprising determining in the subject the presence or absence of an allele of the GHR gene, wherein the allele is correlated with a likelihood of having an increased or decreased positive response to said agent, thereby identifying the subject as having an increased or decreased likelihood of responding to treatment with said agent.

7. A method of predicting a subject's response to an agent for treating a conditions associated with sodium or water retention, comprising determining in the subject the presence or absence of an allele of the GHR gene, wherein the allele is correlated with a likelihood of having an increased or decreased positive response to said agent, thereby identifying the subject as having an increased or decreased likelihood of responding to treatment with said agent.

8. A method of predicting a subject's response to an agent for treating a metabolic syndrome, comprising determining in the subject the presence or absence of an allele of the GHR gene, wherein the allele is correlated with a likelihood of having an increased or decreased positive response to said agent, thereby identifying the subject as having an increased or decreased likelihood of responding to treatment with said agent.

9. A method of predicting a subject's response to an agent for treating a mood or sleep disorders, comprising determining in the subject the presence or absence of an allele of the GHR gene, wherein the allele is correlated with a likelihood of having an increased or decreased positive response to said agent, thereby identifying the subject as having an increased or decreased likelihood of responding to treatment with said agent.

10. A method of predicting a subject's response to an agent for treating cancer, comprising determining in the subject the presence or absence of an allele of the GHR gene, wherein the allele is correlated with a likelihood of having an increased or decreased positive response to said agent, thereby identifying the subject as having an increased or decreased likelihood of responding to treatment with said agent.

11. A method of predicting a subject's response to an agent for treating cardiac disease, comprising determining in the subject the presence or absence of an allele of the GHR gene, wherein the allele is correlated with a likelihood of having an increased or decreased positive response to said agent, thereby identifying the subject as having an increased or decreased likelihood of responding to treatment with said agent.

12. A method of predicting a subject's response to an agent for treating hypertension, comprising determining in the subject the presence or absence of an allele of the GHR gene, wherein the allele is correlated with a likelihood of having an increased or decreased positive response to said agent, thereby identifying the subject as having an increased or decreased likelihood of responding to treatment with said agent.

13. A method of identifying a subject having an increased or decreased likelihood of treating a disorder or condition with an agent capable of binding to a GHR protein, comprising:
a) correlating the presence of an allele of the GHR gene with a subject's response to an agent capable of binding to a GHR protein; and
b) detecting the allele of step a) in the subject, thereby identifying a subject an increased or decreased likelihood of responding to treatment with said agent.

14. A method of identifying an allele in the GHR gene correlated with an increased or decreased likelihood of treating a disorder or condition with an agent capable of binding to a GHR protein, comprising:
a) determining in a subject the presence of an allele of the GHR gene; and
b) correlating the presence of the allele of step (a) with an increased or decreased likelihood of treating a disorder or condition with an agent capable of binding to a GHR protein, thereby identifying an allele correlated with an increased or decreased likelihood of responding to treatment with said agent.

15. The method of claims 1 or 13 to 14, wherein said agent capable of binding to a GHR protein is an agent capable of treating a disorder selected from the group consiting of: short stature, obesity, infection, or diabetes; acromegaly or gigantism conditions, or a lactogenic, diabetogenic, lipolytic and protein anabolic effect associated therewith; condition associated with sodium or water retention; metabolic syndromes; mood and sleep disorders, cancer, cardiac disease and hypertension.

16. A method for increasing the growth of a subject, the method comprising:
(a) determining in the subject the presence or absence of an allele of the GHR gene, wherein the allele is correlated with a likelihood of having an increased or decreased positive response to an agent capable of increasing the growth of a subject; and
(b) selecting or determining an effective amount of said agent to administer to said subject.

17. A method for treating a subject suffering from obesity, the method comprising:
(a) determining in the subject the presence or absence of an allele of the GHR gene, wherein the allele is correlated with a likelihood of having an increased or decreased positive response to an agent capable of ameliorating obesity or a symptom thereof; and
(b) selecting or determining an effective amount of said agent to administer to said subject.

18. A method for treating a subject suffering from diabetes, the method comprising:
(a) determining in the subject the presence or absence of an allele of the GHR gene, wherein the allele is correlated with a likelihood of having an increased or decreased positive response to an agent capable of ameliorating diabetes or a symptom thereof; and
(b) selecting or determining an effective amount of said agent to administer to said subject.

19. A method for treating a subject suffering from infection, the method comprising:
(a) determining in the subject the presence or absence of an allele of the GHR gene, wherein the allele is correlated with a likelihood of having an increased or decreased positive response to an agent capable of ameliorating infection or a symptom thereof; and
(b) selecting or determining an effective amount of said agent to administer to said subject.

20. A method for treating a subject suffering from an acromegaly or gigantism condition, the method comprising:
(a) determining in the subject the presence or absence of an allele of the GHR gene, wherein the allele is correlated with a likelihood of having an increased or decreased positive response to an agent capable of ameliorating an acromegaly or gigantism condition, or a lactogenic, diabetogenic, lipolytic and protein anabolic symptom associated therewith; and
(b) selecting or determining an effective amount of said agent to administer to said subject.

21. A method for treating a subject suffering from a condition associated with abnormal sodium or water retention, the method comprising:
(a) determining in the subject the presence or absence of an allele of the GHR gene, wherein the allele is correlated with a likelihood of having an increased or decreased positive response to an agent capable of ameliorating said condition associated with abnormal sodium or water retention; and
(b) selecting or determining an effective amount of said agent to administer to said subject.

22. A method for treating a subject suffering from a metabolic syndrome, the method comprising:
(a) determining in the subject the presence or absence of an allele of the GHR gene, wherein the allele is correlated with a likelihood of having an increased or decreased positive response to an agent capable of ameliorating said metabolic syndrome or a symptom thereof; and
(b) selecting or determining an effective amount of said agent to administer to said subject.

23. A method for treating a subject suffering from a mood or sleep disorder, the method comprising:
(a) determining in the subject the presence or absence of an allele of the GHR gene, wherein the allele is correlated with a likelihood of having an increased or decreased positive response to an agent capable of ameliorating said mood or sleep disorder or a symptom thereof; and
(b) selecting or determining an effective amount. of said agent to administer to said subject.

24. A method for treating a subject suffering from cancer, the method comprising:
(a) determining in the subject the presence or absence of an allele of the GHR gene, wherein the allele is correlated with a likelihood of having an increased or decreased positive response to an agent capable of treating a cancer; and
(b) selecting or determining an effective amount of said agent to administer to said subject.

25. A method for treating a subject suffering from a cardiac disease or hypertension, the method comprising:
(a) determining in the subject the presence or absence of an allele of the GHR gene, wherein the allele is correlated with a likelihood of having an increased or decreased positive response to an agent capable of ameliorating said cardiac disease or hypertension, or a symptom thereof; and
(b) selecting or determining an effective amount of said agent to administer to said subject.

26. The method of any one of claims 16 to 25, further comprising (c) administering said effective amount of said agent to said subject.

27. The method of any one of the above claims, wherein the method comprises determining whether the DNA of a subject encodes a GHR polypeptide having a deletion in exon 3.

28. The method of any one of the above claims, wherein the method comprises determining in the subject the presence or absence of the GHRd3 allele.

29. The method of claims 27 or 28, comprising determining whether a subject is a heterozygote or a homozygote for the GHRd3 allele.

30. The method of claims 27 to 29, wherein said determining step comprises detecting a GHRd3 nucleic acid in a sample.

31. The method of claim 30, wherein said determining step further comprises performing a hybridization assay.

32. The method of claims 27 to 29, wherein said determining comprises detecting a GHRd3 polypeptide in a sample.

33. The method of claim 32, wherein said determining step comprises detecting binding of an antibody to a GHRd3 polypeptide in a sample.

34. The method of any one of the above claims, wherein the subject is a human subject.

35. The method of claim 16, wherein the subject has ISS.

36. The method of claim 16, wherein the subject has a height less than about 2 standard deviations below normal for age and sex.

37. The method of claim 16 or 26, further comprising detecting whether the subject has a height less than about 2 standard deviations below normal for age and sex

38. The method of any one of the above claims, wherein said agent is a recombinant growth hormone polypeptide, or a fragment or variant thereof.

39. The method of claim 38, wherein the effective amount of GH is greater than about 0.2 mg/kg/week.

40. The method of claim 38, wherein, the effective amount of GH is greater than about 0.25 mg/kg/week.

41. The method of claim 38, wherein the effective amount of GH is greater than or equal to about 0.3 mg/kg/week.

42. The method of claim 38, wherein the effective amount of GH is between about 0.001 mg/kg/day and about 0.2 mg/kg/day.

43. The method of claim 38, wherein the effective amount of GH is between about 0.01 mg/kg/day and about 0.1 mg/kg/day.


SEQUENCE LISTING (6 sequences)

SEQ ID NO: 1 - DNA, 4414 bp, Homo sapiens
  CDS (44)..(1960)
  misc_feature (488)..(742): FNS; Region Fibronectin type 3 domain
ccgcgctctctgatcagaggcgaagctcggaggtcctacaggtatggatctctggcagctgctgttgaccttggcactggcaggatcaagtgatgctttttctggaagtgaggccacagcagctatccttagcagagcaccctggagtctgcaaagtgttaatccaggcctaaagacaaattcttctaaggagcctaaattcaccaagtgccgttcacctgagcgagagactttttcatgccactggacagatgaggttcatcatggtacaaagaacctaggacccatacagctgttctataccagaaggaacactcaagaatggactcaagaatggaaagaatgccctgattatgtttctgctggggaaaacagctgttactttaattcatcgtttacctccatctggataccttattgtatcaagctaactagcaatggtggtacagtggatgaaaagtgtttctctgttgatgaaatagtgcaaccagatccacccattgccctcaactggactttactgaacgtcagtttaactgggattcatgcagatatccaagtgagatgggaagcaccacgcaatgcagatattcagaaaggatggatggttctggagtatgaacttcaatacaaagaagtaaatgaaactaaatggaaaatgatggaccctatattgacaacatcagttccagtgtactcattgaaagtggataaggaatatgaagtgcgtgtgagatccaaacaacgaaactctggaaattatggcgagttcagtgaggtgctctatgtaacacttcctcagatgagccaatttacatgtgaagaagatttctactttccatggctcttaattattatctttggaatatttgggctaacagtgatgctatttgtattcttattttctaaacagcaaaggattaaaatgctgattctgcccccagttccagttccaaagattaaaggaatcgatccagatctcctcaaggaaggaaaattagaggaggtgaacacaatcttagccattcatgatagctataaacccgaattccacagtgatgactcttgggttgaatttattgagctagatattgatgagccagatgaaaagactgaggaatcagacacagacagacttctaagcagtgaccatgagaaatcacatagtaacctaggggtgaaggatggcgactctggacgtaccagctgttgtgaacctgacattctggagactgatttcaatgccaatgacatacatgagggtacctcagaggttgctcagccacagaggttaaaaggggaagcagatctcttatgccttgaccagaagaatcaaaataactcaccttatcatgatgcttgccctgctactcagcagcccagtgttatccaagcagagaaaaacaaaccacaaccacttcctactgaaggagctgagtcaactcaccaagctgcccatattcagctaagcaatccaagttcactgtcaaacatcgacttttatgcccaggtgagcgacattacaccagcaggtagtgtggtcctttccccgggccaaaagaataaggcagggatgtcccaatgtgacatgcacccggaaatggtctcactctgccaagaaaacttccttatggacaatgcctacttctgtgaggcagatgccaaaaagtgcatccctgtggctcctcacatcaaggttgaatcacacatacagccaagcttaaaccaagaggacatttacatcaccacagaaagccttaccactgctgctgggaggcctgggacaggagaacatgttccaggttctgagatgcctgtcccagactatacctccattcatatagtacagtccccacagggcctcatactcaatgcgactgccttgcccttgcctgacaaagagtttctctcatcatgtggctatgtgagcacagaccaactgaacaaaatcatgccttagcctttctttggtttcccaagagctacgtatttaatagcaaagaattgactggggcaataacgtttaagccaaaacaatgtttaaaccttttttgggggagtgacaggatggggtatggattctaaaatgccttttcccaaaatgttgaaatatgatgttaaaaaaataagaagaatgcttaatcagatagatattcctattgtgcaatgtaaatattttaaagaattgtgtcagactgtttagtagcagtgattgtcttaatattgtgggtgttaatttttgatactaagcattgaatggctatgtttttaatgtatagtaaatcacgctttttgaaaaagcgaaaaaatcaggtggcttttgcggttcaggaaaattgaatgcaaaccatagcacaggctaattttttgttgtttcttaaataagaaacttttttatttaaaaaactaaaaactagaggtgagaaatttaaactataagcaagaaggcaaaaatagtttggatatgtaaaacatttactttgacataaagttgataaagattttttaataatttagacttcaagcatggctattttatattacactacacactgtgtactgcagttggtatgacccctctaaggagtgtagcaactacagtctaaagctggtttaatgttttggccaatgcacctaaagaaaaacaaactcgttttttacaaagcccttttatacctccccagactccttcaacaattctaaaatgattgtagtaatctgcattattggaatataattgttttatctgaatttttaaacaagtatttgttaatttagaaaactttaaagcgtttgcacagatcaacttaccaggcaccaaaagaagtaaaagcaaaaaagaaaacctttcttcaccaaatcttggttgatgccaaaaaaaaatacatgctaagagaagtagaaatcatagctggttcacactgaccaagatacttaagtgctgcaattgcacgcggagtgagttttttagtgcgtgcagatggtgagagataagatctatagcctctgcagcggaatctgttcacacccaacttggttttgctacataattatccaggaagggaataaggtacaagaagcattttgtaagttgaagcaaatcgaatgaaattaactgggtaatgaaacaaagagttcaagaaataagtttttgtttcacagcctataaccagacacatactcatttttcatgataatgaacagaacatagacagaagaaacaaggttttcagtccccacagataactgaaaattatttaaaccgctaaaagaaactttctttctcactaaatcttttataggatttatttaaaatagcaaaagaagaagtttcatcattttttacttcctctctgagtggactggcctcaaagcaagcattcagaagaaaaagaagcaacctcagtaatttagaaatcattttgcaatcccttaatatcctaaacatcattcatttttgttgttgttgttgttgttgagacagagtctcgctctgtcgccaggctagagtgcggtggcgcgatcttgactcactgcaatctccacctcccacaggttcaggcgattcccgtgcctcagcctcctgagtagctgggactacaggcacgcaccaccatgccaggctaatttttttgtattttagcagagacggggtttcaccatgttggccaggatggtctcgagtctcctgacctcgtgatccacccgactcggcctcccaaagtgctgggattacaggtgtaagccaccgtgcccagccctaaacatcattcttgagagcattgggatatctcctgaaaaggtttatgaaaaagaagaatctcatctcagtgaagaatacttctcattttttaaaaaagcttaaaactttgaagttagctttaacttaaatagtatttcccatttatcgcagaccttttttaggaagcaagcttaatggctgataattttaaattctctctcttgcaggaaggactatgaaaagctagaattgagtgtttaaagttcaacatgttatttgtaatagatgtttgatagattttctgctactttgctgctatggttttctccaagagctacataatttagtttcatataaagtatcatcagtgtagaacctaattcaattcaaagctgtgtgtttggaagactatcttactatttcacaacagcctgacaacatttctatagccaaaaatagctaaatacctcaatcagtctcagaatgtcattttggtactttggtggccacataagccattattcactagtatgactagttgtgtctggcagtttatatttaactctctttatgtctgtggattttttccttcaaagtttaataaatttattttcttggattcctgataatgtgcttctgttatcaaacaccaacataaaaatgatctaaacc

SEQ ID NO: 2 - protein, 638 aa, Homo sapiens
MDLWQLLLTLALAGSSDAFSGSEATAAILSRAPWSLQSVNPGLKTNSSKEPKFTKCRSPERETFSCHWTDEVHHGTKNLGPIQLFYTRRNTQEWTQEWKECPDYVSAGENSCYFNSSFTSIWIPYCIKLTSNGGTVDEKCFSVDEIVQPDPPIALNWTLLNVSLTGIHADIQVRWEAPRNADIQKGWMVLEYELQYKEVNETKWKMMDPILTTSVPVYSLKVDKEYEVRVRSKQRNSGNYGEFSEVLYVTLPQMSQFTCEEDFYFPWLLIIIFGIFGLTVMLFVFLFSKQQRIKMLILPPVPVPKIKGIDPDLLKEGKLEEVNTILAIHDSYKPEFHSDDSWVEFIELDIDEPDEKTEESDTDRLLSSDHEKSHSNLGVKDGDSGRTSCCEPDILETDFNANDIHEGTSEVAQPQRLKGEADLLCLDQKNQNNSPYHDACPATQQPSVIQAEKNKPQPLPTEGAESTHQAAHIQLSNPSSLSNIDFYAQVSDITPAGSVVLSPGQKNKAGMSQCDMHPEMVSLCQENFLMDNAYFCEADAKKCIPVAPHIKVESHIQPSLNQEDIYITTESLTTAAGRPGTGEHVPGSEMPVPDYTSIHIVQSPQGLILNATALPLPDKEFLSSCGYVSTDQLNKIMP

SEQ ID NO: 3 - protein, 638 aa, Homo Sapiens; identical to SEQ ID NO: 2
  Product (1)..(638): Growth Hormone Receptor

SEQ ID NO: 4 - DNA, 6789 bp, Homo Sapiens
  gene (1)..(6789): Growth Hormone Receptor (GHR)
aacttanagtatcaaagcagcaagtagatttgaaggaattgttacaatgcaattttgctttcccgccactttaaaatcaaggtgtagtactttatttactttaggaaaatgtttgctttttgtcataattccttattgcatatgagagtaaatgatctatagatgaagataataataaaatttagagagagaataaaaaagaaacactttcacagctgaaaggctgcttcccagttagctaactgggaggagttactgaaaaagtacattgaaaagcggctcaggggcaggtgaattggactcaccaggctctgacattcagagagatgggaatgagtcagctcactgtccagcacatctttattttatttctctttcttgttttatatcagaaatagatttcttggcattgttactgtgggtttctattaaggactgaacaaaagtattaataatctgagagtatgtaaaaaaaaattcattttctcctactatactctcataacacagaatattttggtgaccagagatcaccaaaatgtgtgtggtgtcaacgaaaagagtcaaactctctaaaatatttgaagagattttttctgagccaaatgtgagtgaacatggcctgtgacatagccctcaggaggtcctgagaacatgtgcccaaggtggtcagggtacagcttggtttttatatattttagggaggcataagacatcaatcaaatacatttaagaaatacgttgatttggttcagaaaggcaggacaactcaaatggggagcttccaggctataggtaaatttaaacattttctggttgacaattagttgagtttgtctgaagacctgggattaatggaaaggactattcaggttaagatatgtttcttattggacctaaaactgtgcctggctcttagttgattactgcctggatctgggaaggaaggaaggaaaacaaagggggaaggggattctctatagaatgtggatttttcccataagagactttgtagggcaatttcaaggcatggcaaggaaatatactttggggctaatattttnccttgtctcataatgttatgccagagtcatattgaaaagcaagtcacaatatacaaggtcaaataaaaccatctgatgagaacccatggtttgtagggcatgactccccagaacccttaggtaggaatttgggcaagataaaaaatcggaacttagtcctcggcgggaatctctccccacacaaattctccaacagattcttcagtgggacaccaactgggtggttctcaaattcaattcaattctgaccaatctacctatctacctggaaatagcatcagataaccacaggtttacggctcattccaacaatactgtcccccacttcagatgccaactgcaagtaataggttgttacctatacttctagccagtcagctgtnaantggtgttcccacaacctccccctccggtttgataatttgagacagcttgcttacatgtaccagcttattagaaaggatattacaaaggacacagatgaagagatggatagggtaaggtatgtgggttggagttgcagagtttccatgacctctctgagtgcagcatcttcatgtgttcagctatccagaatctctcggattaagacattggccactggtgatcaaattaaccttgagtccctctccccttcctgaggttggagagtggggctgaagtgtctcaacctctaatcaactcttggtctttcctgtgaccatgccccatcctgaggctctccaggagcccccaggcatcagtcaactcattagcatacgaaagacacttatcactacagagattcgaaggattttaggaactgtgtcaagaaacggagacaaggtcaaatatgtatttcacaatatcaccagtagtttcactgggaggtaaaactcagtgtttactgtgggcctgagccatgctgaccctctaagaataacttagaggtaacgtgatcagatgtggggaattctggagaaacacctttcaccaccaagcccagacaagagatgcatacttttctagctgggatgcttacaaagcaacccactctaatacttcaaggtagagtgacactacattcatcatttttcattttttcctgttttttatgccatctactactaatgtcaatcaaattacgactgtgtttatagtggatgaattatggaccatctcacaccataaagttctgtttctctcatgttgagcttttcacctcccttcattccctccctacttccaggatcattcacatgtttatttctaaaaataaactttttttactgaactttttttcatactgtttaaaaagaatttatatttctcttcattcttacagataagattcaagtttaaactcaaataatgtaggaaatctttttttaaaaaattgttccctactgtgtctaggcgtgagacccaaaagtaattaagaccaggttttcatttgctgtgatttgtgtgagttctttttagaggttaggtgcaattttaatttttaaaagggggattattatgagaggagaaatcatactttatcatttgaaaatgatgccataacaggtgttagcagaaaaatcaaactgtaaaatattttaaagagatttattctgagccaatataagtgactgtggccccattgaaatgagcgagttccctgatccctctcacagagcttgcgacagggatgtggctcacctgttcagttgccccaccgctcaaacccctagggggagaatacagacggtcaggtgcaaaggctggggcaagtgccttggccccttggccccttagccccgaggtagtgtctaggggtggggtgcctgcaaccccagtgttacaaagttctttcagctttgcagtccacggacagcttgagtgttaatcagctcaatggaccctctgccttatagcaaaggcagagggccagtgtgacagctttctgtatcccaagctcttgcccagtgtcctagaaaaaacagatcatacaggggctcgaaggatgagtgcaaggttttattgagtagtggaggtggctctcagcaagatggatggggagtgggaagtggggatggagtgggaaggtgaacttcctctgaagtcgggcagcccagtggctggactcttctccaacctcccccaggcaagctcctctcagcgtccagatgttcctcttccctctctctctctgccgcatcatttcaccatctgtctgctggtcagctggcttgctggtgtgctggtctgttggtctgctggtctgcttctggaacctcaggttcagagtttatatgagtgcacgatagggggtgttttgggccaaaaggtagctttttggacatgaaaacggaaatgcctgttcccatttagggctgcaggtcttcaggcttgagggtggggcctttgcccaggaactaccctcttctacccagtgtttccctgtctcctgtccatatcaccagtattcacagtctcaaggagtcttgagaaagtgtgcccaaggccgtcagattcagtttggttctgtatgtttcagggaggcaggaattacaggcaaagacataaatcagtacatggaaggtatacattggttcactctgaaaaggcaggatgtcttgaagtggggacttgcaggtcatagtttggttcagagattctttaatctgcagttggttaaaggaacaaaactgtacagaagcttcgagttagcaaaaagaaatatttaaattaagataaggatgctatgtcagagtcagccacaaaatgacctgtttagcaagattaatggcctataggtgtgacttaacccttgccttgcatggcctaaggtcttgtttataatttagtatcttattgcccaaagagtctatttagtcagtcttatgatctctactttaacattaatgctggtcacttgtgcctaaactccaaaggggaggtatatccaacctgccttcccattgtggccaggaacctttctctggagtccccttggccaagaaggggtccattcggttggtttgggaagctgaggattttgtttttagtttacacagggtcatatcagattgttttgatggggatgactaatggttttcttctctttctgtttcagccacagcagctatccttagcagagcaccctggagtctgcaaagtgttaatccaggcctaaagacaagtaagaatttcagtcctttttcttccttcaatgatattttccatgttttagtgtaattaagctactatcctttctctattttatttgggatggtagtaactggaatagtgactgagttgaaattttataggcaagcaaaacattttttaaggatttattttttaacttctgatatagtttggatgtttgtcccttccaaatctcatgtaatccccaatgttggaagtggagactgggaggagatgtttgggtcatgtgggcagattcctcatgaatggtttagcaccctcctctttgtgctgtcctcaccatgagtgagttctcatgagatctggttgtttaaaagtgtgtggcacctcccccttcaatctcttgctcccactctcgccctgtgagacacctgctccgcttcaccatgattataggcttcctgaggctttcaccagaagcagatgctaatacagcctgcagaactgtgagccatttaaatcatttttctttataaatcacccagcctcaggtacttttttatagcaatgaaagcaaactaatacaacttctgtgcaaggctgcttttttttctattttttgcttgtgcttgaaggttaagtaaggccaaattaatgaaggaggaaaaaagaggaaatgatacatcatggatcaacaattatttattgaatttaggaaactgcctctttttataaattctttttaaaattattttcattattatcttgaagtatttatctaaggtttacactggtagaaagttaaacttgtctctccaaccaaattgccttaagcttcaaaattatgccttattgtaagctctttcttaaccttaaaatgactttacacattccccgctggtcctttgacaatctcctcttcaaccacaagacagaaccccaccatcaactctgtggggaagcgtctccaaattctctagtcctgaacaacatgctgccttctctgcttccatggaactttgtcctttacaacatgatagcgtttgcctcctgacattttagtgtgtgtgttagccctgcatatagaactcaccagattgtgtggtctgcatgaatgaattaattctattgaactttaaggcaaagcctaaactttatgcttcttctaaatcccttacatctcctaaaaaaattctgatccatagtagtaggtacttgtttaattaaattttagggatggatatttttcatcagtggaagtatatgctagagtccatattatgcaataagggaagggaagacagtgtacctaaatcagttaagatattgctattcttgttgttattctaaatcagttaagatattgctattcttgttgttattctagagtcacgaaatcataatttgaattttatgactaaattgcagaattaatttccaatgtgagattttaacattatttccttggaggtgaccaaaaaggagagctggtactgtttttaacaactgtcattcaattgtcagttgtgccagaccacaaatcctttatagccctcctgtttaagaagcatctgacatgttaagctgctccctaattaacacagaggttgtaaaagaagtggctgtttggttctgtttgggtttcccagccagtatattccaaagccttttttcactcaacagatgagttatgtgctttatattctgtaaggaaatgagaagtaatcagttgaaaatgtgttactaatggtacatgcttcacattgaaaccatcctcctgacacaaacataatactttgcccttcactgtcccccaaagtggcagtaggatttctctaagtaattttctttacttatatgagtgcaggatagggggtgttttgggccaaaaggtagctttttggacatgaaaacggaaatgcctgttcccatttagggctgcaggtcttcaggcttgagggtggggcctttgcccaggaactaccctcttctacccagtgtttccctgtctcctgtccatatcaccagtattcacagtctcaaggagtcttgagaaagtgtgcccaaggccgtcagattcagtttggttctgtatgtcacagggtctaagaagcgtaaacattgtgccttgttgaaatacagcctctaggtatggaggatgtgttgaacaacttcctaccagtcatttggcatatgttgatttcctgtcttcatgatacgtaagacgactagctaattatcattcatatgtggtaagtcacatagatactgacttcccctatctttccagctttttcttatcaaaagtcacctgctctctgtcccaggaacgactggctaaagtaacctatatcagtgtctgtaacagtgggcacctatcatagtgcacatgcttgaacatatcattgccttttatcatcacgagcctcacatccagatgtgacagactcaagtgctcacatcacctcactctgtcactgtatacattgttaccgtgtcacaaatatttaacagtctgctgtgtactcagtctttagctgtgtgccctgagggagacagagtaagatactgccttgacatcaaggagctc

SEQ ID NO: 5 - protein, 19 aa, Homo Sapiens
PQQLSLAEHPGVCKVLIQA

SEQ ID NO: 6 - DNA, 1474 bp, Homo sapiens
  GENE (1)..(1474): Growth Hormone Receptor (GHR) contains a deletion of exon 3
aatctttttttaaaaaattgttccctactgtgtctaggcgtgagacccaaaagtaattaagaccaggttttcatttgctgtgatttgtgtgagttctttttagaggttaggtgcaattttaatttttaaaagggggattattatgagaggagaaatcatactttatcatttgaaaatgatgccataacaggtgttagcagaaaaatcaaactgtaaaatattttaaagagatttattctgagccaatataagtgactgtggccccattgaaatgagcgagttccctgatccctctcacagagcttgcgacagggatgtggctcacctgttcagttgccccaccgctcaaacccctagggggagaatacagacggtcaggtgcaaaggctggggcaagtgccttggccccttggccccttagccccgaggtagtgtctaggggtggggtgcctgcaaccccagtgttacaaagttctttcagctttgcagtccacggacagcttgagtgttaatcagctcaatggaccctctgccttatagcaaaggcagagggccagtgtgacagctttctgtatcccaagctcttgcccagtgtcctagaaaaaacagatcatacaggggctcgaaggatgagtgcaaggttttattgagtagtggaggtggctctcagcaagatggatggggagtgggaagtggggatggagtgggaaggtgaacttcctctgaagtcgggcagcccagtggctggactcttctccaacctcccccaggcaagctcctctcagcgtccagatgttcctcttccctctctctctctgccgcatcatttcaccatctgtctgctggtcagctggcttgctggtgtgctggtctgttggtctgctggtctgcttctggaacctcaggttcagagtttatatgagtgcaggatagggggtgttttgggccaaaaggtagctttttggacatgaaaacggaaatgcctgttcccatttagggctgcaggtcttcaggcttgagggtggggcctttgcccaggaactaccctcttctacccagtgtttccctgtctcctgtccatatcaccagtattcacagtctcaaggagtcttgagaaagtgtgcccaaggccgtcagattcagtttggttctgtatgtcacagggtctaagaagcgtaaacattgtgccttgttgaaatacagcctctaggtatggaggatgtgttgaacaacttcctaccagtcatttggcatatgttgatttcctgtcttcatgatacgtaagacgactagctaattatcattcatatgtggtaagtcacatagatactgacttcccctatctttccagctttttcttatcaaaagtcacctgctctctgtcccaggaacgactggctaaagtaacctatatcagtgtctgtaacagtgggcacctatcatagtgcacatgcttgaacatatcattgccttt